SEQUENCE LISTING (80 sequences)

SEQ ID NO: 1 - DNA, 7713 bp, Artificial
  Plasmid TRCBA-int-luc mut (654 C-T)
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggatattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcgctattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatactttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgcggataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgct

SEQ ID NO: 2 - DNA, 7713 bp, Artificial
  Plasmid TRCBA-int-luc (wt)
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggatattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcgctattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatactttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgcggataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgct

SEQ ID NO: 3 - DNA, 7713 bp, Artificial
  Plasmid TRCBA-int-luc (654 C-T, 657 TA-GT)
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggatattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcgctattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatactttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgcggataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgct

SEQ ID NO: 4 - DNA, 5860 bp, Artificial
  Plasmid GL3-int-Luc mut (654 C-T)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 5 - DNA, 5860 bp, Artificial
  Plasmid GL3-int-Luc (wt)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 6 - DNA, 5860 bp, Artificial
  Plasmid GL3-int-Luc (654 C-T, 657 TA-GT)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 7 - DNA, 6683 bp, Artificial
  Plasmid GL3-2int-fron-sph (mut)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 8 - DNA, 7547 bp, Artificial
  Plasmid GL3-3int-2fron-sph (mut)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatgagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatgcatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 9 - DNA, 5860 bp, Artificial
  Plasmid GL3-int-luc A (mut)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 10 - DNA, 5860 bp, Artificial
  Plasmid GL3-int-Luc B
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 11 - DNA, 5860 bp, Artificial
  Plasmid GL3-int-Luc C
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 12 - DNA, 5833 bp, Artificial
  Plasmid GL3-int-fron (mut)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 13 - DNA, 6710 bp, Artificial
  Plasmid GL3-2int-sph (mut)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 14 - DNA, 6710 bp, Artificial
  Plasmid GL3-2int-Sph-C
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 15 - DNA, 5660 bp, Artificial
  Plasmid GL3-sint200-sph (mut)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 16 - DNA, 5660 bp, Artificial
  Plasmid GL3-sint200-sph (657 GT)
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 17 - DNA, 5436 bp, Artificial
  Plasmid GL3-sint425-sph
ggtaccgagctcttacgcgtgctagcccgggctcgagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttcctgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 18 - DNA, 850 bp, Artificial
  Mutant beta globin intron (654C-T)
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 19 - DNA, 850 bp, Homo sapiens
  misc_feature (1)..(850): Wild-type beta-globin intron
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 20 - DNA, 850 bp, Artificial
  Double mutant beta globin intron (654C-T 657TA-GT)
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 21 - DNA, 2503 bp, Artificial
  Luciferase with mutant beta-globin intron (654C-T)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 22 - DNA, 2503 bp, Artificial
  Luciferase with wild-type beta-globin intron
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 23 - DNA, 2503 bp, Artificial
  Luciferase with double mutant beta-globin intron (654C-T 657TA-GT)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 24 - DNA, 3355 bp, Artificial
  Luciferase with mutant beta-globin intron (654C-T) with mutant beta-globin intron (654C-T) upstream to translation start
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 25 - DNA, 4219 bp, Artificial
  Luciferaase with mutant beta-globin intron (654C-T) and two mutant beta-globin introns (654C-T) upstream to translation start
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatgagcttgtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatgcatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 26 - DNA, 2503 bp, Artificial
  Luciferase with mutant beta-globin intron (654C-T) alternative location A
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 27 - DNA, 2503 bp, Artificial
  Luciferase with mutant beta-globin intron (654C-T) alternative location B
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 28 - DNA, 2503 bp, Artificial
  Luciferase with mutant beta-globin intron (654C-T) alternative location C
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 29 - DNA, 2505 bp, Artificial
  Luciferase with mutant beta-globin intron upstream of translation start
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 30 - DNA, 3353 bp, Artificial
  Luciferase with two mutant beta-globin introns (654C-T)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 31 - DNA, 3353 bp, Artificial
  Luciferase with two mutant beta-globin introns (654C-T)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 32 - DNA, 2303 bp, Artificial
  Luciferase with mutant beta-globin intron (654C-T and 200 basepair deletion)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 33 - DNA, 2303 bp, Artificial
  Luciferase with double mutant beta-globin intron (654C-T 657TA-GT and 200 basepair deletion)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagtgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 34 - DNA, 2079 bp, Artificial
  Luciferase with mutant beta-globin intron (654C-T and 425 basepair deletion)
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccaggtgagtctatgggacccttgatgttttctttcctgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 35 - DNA, 7449 bp, Artificial
  Plasmid TRCBA with antitrypsin and mutant beta-globin intron (654C-T)
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttcaatattggccattagccatattattcattggttatatagcataaatcaatattggatattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtatgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttactttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttggggattttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagagccatgaaggctgccaggaactcctccgtaccctcaaccagccagacagccagctccagctgaccaccggcaatggcctgtgcctcagcgagggcctgaagcaagtggataagtttttggaggatgttaaaaagttgtaccactcataagccttcactgtcaacttcggggacaccgaagaggccaagaaacagatcaacgattacgttgagaagggtactcaagggaaaatggtggatgtggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttagtcatgtttaacatccagcactgtaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagaagctttccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccgtggtcacagaggaggcacccctgaagctctccaatgccgtgcataaggctgtgctgaccatcgacgagaaagggactgaagctgctggggccatgtttttagaggccatacccatgtctatcccccccgaggtcaaggtcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtaaccccccccccccctgcaggggccctcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagcctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcgctattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatactttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgcggataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgct

SEQ ID NO: 36 - DNA, 2107 bp, Artificial
  Antitrypsin with mutant beta-globin intron (654C-T)
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagagccatgaaggctgccaggaactcctccgtaccctcaaccagccagacagccagctccagctgaccaccggcaatggcctgtgcctcagcgagggcctgaagcaagtggataagtttttggaggatgttaaaaagttgtaccactcataagccttcactgtcaacttcggggacaccgaagaggccaagaaacagatcaacgattacgttgagaagggtactcaagggaaaatggtggatgtggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttagtcatgtttaacatccagcactgtaaggtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagaagctttccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccgtggtcacagaggaggcacccctgaagctctccaatgccgtgcataaggctgtgctgaccatcgacgagaaagggactgaagctgctggggccatgtttttagaggccatacccatgtctatcccccccgaggtcaaggtcaacaaaccctttgtcttcttaatgattgaacaaaataccaagtctcccctcttcatgggaaaagtggtgaatcccacccaaaaataa

SEQ ID NO: 37 - DNA, 18 bp, Artificial
  Synthetic oligonucleotide
gctattaccttaacccag

SEQ ID NO: 38 - DNA, 18 bp, Artificial
  Synthetic oligonucleotide
gcacttaccttaacccag

SEQ ID NO: 39 - DNA, 18 bp, Artificial
  Synthetic oligonucleotide
caagggtcccatagtctc

SEQ ID NO: 40 - DNA, 18 bp, Artificial
  Synthetic oligonucleotide
gaaagagatgagggaaag

SEQ ID NO: 41 - DNA, 18 bp, Artificial
  Synthetic oligonucleotide
gaaagagaagagggaaag

SEQ ID NO: 42 - DNA, 18 bp, Artificial
  Synthetic oligonucleotide
cctcttacctcagttaca

SEQ ID NO: 43 - DNA, 18 bp, Artificial
  Synthetic oligonucleotide
ctgtgggagtaagataag

SEQ ID NO: 44 - DNA, 18 bp, Artificial
  Synthetic oligonucleotide
gctcttaccttaacccag

SEQ ID NO: 45 - DNA, 18 bp, Artificial
  Synthetic oligonucleotide
gcaattaccttaacccag

SEQ ID NO: 46 - DNA, 18 bp, Artificial
  Synthetic oligonucleotide
caagggtcccatagactc

SEQ ID NO: 47 - DNA, 18 bp, Artificial
  Synthetic oligonucleotide
gaaagagattagggaaag

SEQ ID NO: 48 - DNA, 18 bp, Artificial
  Synthetic oligonucleotide
ctgtgggaggaagataag

SEQ ID NO: 49 - DNA, 18 bp, Artificial
  Synthetic oligonucleotide
cctcttacatcagttaca

SEQ ID NO: 50 - DNA, 850 bp, Artificial
  IVS2-654 intron with 564CT mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 51 - DNA, 850 bp, Artificial
  IVS2-654 intron with 657G mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaagagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 52 - DNA, 850 bp, Artificial
  IVS2-654 intron with 658T mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaattgcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 53 - DNA, 650 bp, Artificial
  IVS2-654 intron with 200 bp deletion
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 54 - DNA, 426 bp, Artificial
  IVS2-654 intron with 425 bp deletion
gtgagtctatgggacccttgatgttttctttcctgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 55 - DNA, 850 bp, Artificial
  IVS2-654 intron with 6A mutation
gtgagactatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 56 - DNA, 850 bp, Artificial
  IVS2-654 intron with 564C mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 57 - DNA, 850 bp, Artificial
  IVS2-654 intron with 841A mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttactcccacag

SEQ ID NO: 58 - DNA, 850 bp, Artificial
  Mutant beta globin intron (705T-G)
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 59 - DNA, 850 bp, Artificial
  IVS2-705 intron with 564 CT mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcttctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 60 - DNA, 850 bp, Artificial
  IVS2-705 intron with 657G mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaagagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 61 - DNA, 850 bp, Artificial
  IVS2-705 intron with 658T mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaattgcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 62 - DNA, 850 bp, Artificial
  IVS2-705 intron with 657GT mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaagtgcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 63 - DNA, 650 bp, Artificial
  IVS2-705 intron with 200 bp deletion
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagttgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 64 - DNA, 426 bp, Artificial
  IVS2-705 intron with 425 bp deletion
gtgagtctatgggacccttgatgttttctttcctgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 65 - DNA, 850 bp, Artificial
  IVS2-705 intron with 6A mutation
gtgagactatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 66 - DNA, 850 bp, Artificial
  IVS2-705 intron with 564C mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctcatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 67 - DNA, 850 bp, Artificial
  IVS2-705 intron with 841A mutation
gtgagtctatgggacccttgatgttttctttccccttcttttctatggttaagttcatgtcataggaaggggagaagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgaggtaagaggtttcatattgctaatagcagctacaatccagctaccattctgcttttattttatggttgggataaggctggattattctgagtccaagctaggcccttttgctaatcatgttcatacctcttatcttactcccacag

SEQ ID NO: 68 - DNA, 196 bp, Artificial
  IVS2-654 intron 197 bp
gtgagtctatgggacccttgatgttcttttaatatacttttttgtttatcttatttctaatactttccctcttctctttctttcaggtgattgactgactgggttaaggtaatagcgccgttgaaaacctcagccgtatagtccaagctaggcccttttgctaatcatgttcatacctcttatcttcctcccacag

SEQ ID NO: 69 - DNA, 247 bp, Artificial
  IVS-654 intron 247 bp
gtgagtctatgggacccttgatgttcttttaatatacttttttgtttatcttatttctaatactttccctaatctctttctttcagggcaataatgatacaatgtatcatgcctctttgcaccattctaaagaataacagtgataatttctgggttaaggtaatagcaatatttctgcatataaatatttagtccaagctaggcccttttgctaatcatgttcatacctcttatcttcctcccacag

SEQ ID NO: 70 - DNA, 14667 bp, Homo sapiens
  misc_feature (1)..(14667): CFTR gene exon 19
gtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatataatgggtttattttaaatgtgattgtacttgcagaatatctaattaattgctaggttaataactaaagaagccattaaataaatcaaaattgtaacatgttttagatttcccatcttgaaaatgtcttccaaaaatatcttattgctgactccatctattgtcttaaattttatctaagttccattctgccaaacaagtgatactttttttctagcttttttcagtttgtttgttttgtttttctttgaagttttaattcagacatagattattttttcccagttatttactatatttattaagcatgagtaattgacattattttgaaatccttcttatggatcccagcactgggctgaacacatagaaggaacttaatatatactgatttctggaattgattcttggagacagggatggtcattatccatatacttcaggctccataaacatatttcttaattgccttcaaatccctattctggactgctctataaatctagacaagagtattatatattttgattgatattttttagataaaataaaagggagctgaaaactgaattgcaaactgaattttaaaactttatctctctgtggttaattgcaaacacagatacaaaaatatagagagagatacagttagtaaagatgttaggtcaccgttactaacactgacatagaaacagttttgctcatgagtttcagaatatatgagtttgattttgcccatggattttagaatatttgataaacatttaatgcattgtacaaattctgtgaaaacatatatataggatgtgcgaaaagtccctgtgtatcatgtgaaatggcttaaaacagaacaccataggtattcatatcagtgaataccataggtagctgaaagtgttttttcctggggtcgccaagatgaatgccaaaagtgatatcattattataaacaatagccagaataggttggtataaacctggtagaaagccttgataaattgactttctctcctcctgacatcctgccacccctttgctttgctgatgctcatttgtccactaaattaaactcaagcaagccctagtaaagtaatagaatttgtggagtcctcattagtataggaagtttccctgatgtgagattagtaattagagatgtagcaaaatgagaaagaagtaatatgcttagatatttcattttctctgaacctgtatatacaaaataggccatgcgtgttcagtaactattcactgcaaggcactctctaggtactttgggggaattggaaattactcacataaggctatggattgtgccatttgtcaaaagacaaaatgacaacaaatttagtttaaagacctcagtcagctttattttctattctagatttggacagtccttcatttcacaaattggagtaagtgttccaataagttgagcaaaggagcttggctttatagacccaaaaaaagggccaaaggaagcagaaacaaagaacaataagagaattggtcatttcaaagttacttttcttgaaaggtggggacaaggagacagaataatagaaaagtcactgattggttaacattggattaagaattaaaacagaggaaactttaagattgaagtttgaaactgacttgtttgggaaatcaggctgtcttctttcttgatttcttagaaggccggataacaactgagttttgctttggtgaacatgggtgactccatttttacttttagtctggtctgttgaggcctcgtgagagagcttaatctaaaacaatgacttcctataatttttgtttgacacatccaaagagggactctaatatttattgagagcttatcatatcttaagtactgtttaaacacttttatttgctattacatttgatcttattataactctaaaggcagaaatgattgcttttattttccacaatggaggaaactgaggttcaattaagtgagtaaggaagcagggatcttaaacccagataccattgctcctctttaaaggtggaagaacagaaaacatggggcaggggaagagagaaagtttctgtcccaggacatgataatctaaaagggaaaacgtaagatccactgaaacctgaggcagatttattgtggcaataacaaagcttaagtttcacagaccttcatttgcctgagccaactttgaaggccatgtatctaattttgtttttataattctataatctttattcttgaaaagagccctccctccaaatttacaagctttgggcccccaaaatccttgaaatgcccttgaataagagatatccaggtaaatgctatgggaattcagaggaggaagcagttagtatcagttggcggagagttaggctattaagagaaggttttatataggaagtggcatttagaatgaagctttgagaactgagctgtgtatttgaacaagtaaaggtggtgttgcagaattttgctccttagttctattaaaaacccgggttcttgtcacatgatccggaaaatttaggcacacagatacattgaagcatgagtagagcaggattttattgggcaaaaaggaaaaaaagaaaactcagcaaatcgagatggagtcttgctcacagattgaatcccaggccaccacaaaggaactgaagagatcgggcttctcccctgcataaggtgcaaattccccatggctccacccacttccccttagtgtgcatgtggggctccagtccacggtgggcatgcccagacaagccttgggcaggttccctcatctgtgcaaaagcatctgatgtaaacacttgaggggtggttcggagattctctgggacccttttattttcttatctgcctaggcatttggctgtctcagtgggtgggaaagggtgctccaggcaaagggcataacatgaggcaaagggcatgcacagaaaacagtgactggttcagtcaggttgggggatgccaaaggaagtaatgggagacaagattggagcaagatagataagagattgtggattttttttcttttttatctatataaatacagagacagggtctcactatgttgcccaggctggtctcaaactcctggcctcaagtgatcctcccacctcatcctcccaaagtgctaggattacaggcatgaggcactgtgcccaacctccaattttggattttgagagctaaagcaatatagtcgaaaactcagataatccaggtagattttgctattaggtgctatttggttcctggtacagagctaaaacccttggaatttcctaagtgataagagctacaggagcatcttttgttatatgtttccccccctagttcctgaaatagctctagagaaatacaggtgaataacatcctttgttattcatatcaagcccctatcaaccataccccagtttctatttatgaagtggcttttgggaagtccctaaagacaggagtggggaaaggctggttgtcagggggatgggttgaaactttcatcttccccccttgacctccagggagggatgagtggctgaaaattgtgtaaaatcaacaatggccagtgatttaatcaaccatgcctatgtaatgaagccacccgataagccttaactggaactttttggagagcctccaggctggtgaagacattgaggtgctcagaaggtggtattccagagagagcacagaatctctgttccccttcccacattcattttgctatgcatctctcccatctggctgttcttgagaggtatccgtttataataaactggtaacctagtaagtaaactgttaccctgagttctgtgagccattctagcaaattatcaaacctaaagagttcatggatacgtgcaatttacagatgcacagtcagaagcacagatgacaatctgggcttgccattggcatttgaagtgtgttgggaggcagtcttacaggaatgagcccttatcctgtggggtctatgctaataacagacagttgtcagcattgcttggtgtcgaaaacccacattgttggtgtcagaagtattgtcagtaggatagggaaaacagtttgttttctttttttagtggtctttggtcatctttaagagcagggcttctcaaagtgtggtccttgaaccagcatcacctgtaccacgtaagaacttatgagaaatgttcattcttgggccccaacaaagaattaaaaattctgagggtgtgaacggggtctgagtttcagcacaacttcccgaccatgctgatgcattcttgcccaagcatgaaagccctcccttgtttaagaaggccattagggccgggtgtggtggctcatgcttgtaatcgagcactttgagaggacatagtgggaggatcacttgagccctggagttctagacaagcctgggcaacatggcaaaatgctgtctccacaaaaatcacaaaaattaggtgggcgtgtgttgtgtgcctataggcccagctacttaggagactgaggcaggaggatcgcttgagcccaggagattaaggctgcagcgagctgtgatggcaccactacagcctggatgacagagtgagacactgtctcaaaaaaaaaaaagaaaaagaaaaagaaaaaagaaaggaaaatgaaaaagaacgccattaggtataaaggagcaatggtaaaagaccagttgcaaaaggttagggaatgggtggttactgaaataagaagctatgtagaacactagtgttggtggcaggaagtagaaagcaagagcactgctctgtgggggatggtcatagcaaatgcaatatggaggcatttgcctctgcactgaggagaaaactatcttttccaagataggaggaaaggagataagtggaattaaagagaacctttgagcacagagttgggaaactgaaggtatttgtgttgtgctccctcaatcttttaattcaactataagctaaacccatgaaacttgagtagtttcagttatctgacttttttcttctcttttgatacagtgttggctattctgggtcttttgcctctctttatgtacttaagaatcagtttgccaatgtatgcaaaataactggctgggattttgattgtgattggcttgaatctatagatggagttgggaaggactgacatcttgacaatgttgaagcttcctattcatcattatgaaatatttctccatttgtttgattctttgatttcttttatcagaatttagttttcctcatatagtcttttaaaatattttgttatattttgttcaagtattttgtttttgaggaatgccaatgtaaatggtattgtgattttaatttcaaattccaatttttcattgctgttatataggaaaatgattttttttgcatgttagccttatatctttcaactttgctataatcaattattgatagtttcaaggattttttggtcaattattttgaatcttctacatagattatcatcatctgaacttagttttatttcttccttcccaatctgtatacctttatctccttttcttatttcattagctaggacttccagtatgatgttgaaagtagtggtgagaggggatatcttggtcttgttcttgatcttagtgggaaaacttcaagtttcttatcattaagtatgattttagctggagggtttttgtagaagttttttttttttaagttgaagaagtctccttctatttttagtttgctgatttttaaaaagaatcaggaatgggtgttaaattttgtgaaatgcttttctgcaactattgatttgagcactttatttttcttctttggcttgttgatgtgaagtacattaattgatttttgaatgctgaatcaaccttttgtacctgagattaatcccgtttggttgtggtatataattatttgtatacatgttgagttcgatttgctaatactttttgagaatttttgcattggtgttcatgaaaaaatattggtgtgtagttttttgtgacatctttatctgcttatggttttaaggtaatgctggcctcatagcatgagttagggagtatttcctctacttttacatttgagaagagattgcagagaattagtaaaattcctactttaaatattttgtggaattcaccagtgaacccatctggacctggtgctttctgttttggaaggtcattaattattttaaaatagatataggcctattcagattacctattttttctcatgcgagttttagcagattgtctttcaaggaattggtctatttcatttaggttatcaaatatgtcaacgtagagttattcatagtattcttttattatccttttaatgtgcaagggatctgtagtgatgtccccttttttgttttattgatattagcaatttgtgtcacatcttttattttgctttgttagccaggctagagatatctctatttttgatgtttttgatgaaccaactttttgttttattgattttctctgttgatttcgtgatttcaatttcatgatttttaaattatgcttacatttgatttaatttgatcttcttttgctagttatccaaggtggaagcttatattgttaagatccttttgcattcttatgcattcaatgatgtaaatttccctctaagcactgctttttctgcatctcacaaatattcatgagttgtattttcatgttcatttagtttgaaatatttttaaatttctcttgatatttctcttttgacccatgtgttacttagaagtgtgttgtttaatcaccatttttaaaaattttctagctatctttctgttattgatttctagtttaattccattgtggtctgagagcatatattgtataattttaatttttataaaatttgttaaggtgtgatttatggcccagaatgtggtctatcttggtgaatgttccatgtaagctttggaagactgtgtattctgctatatttgaatgaggtagtctatagacatcaattatgtccagttgattgatggtgctgttgaattcaactatgtccttactgattttccacctgctagatctgtccattctttgcagagggacactgaagtctccaactctagtagtgaatattctatttcttgttacagttttatcaacttctgcttcatgtcttttgatgctttgttgctagaaacatacacatgaagaattggtatgtcttttggagcatgacccatttatcctcatataatgcccctcattatttcctcgccctgatgtctgttctctctgaaagaaatatagcctctccaggtctcttttggttggtgttaaaatgacttaactttctttatcccccttacttttagtttatatgtggttttaaatttaaagtgggtttcttgtagacagcaaatagttcagagttgtttttcgatccactttgacaatctttgtcttttaattggtatatttggactattgatattttaagtgattattgatatagttagataaacatctactatatttattactgttttctgtctgttacactacttgttctttgtttatatttttattgtctactctttttctttccattgtggttttaatcgagcattttatatgtttccattttcttttcttagcatagtaattcttctttaaaaaaacattttttagtggttgcccctagagtttgcaatatacatttacaactaatctaagtccattttcaaataatactaaataatttcatgtgtagtgcaagtaccttttaataataaaacactcccagttccaccttccagtctcttgtattatagctataatttagttcacttacatatatgggtatacctaagtatatacattatcatatttatgattgaatatattgatgaaattattttgaaaaaactgttatcgttaaatcaattaagagtaagaaaaatagttctaattttattataaaatgaaataccttcatttattcattctctaatacactttctttctttatgtagatccaagtttctgacctgtataattttccttttctctcttcagcttctttgaacatttcttaccagccagacctactgacaacaattttccccaatttttgtttgtctgatagagactttatttcttcttgacttttgaagaataattccacagggcacagaactctagattggtgatttcttcccctcaaacccttaaatatttcattccactgccttcttgcttgcattgtttctgagaagttagatataattcttatctttgcctttctataggtaagatgttttttcctctggcttctatcaagattttttctttatgaacatgatatgcctttctttttgaacatgatatgcctttctttttgaacatgatatgcctttgtgtcggattttttttggcattattctgcttggttttctctgagtttcttggatatgtggtatggtatctgacactaatttggaaaaattctcagtcattattgcttcaaatatttcttctgttcttttttttcctttattctccttctggtattcccattacatgtatgttacagtttttgtagtcatcccgctgttttggatattctgtttttttcagtttttttttccttcgcatttcagtgttggaagtttctattgacatattctcaacctcagagattctttcttcagctgtgttcagtctaccaatgagtccatcaaaggcattttacatttttattacagaatttttgacctatagaatttcttttgattccatctttgaatctccatttctcttctgcttttcatctgttcttgcatgttgcctactttttccatgaaaacctttagctttttttttttttctttttgaggtggagtctcactgttgcccaggctggagtgcagtggtgtgatcttggctcactgcaacctctgcctcctgggttcaagtgattctcctcctcagcctcccaagtagctgggattacaggtgcctgccaccatgcctgagtaatttttgtatttttagtagagatggggttttatcatgttggccaggcgggtcttgaactcctaacctcaagtgatctgcccaccttagcctcccaaattgctgggattataggtgtgagccaccatgccctgcctttagcatgttaatcatagttgttttaaattcctgatctgttaattccaacatccctgtcatatctgactgtggttctgatgcttgctctgtgttttcaaatggtgtttttttttttttgccttttagtaagccttgtaattttttattgaaaggtggacatgatgtgctgggtaaaaggaactgtagtaaataggcctttagtaatgtactggtaggtgtagcagagggtgagggaagtattctgtagtcctatgattaggttttagtcttttagtgagcctgtgcgcctgcagcttggaagcacttgtgaagtgttttttcaccccttttggtgggacatagtgactagtgtgagcgggagttgagtatttcccttcccctaggtcagttaggctctgaaaaaaccctgataggttaggcatggtaaaatagtctcttttgagggcaggcattgttataagaatagaatgctctggggccaggtgcggtggctcacgcctgtaatccccgcactttgggaggctaaggcaggtggatcacctgaggtcaggagttcgagaccagcctggccaacatggtgaaaccccgtctctactaaaaatacaaaaatcagccaggtgtggtggcacacacctataatcccagctactcaggaggctgaggcaggagaactgcttgaacccagtaagtggaggttacagtgacccaagattgtgccactgcagtctagtctgggtgacagagcaagactccgtctcaaaaaaaaaagaatgctctggcatatttgaaaatggttacttttccctttttttctctgatcttcactgtgagaacctggtaagcatcctataggcaaaattcataaaagtatagaagtcggccagtgacttggacccacttggaattttcttgctctcacatcatgcacactgaatctccagcaatttttcacttacagtttaggttttcctaccctactactggttctctcagaggtttctgcttattggtttctgttttgtaagttgtgattctctgtacctaactgcctgtctcccattttggggggcagtggtttgccctgtgacctcacttctctgacagatctaagaaaagttgtttatttttcagtgtgctctgctttttacttgttacgatgaagccaaccactttcagaatttctacaaaccagatcagaatctggaagtcctgtttttttattttttttatccctttgtttagcatgttacctatcttaacacattttaaataagtgaatgcatagcttatatctacttctaggttatatgcttccttagaataggaattgattcttaaaatgtcgttctgctcacgcctgtaattccagcactttgggaggccaaggcaggcggatcacttggggtcaggagttcaagaccagcctggtcaacatggtaaaaccctgtgcctgcaaaaaatacaaaaattagctgggcatggtggtggccatctgtaatcccagctactagggaagctaaggcatgagaatcacttgaacctgggaggtggaggttgcagtgagctgagatcgcgccactgcactccagcctgggtgacaagagcaaaactccatctcataaataaataaataaataaataaataaataataaaaataaaaaaataaaataaaacaaaaattttattctgagcagtctctgaagaatataaattctactgccttgcctttagaacttataacagcatctcgcaaactatcacaagatgctccaaacatacttcttatgtgctgaattaagaagtcaactcaaatttagtatactagtaatatttttggatatcccaaaacactgccagctcagctttaggctgcccttcttgggggggaaaaaagcagttgaaatttaggacttaagtgggcatctcgtttaatttttaatggatttctatgttgttggttatggtgaagaggtgaaaagaataaatattctgtgcagaaaaattattcagtcttcatgtgaaaacactttgtccatagcaattactttatgaaaaagatgtggtattactttctttgctcttaactgagacctttaatttaaagaacctatactttacaagtttttattttcaatgcatgaaaaatgtagcagctatttcacaacctttacttttaaaatccatttttctttttaatctcaaatagttttttcttaaaaccttttgactttttatctaaattgtaatagccagagcaccttcccacaactagaatatctcatcctttttgtcttttctttttcctctcaaaatgcctactgggaacttaatttggagtcagattcttcatgataaatctggacttaatcaaaattcctcatatggtatattgtatatatcacagtactggatagtcctctgattaaatagatatttgatagtactttaaggtctatacttttggatgaacttaactgctttctccatttgtagtctcttgaaaatacagaaatttcagaaataatttataagaatatcaaggattcaaatcatatcagcacaaacacctaaatacttgtttgctttgttaaacacatatcccattttctatcttgataaacattggtgtaaagtagttgaatcattcagtgggtataagcagcatattctcaatactatgtttcattaataattaatagagatatatgaacacataaaagattcaattataatcaccttgtggatctaaatttcagttgacttgtcatcttgatttctggagaccacaaggtaatgaaaaataattacaagagtcttccatctgttgcagtattaaaatggcgagtaagacaccctgaaaggaaatgttctattcatggtacaatgcaattacagctagcaccaaattcaacactgtttaactttcaacatattattttgatttatcttgatccaacattctcagggaggaggtgcattgaagttattagaaaacactgacttagatttagggtatgtcttaaaagcttatttgcgggaagtactctagccttattcaacagatcactgagaagcctggaaaaacaaatcccggaaactaattattatgtgccagttatataaacaagaagactttgttgggtacaaaccagtgattccttgcctttgaaaaatgtgtcagatatcatgcattaccagcagttcaatgatataaggaaaccagagtaatagctaaaacctttaaagctaaaccaaagatttacaaattgcctcttcatccagtctttcccaacctaaaaactgagttctctaaaaattttagtatttttttctgaagaaaagggaacatggacatttatctaatcctcattagaaatctgactaatgataacaaggatttagacctcaagcacttcttaccaaaattcttgatatgaccttatagcaaattactttcacctgttgaactttcctttcttttattcccctgtacctcacctgcactgggcatattcaagttgcttatacaacactttactattgtgttagaaaaatcatgacacatgatgaatgtgtttgtgcaacatgagctgattcataaatgaaaatgtgcattgaaattccacaatattttaaaattaggagtttatctagcaattgaacaaaattgattaaatccattatttgttagatcagctaaattacataagttcattcatctgctcataaatccatccattcttccatctggctatcccttagtcaattcaaataaatatttatggggcactttgggtaagccaggtgctaagaattcaatgcaaaacaagatagactcccctgtccttgttgaacttatatttttggtacaaacaaaagcaataatcaagaaaaaataaaaaaagtactgattgtgattaataatatgaagaaattcaacagagtattgtacttaacatttgattgatctgattttctcagttgtctgagaacaaacatttgtgaaaatctcattgtagagttcttacgatggatagggggtcaactgtgtcattattgcttatcagcttatcccaaagacctagtttattaccagattgcaaatagtgttcaataaattattcttattaagggttgttatgtactctaaaacatttattgtggtcccttcactggttctggtttacaaacttacttttctatgatgacatagtatagaaattgagagtgaatatttagaagttcatttttattatatatttttgaagtattgatatgtagtgaattagaaatttaaaaagaaaacaaaactgtccttcactacagattgaaaagcattatactaaaagaccatttgctcagttatagtatataaaggccaaatgacttaaaaacaaattatgtaaggagaaggaaacaaccatttattcagtgccactaactgtcagccagttttttcagtggtcagttaatgactgcagtagtgttctaccttgctcaaagcaccctcctcaagttctggcatctaagctgacatcagaacacagagttggggctctctgtgggtcacctctagcacttgatctcctcatgcagtgcatggtgctctcacgtctatgctatgttcttatggtctttaggtaacaagaataattttctttcttttccttactatacattttgctttctgaaattcccttctcgccaatccaggtgaatgtcagaatgtgatttgacaactgtccaaagtactcattcactgaggagtggtaaggccttcgcccaacctgccttctctgggaatatactgctgcctgaacatatcattgtttattgccaggcttgaacttcaccaaattaatttattagggtcaacatctaaatattagaactatttcagattaatttttaagtcgtatccactttgggtactagatcaaattgcaggtctctgcttctggcttgagcctatgtttagagatgatgtgcatgaagacactctttgcttttcctttatgcaaaatgggcattttcaatctttttgtcattagtaaaggtcagtgataaaggaagtctgcatcaggggtccaattccttatggccagtttctctattctgttccaaggttgtttgtctccatatatcaacattggtcaggattgaaagtgtgcaacaaggtttgaatgaataagtgaaaatcttccactggtgacaggataaaatattccaatggtttttattgaagtacaatactgaattatgtttatggcatggtacctatatgtcacagaagtgatcccatcacttttaccttatag

SEQ ID NO: 71 - DNA, 14667 bp, Homo sapiens
  misc_feature (1)..(14667): CFTR exon 19 containing 3849 + 10 kb C-to-T mutation
gtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatataatgggtttattttaaatgtgattgtacttgcagaatatctaattaattgctaggttaataactaaagaagccattaaataaatcaaaattgtaacatgttttagatttcccatcttgaaaatgtcttccaaaaatatcttattgctgactccatctattgtcttaaattttatctaagttccattctgccaaacaagtgatactttttttctagcttttttcagtttgtttgttttgtttttctttgaagttttaattcagacatagattattttttcccagttatttactatatttattaagcatgagtaattgacattattttgaaatccttcttatggatcccagcactgggctgaacacatagaaggaacttaatatatactgatttctggaattgattcttggagacagggatggtcattatccatatacttcaggctccataaacatatttcttaattgccttcaaatccctattctggactgctctataaatctagacaagagtattatatattttgattgatattttttagataaaataaaagggagctgaaaactgaattgcaaactgaattttaaaactttatctctctgtggttaattgcaaacacagatacaaaaatatagagagagatacagttagtaaagatgttaggtcaccgttactaacactgacatagaaacagttttgctcatgagtttcagaatatatgagtttgattttgcccatggattttagaatatttgataaacatttaatgcattgtacaaattctgtgaaaacatatatataggatgtgcgaaaagtccctgtgtatcatgtgaaatggcttaaaacagaacaccataggtattcatatcagtgaataccataggtagctgaaagtgttttttcctggggtcgccaagatgaatgccaaaagtgatatcattattataaacaatagccagaataggttggtataaacctggtagaaagccttgataaattgactttctctcctcctgacatcctgccacccctttgctttgctgatgctcatttgtccactaaattaaactcaagcaagccctagtaaagtaatagaatttgtggagtcctcattagtataggaagtttccctgatgtgagattagtaattagagatgtagcaaaatgagaaagaagtaatatgcttagatatttcattttctctgaacctgtatatacaaaataggccatgcgtgttcagtaactattcactgcaaggcactctctaggtactttgggggaattggaaattactcacataaggctatggattgtgccatttgtcaaaagacaaaatgacaacaaatttagtttaaagacctcagtcagctttattttctattctagatttggacagtccttcatttcacaaattggagtaagtgttccaataagttgagcaaaggagcttggctttatagacccaaaaaaagggccaaaggaagcagaaacaaagaacaataagagaattggtcatttcaaagttacttttcttgaaaggtggggacaaggagacagaataatagaaaagtcactgattggttaacattggattaagaattaaaacagaggaaactttaagattgaagtttgaaactgacttgtttgggaaatcaggctgtcttctttcttgatttcttagaaggccggataacaactgagttttgctttggtgaacatgggtgactccatttttacttttagtctggtctgttgaggcctcgtgagagagcttaatctaaaacaatgacttcctataatttttgtttgacacatccaaagagggactctaatatttattgagagcttatcatatcttaagtactgtttaaacacttttatttgctattacatttgatcttattataactctaaaggcagaaatgattgcttttattttccacaatggaggaaactgaggttcaattaagtgagtaaggaagcagggatcttaaacccagataccattgctcctctttaaaggtggaagaacagaaaacatggggcaggggaagagagaaagtttctgtcccaggacatgataatctaaaagggaaaacgtaagatccactgaaacctgaggcagatttattgtggcaataacaaagcttaagtttcacagaccttcatttgcctgagccaactttgaaggccatgtatctaattttgtttttataattctataatctttattcttgaaaagagccctccctccaaatttacaagctttgggcccccaaaatccttgaaatgcccttgaataagagatatccaggtaaatgctatgggaattcagaggaggaagcagttagtatcagttggcggagagttaggctattaagagaaggttttatataggaagtggcatttagaatgaagctttgagaactgagctgtgtatttgaacaagtaaaggtggtgttgcagaattttgctccttagttctattaaaaacccgggttcttgtcacatgatccggaaaatttaggcacacagatacattgaagcatgagtagagcaggattttattgggcaaaaaggaaaaaaagaaaactcagcaaatcgagatggagtcttgctcacagattgaatcccaggccaccacaaaggaactgaagagatcgggcttctcccctgcataaggtgcaaattccccatggctccacccacttccccttagtgtgcatgtggggctccagtccacggtgggcatgcccagacaagccttgggcaggttccctcatctgtgcaaaagcatctgatgtaaacacttgaggggtggttcggagattctctgggacccttttattttcttatctgcctaggcatttggctgtctcagtgggtgggaaagggtgctccaggcaaagggcataacatgaggcaaagggcatgcacagaaaacagtgactggttcagtcaggttgggggatgccaaaggaagtaatgggagacaagattggagcaagatagataagagattgtggattttttttcttttttatctatataaatacagagacagggtctcactatgttgcccaggctggtctcaaactcctggcctcaagtgatcctcccacctcatcctcccaaagtgctaggattacaggcatgaggcactgtgcccaacctccaattttggattttgagagctaaagcaatatagtcgaaaactcagataatccaggtagattttgctattaggtgctatttggttcctggtacagagctaaaacccttggaatttcctaagtgataagagctacaggagcatcttttgttatatgtttccccccctagttcctgaaatagctctagagaaatacaggtgaataacatcctttgttattcatatcaagcccctatcaaccataccccagtttctatttatgaagtggcttttgggaagtccctaaagacaggagtggggaaaggctggttgtcagggggatgggttgaaactttcatcttccccccttgacctccagggagggatgagtggctgaaaattgtgtaaaatcaacaatggccagtgatttaatcaaccatgcctatgtaatgaagccacccgataagccttaactggaactttttggagagcctccaggctggtgaagacattgaggtgctcagaaggtggtattccagagagagcacagaatctctgttccccttcccacattcattttgctatgcatctctcccatctggctgttcttgagaggtatccgtttataataaactggtaacctagtaagtaaactgttaccctgagttctgtgagccattctagcaaattatcaaacctaaagagttcatggatacgtgcaatttacagatgcacagtcagaagcacagatgacaatctgggcttgccattggcatttgaagtgtgttgggaggcagtcttacaggaatgagcccttatcctgtggggtctatgctaataacagacagttgtcagcattgcttggtgtcgaaaacccacattgttggtgtcagaagtattgtcagtaggatagggaaaacagtttgttttctttttttagtggtctttggtcatctttaagagcagggcttctcaaagtgtggtccttgaaccagcatcacctgtaccacgtaagaacttatgagaaatgttcattcttgggccccaacaaagaattaaaaattctgagggtgtgaacggggtctgagtttcagcacaacttcccgaccatgctgatgcattcttgcccaagcatgaaagccctcccttgtttaagaaggccattagggccgggtgtggtggctcatgcttgtaatcgagcactttgagaggacatagtgggaggatcacttgagccctggagttctagacaagcctgggcaacatggcaaaatgctgtctccacaaaaatcacaaaaattaggtgggcgtgtgttgtgtgcctataggcccagctacttaggagactgaggcaggaggatcgcttgagcccaggagattaaggctgcagcgagctgtgatggcaccactacagcctggatgacagagtgagacactgtctcaaaaaaaaaaaagaaaaagaaaaagaaaaaagaaaggaaaatgaaaaagaacgccattaggtataaaggagcaatggtaaaagaccagttgcaaaaggttagggaatgggtggttactgaaataagaagctatgtagaacactagtgttggtggcaggaagtagaaagcaagagcactgctctgtgggggatggtcatagcaaatgcaatatggaggcatttgcctctgcactgaggagaaaactatcttttccaagataggaggaaaggagataagtggaattaaagagaacctttgagcacagagttgggaaactgaaggtatttgtgttgtgctccctcaatcttttaattcaactataagctaaacccatgaaacttgagtagtttcagttatctgacttttttcttctcttttgatacagtgttggctattctgggtcttttgcctctctttatgtacttaagaatcagtttgccaatgtatgcaaaataactggctgggattttgattgtgattggcttgaatctatagatggagttgggaaggactgacatcttgacaatgttgaagcttcctattcatcattatgaaatatttctccatttgtttgattctttgatttcttttatcagaatttagttttcctcatatagtcttttaaaatattttgttatattttgttcaagtattttgtttttgaggaatgccaatgtaaatggtattgtgattttaatttcaaattccaatttttcattgctgttatataggaaaatgattttttttgcatgttagccttatatctttcaactttgctataatcaattattgatagtttcaaggattttttggtcaattattttgaatcttctacatagattatcatcatctgaacttagttttatttcttccttcccaatctgtatacctttatctccttttcttatttcattagctaggacttccagtatgatgttgaaagtagtggtgagaggggatatcttggtcttgttcttgatcttagtgggaaaacttcaagtttcttatcattaagtatgattttagctggagggtttttgtagaagttttttttttttaagttgaagaagtctccttctatttttagtttgctgatttttaaaaagaatcaggaatgggtgttaaattttgtgaaatgcttttctgcaactattgatttgagcactttatttttcttctttggcttgttgatgtgaagtacattaattgatttttgaatgctgaatcaaccttttgtacctgagattaatcccgtttggttgtggtatataattatttgtatacatgttgagttcgatttgctaatactttttgagaatttttgcattggtgttcatgaaaaaatattggtgtgtagttttttgtgacatctttatctgcttatggttttaaggtaatgctggcctcatagcatgagttagggagtatttcctctacttttacatttgagaagagattgcagagaattagtaaaattcctactttaaatattttgtggaattcaccagtgaacccatctggacctggtgctttctgttttggaaggtcattaattattttaaaatagatataggcctattcagattacctattttttctcatgcgagttttagcagattgtctttcaaggaattggtctatttcatttaggttatcaaatatgtcaacgtagagttattcatagtattcttttattatccttttaatgtgcaagggatctgtagtgatgtccccttttttgttttattgatattagcaatttgtgtcacatcttttattttgctttgttagccaggctagagatatctctatttttgatgtttttgatgaaccaactttttgttttattgattttctctgttgatttcgtgatttcaatttcatgatttttaaattatgcttacatttgatttaatttgatcttcttttgctagttatccaaggtggaagcttatattgttaagatccttttgcattcttatgcattcaatgatgtaaatttccctctaagcactgctttttctgcatctcacaaatattcatgagttgtattttcatgttcatttagtttgaaatatttttaaatttctcttgatatttctcttttgacccatgtgttacttagaagtgtgttgtttaatcaccatttttaaaaattttctagctatctttctgttattgatttctagtttaattccattgtggtctgagagcatatattgtataattttaatttttataaaatttgttaaggtgtgatttatggcccagaatgtggtctatcttggtgaatgttccatgtaagctttggaagactgtgtattctgctatatttgaatgaggtagtctatagacatcaattatgtccagttgattgatggtgctgttgaattcaactatgtccttactgattttccacctgctagatctgtccattctttgcagagggacactgaagtctccaactctagtagtgaatattctatttcttgttacagttttatcaacttctgcttcatgtcttttgatgctttgttgctagaaacatacacatgaagaattggtatgtcttttggagcatgacccatttatcctcatataatgcccctcattatttcctcgccctgatgtctgttctctctgaaagaaatatagcctctccaggtctcttttggttggtgttaaaatgacttaactttctttatcccccttacttttagtttatatgtggttttaaatttaaagtgggtttcttgtagacagcaaatagttcagagttgtttttcgatccactttgacaatctttgtcttttaattggtatatttggactattgatattttaagtgattattgatatagttagataaacatctactatatttattactgttttctgtctgttacactacttgttctttgtttatatttttattgtctactctttttctttccattgtggttttaatcgagcattttatatgtttccattttcttttcttagcatagtaattcttctttaaaaaaacattttttagtggttgcccctagagtttgcaatatacatttacaactaatctaagtccattttcaaataatactaaataatttcatgtgtagtgcaagtaccttttaataataaaacactcccagttccaccttccagtctcttgtattatagctataatttagttcacttacatatatgggtatacctaagtatatacattatcatatttatgattgaatatattgatgaaattattttgaaaaaactgttatcgttaaatcaattaagagtaagaaaaatagttctaattttattataaaatgaaataccttcatttattcattctctaatacactttctttctttatgtagatccaagtttctgacctgtataattttccttttctctcttcagcttctttgaacatttcttaccagccagacctactgacaacaattttccccaatttttgtttgtctgatagagactttatttcttcttgacttttgaagaataattccacagggcacagaactctagattggtgatttcttcccctcaaacccttaaatatttcattccactgccttcttgcttgcattgtttctgagaagttagatataattcttatctttgcctttctataggtaagatgttttttcctctggcttctatcaagattttttctttatgaacatgatatgcctttctttttgaacatgatatgcctttctttttgaacatgatatgcctttgtgtcggattttttttggcattattctgcttggttttctctgagtttcttggatatgtggtatggtatctgacactaatttggaaaaattctcagtcattattgcttcaaatatttcttctgttcttttttttcctttattctccttctggtattcccattacatgtatgttacagtttttgtagtcatcccgctgttttggatattctgtttttttcagtttttttttccttcgcatttcagtgttggaagtttctattgacatattctcaacctcagagattctttcttcagctgtgttcagtctaccaatgagtccatcaaaggcattttacatttttattacagaatttttgacctatagaatttcttttgattccatctttgaatctccatttctcttctgcttttcatctgttcttgcatgttgcctactttttccatgaaaacctttagctttttttttttttctttttgaggtggagtctcactgttgcccaggctggagtgcagtggtgtgatcttggctcactgcaacctctgcctcctgggttcaagtgattctcctcctcagcctcccaagtagctgggattacaggtgcctgccaccatgcctgagtaatttttgtatttttagtagagatggggttttatcatgttggccaggcgggtcttgaactcctaacctcaagtgatctgcccaccttagcctcccaaattgctgggattataggtgtgagccaccatgccctgcctttagcatgttaatcatagttgttttaaattcctgatctgttaattccaacatccctgtcatatctgactgtggttctgatgcttgctctgtgttttcaaatggtgtttttttttttttgccttttagtaagccttgtaattttttattgaaaggtggacatgatgtgctgggtaaaaggaactgtagtaaataggcctttagtaatgtactggtaggtgtagcagagggtgagggaagtattctgtagtcctatgattaggttttagtcttttagtgagcctgtgcgcctgcagcttggaagcacttgtgaagtgttttttcaccccttttggtgggacatagtgactagtgtgagcgggagttgagtatttcccttcccctaggtcagttaggctctgaaaaaaccctgataggttaggcatggtaaaatagtctcttttgagggcaggcattgttataagaatagaatgctctggggccaggtgcggtggctcacgcctgtaatccccgcactttgggaggctaaggcaggtggatcacctgaggtcaggagttcgagaccagcctggccaacatggtgaaaccccgtctctactaaaaatacaaaaatcagccaggtgtggtggcacacacctataatcccagctactcaggaggctgaggcaggagaactgcttgaacccagtaagtggaggttacagtgacccaagattgtgccactgcagtctagtctgggtgacagagcaagactccgtctcaaaaaaaaaagaatgctctggcatatttgaaaatggttacttttccctttttttctctgatcttcactgtgagaacctggtaagcatcctataggcaaaattcataaaagtatagaagtcggccagtgacttggacccacttggaattttcttgctctcacatcatgcacactgaatctccagcaatttttcacttacagtttaggttttcctaccctactactggttctctcagaggtttctgcttattggtttctgttttgtaagttgtgattctctgtacctaactgcctgtctcccattttggggggcagtggtttgccctgtgacctcacttctctgacagatctaagaaaagttgtttatttttcagtgtgctctgctttttacttgttacgatgaagccaaccactttcagaatttctacaaaccagatcagaatctggaagtcctgtttttttattttttttatccctttgtttagcatgttacctatcttaacacattttaaataagtgaatgcatagcttatatctacttctaggttatatgcttccttagaataggaattgattcttaaaatgtcgttctgctcacgcctgtaattccagcactttgggaggccaaggcaggcggatcacttggggtcaggagttcaagaccagcctggtcaacatggtaaaaccctgtgcctgcaaaaaatacaaaaattagctgggcatggtggtggccatctgtaatcccagctactagggaagctaaggcatgagaatcacttgaacctgggaggtggaggttgcagtgagctgagatcgcgccactgcactccagcctgggtgacaagagcaaaactccatctcataaataaataaataaataaataaataaataataaaaataaaaaaataaaataaaacaaaaattttattctgagcagtctctgaagaatataaattctactgccttgcctttagaacttataacagcatctcgcaaactatcacaagatgctccaaacatacttcttatgtgctgaattaagaagtcaactcaaatttagtatactagtaatatttttggatatcccaaaacactgccagctcagctttaggctgcccttcttgggggggaaaaaagcagttgaaatttaggacttaagtgggcatctcgtttaatttttaatggatttctatgttgttggttatggtgaagaggtgaaaagaataaatattctgtgcagaaaaattattcagtcttcatgtgaaaacactttgtccatagcaattactttatgaaaaagatgtggtattactttctttgctcttaactgagacctttaatttaaagaacctatactttacaagtttttattttcaatgcatgaaaaatgtagcagctatttcacaacctttacttttaaaatccatttttctttttaatctcaaatagttttttcttaaaaccttttgactttttatctaaattgtaatagccagagcaccttcccacaactagaatatctcatcctttttgtcttttctttttcctctcaaaatgcctactgggaacttaatttggagtcagattcttcatgataaatctggacttaatcaaaattcctcatatggtatattgtatatatcacagtactggatagtcctctgattaaatagatatttgatagtactttaaggtctatacttttggatgaacttaactgctttctccatttgtagtctcttgaaaatacagaaatttcagaaataatttataagaatatcaaggattcaaatcatatcagcacaaacacctaaatacttgtttgctttgttaaacacatatcccattttctatcttgataaacattggtgtaaagtagttgaatcattcagtgggtataagcagcatattctcaatactatgtttcattaataattaatagagatatatgaacacataaaagattcaattataatcaccttgtggatctaaatttcagttgacttgtcatcttgatttctggagaccacaaggtaatgaaaaataattacaagagtcttccatctgttgcagtattaaaatggtgagtaagacaccctgaaaggaaatgttctattcatggtacaatgcaattacagctagcaccaaattcaacactgtttaactttcaacatattattttgatttatcttgatccaacattctcagggaggaggtgcattgaagttattagaaaacactgacttagatttagggtatgtcttaaaagcttatttgcgggaagtactctagccttattcaacagatcactgagaagcctggaaaaacaaatcccggaaactaattattatgtgccagttatataaacaagaagactttgttgggtacaaaccagtgattccttgcctttgaaaaatgtgtcagatatcatgcattaccagcagttcaatgatataaggaaaccagagtaatagctaaaacctttaaagctaaaccaaagatttacaaattgcctcttcatccagtctttcccaacctaaaaactgagttctctaaaaattttagtatttttttctgaagaaaagggaacatggacatttatctaatcctcattagaaatctgactaatgataacaaggatttagacctcaagcacttcttaccaaaattcttgatatgaccttatagcaaattactttcacctgttgaactttcctttcttttattcccctgtacctcacctgcactgggcatattcaagttgcttatacaacactttactattgtgttagaaaaatcatgacacatgatgaatgtgtttgtgcaacatgagctgattcataaatgaaaatgtgcattgaaattccacaatattttaaaattaggagtttatctagcaattgaacaaaattgattaaatccattatttgttagatcagctaaattacataagttcattcatctgctcataaatccatccattcttccatctggctatcccttagtcaattcaaataaatatttatggggcactttgggtaagccaggtgctaagaattcaatgcaaaacaagatagactcccctgtccttgttgaacttatatttttggtacaaacaaaagcaataatcaagaaaaaataaaaaaagtactgattgtgattaataatatgaagaaattcaacagagtattgtacttaacatttgattgatctgattttctcagttgtctgagaacaaacatttgtgaaaatctcattgtagagttcttacgatggatagggggtcaactgtgtcattattgcttatcagcttatcccaaagacctagtttattaccagattgcaaatagtgttcaataaattattcttattaagggttgttatgtactctaaaacatttattgtggtcccttcactggttctggtttacaaacttacttttctatgatgacatagtatagaaattgagagtgaatatttagaagttcatttttattatatatttttgaagtattgatatgtagtgaattagaaatttaaaaagaaaacaaaactgtccttcactacagattgaaaagcattatactaaaagaccatttgctcagttatagtatataaaggccaaatgacttaaaaacaaattatgtaaggagaaggaaacaaccatttattcagtgccactaactgtcagccagttttttcagtggtcagttaatgactgcagtagtgttctaccttgctcaaagcaccctcctcaagttctggcatctaagctgacatcagaacacagagttggggctctctgtgggtcacctctagcacttgatctcctcatgcagtgcatggtgctctcacgtctatgctatgttcttatggtctttaggtaacaagaataattttctttcttttccttactatacattttgctttctgaaattcccttctcgccaatccaggtgaatgtcagaatgtgatttgacaactgtccaaagtactcattcactgaggagtggtaaggccttcgcccaacctgccttctctgggaatatactgctgcctgaacatatcattgtttattgccaggcttgaacttcaccaaattaatttattagggtcaacatctaaatattagaactatttcagattaatttttaagtcgtatccactttgggtactagatcaaattgcaggtctctgcttctggcttgagcctatgtttagagatgatgtgcatgaagacactctttgcttttcctttatgcaaaatgggcattttcaatctttttgtcattagtaaaggtcagtgataaaggaagtctgcatcaggggtccaattccttatggccagtttctctattctgttccaaggttgtttgtctccatatatcaacattggtcaggattgaaagtgtgcaacaaggtttgaatgaataagtgaaaatcttccactggtgacaggataaaatattccaatggtttttattgaagtacaatactgaattatgtttatggcatggtacctatatgtcacagaagtgatcccatcacttttaccttatag

SEQ ID NO: 72 - DNA, 18 bp, Artificial
  Synthetic oligonucleotide
gtcttactcgccatttta

SEQ ID NO: 73 - DNA, 18 bp, Artificial
  Synthetic oligonucleotide
gtcttactcaccatttta

SEQ ID NO: 74 - DNA, 3733 bp, Mus musculus
  misc_feature (1)..(3733): wild-type Mus musculus dystrophin intron 22, exon 23 and intron 23 sequences
gtctgtggacatttgaatatcataaataacaaagaacatgtcttatcagtcaagagatcatattgatatattaaacttaaggtaataatgaaaaagtaaagataataatgaaaaatcatagattatgagttggaaaaataaacagaacaatttgaccaaaaacatgactttttcttatttttttctatatattattttataaatatacagacataaatagatatatatttttaaattaaaagtactgtattaaaggaaaggtataatttcatttcatatttagtgacataagatatgaagtatgattattaaaattaaatcacattattttattataattactttatttttaattcctaatttctttaagcttaggtaaaatcaatggatttatataattagttagaatttaaatattaacaaactataacactatgattaaatgcttgatattgagtagttattttaatagcctaagtctggaaattaaatactagtaagagaaacttctgtgatgtgaggacatataaagactaatttttttgttgattctaaaaatcccatgttgtatacttattctttttaaatctgaaaatatattaatcatatattgcctaaatgtcttaataatgtttcactgtaggtaagttaaaatgtatcacatatataataaacatagttattaatgcatagatattcagtaaaattatgacttctaaatttctgtctaaatataatatgccctgtaatataatagaaattattcataagaatacatatatattgctttatcagatattctactttgtttagatctctaaattacataaacttttatttaccttcttcttgatatgaatgaaactcatcaaatatgcgtgttagtgtaaatgaacttctatttaattttgaggctctgcaaagttctttgaaagagcaacaaaatggcttcaactatctgagtgacactgtgaaggagatggccaagaaagcaccttcagaaatatgccagaaatatctgtcagaatttgaagagattgaggggcactggaagaaactttcctcccagttggtggaaagctgccaaaagctagaagaacatatgaataaacttcgaaaatttcaggtaagccgaggtttggcctttaaactatattttttcacatagcaattaattggaaaatgtgatgggaaacagatattttacccagagtccttcaaagatattgatgatatcaaaagccaaatctatttcaaaggattgcaacttgcctatttttcctatgaaaacagtaatgtgtcataccttcttggattgtctgtataaatgaattgattttttttcaccaactccaagtatacttaacattttaacataataatttaaaatatccttattccattatgttcattttttaagttgtagatatgatttagctcacagcatacatatatacacatgtattacatatgcatatattatatatatggcagacatatgttttcactaccatatttcacttttgaattatgaatatatgtttaatttctgccatatttccttccctacattgacttctattaatttagtatttcagtagttctaacacattaataataacctagactcaatacagtaatctaacaattatatttgtgcctgtaattctaagttagttaaattcataggttgtgtttctcatagttggccatttgtgaaatataataatatccgaaaagaaagttcaaaaatgtcatgacttcatatagagttattgaaacagtgcccttactttcattctggccatgctagtgacttgatcattcttgtattttacagctaaaacactaccaaaagtgtcaaatccatgatctacatgtttgactgaggctagcagcacttattccacccttatatgaagcctttaagagaaagtatatttgtttgctatttttaacttcttgaaggaacatacaatctttgtttcaagagctcatcctctttcatgctagtaaattttggtggcattgcatccatgtctgactctgaatctgtttctgtctatcctgctccctaacactgtaccatcttcctttttgaaaaaaaaatattgaattattttatttatttactttccaaagttgctcctgcctgttcctccttctccaagttcttcagtcccccctgctccccaccgatgagagggaaaggtcctgaattcactgggctccatgggggtccttttgcattttcttaaccttcttaataaaataggccttctagaattatatcatatacattgtgatatgacaaatgataaagtatattgttcagagttttaccttgttcatatttgcaatgtccccctgtcatgctggatattctttgattgggtatatttgctaacagattaagtatatttatcttcgttaagcagtataacttattaagaaagaactctattaatatgagaaataactaatgaaacaccactccacaggtgatttcagccactttatgaactgctggaagcaaaaatgagatctttgcaacatgaagcagttgctcagttcattaaactgtgttcaatatttcagccataacatacattagagaatgatttatattgttcaaacatttggtgctctatttttgcatgacgtgggattaaacacagcaccaacaatcaaacaattgcaaagatgtattacaagtattttttctttttaaaacaggaaagtatacttatatttccattgtccaaaccatcatgaaagggatagagattactgacacaaatttagagaaaggatttgagtggagtaagaattaaatgaaccaaagaagaattaatgtattcatcaagaagtcatggaggtgaaattggccttgaatgataccactaaggagagaatgttgagatccttatatttagtcaattgtttttaaatctgtagttattaaccacattttaatcatattgaaagggaaattttctgtgatgcatgtattttcaatataaattttagaaaagaagacaattataacttgattttgtgaattacatggaactaaagaaatgacagatttacatttgaaaattgactgaactaaagtacataaataaaagtcatacagaaaaatgtgggaggtgcttgtccatttataaaggacaaaaatgccatttgttgcctaatcattatttcttattggtcagaccaataagaaatcaagagctttgactttaaaggtaagaaaatcttaccttaaaatccccaactgaagggactgtttaaactgtcaactgcagaaaacaagttatggaagttcaggtttagggaaactataaacacaccataacattgagtttatgtgcatagtttgttttatgtacagtgagagtaaattgttagtattatcatgagttgttttgaaacttcaaatttctctagaggggtatgatttaatgttctcaagaggaacataataaaaccatatctggtattagtttttatttttaacaatagcagacttcatacaccaatgttcacagtgtagaccataaaatgcagtcttagtaaaaatattattctctataaagctacaatgagacctccctcaaacatacattgtttttttttttctaacttatgtttggatatatcatcatgatgaactatgttaaaaacaatcagagcttagtaatactttcatattgcttttttattccag

SEQ ID NO: 75 - DNA, 3733 bp, Mus musculus
  misc_feature (1)..(3733): mdx Mus musculus dystrophin intron 22, exon 23 and intron 23 sequences
gtctgtggacatttgaatatcataaataacaaagaacatgtcttatcagtcaagagatcatattgatatattaaacttaaggtaataatgaaaaagtaaagataataatgaaaaatcatagattatgagttggaaaaataaacagaacaatttgaccaaaaacatgactttttcttatttttttctatatattattttataaatatacagacataaatagatatatatttttaaattaaaagtactgtattaaaggaaaggtataatttcatttcatatttagtgacataagatatgaagtatgattattaaaattaaatcacattattttattataattactttatttttaattcctaatttctttaagcttaggtaaaatcaatggatttatataattagttagaatttaaatattaacaaactataacactatgattaaatgcttgatattgagtagttattttaatagcctaagtctggaaattaaatactagtaagagaaacttctgtgatgtgaggacatataaagactaatttttttgttgattctaaaaatcccatgttgtatacttattctttttaaatctgaaaatatattaatcatatattgcctaaatgtcttaataatgtttcactgtaggtaagttaaaatgtatcacatatataataaacatagttattaatgcatagatattcagtaaaattatgacttctaaatttctgtctaaatataatatgccctgtaatataatagaaattattcataagaatacatatatattgctttatcagatattctactttgtttagatctctaaattacataaacttttatttaccttcttcttgatatgaatgaaactcatcaaatatgcgtgttagtgtaaatgaacttctatttaattttgaggctctgcaaagttctttgaaagagcaataaaatggcttcaactatctgagtgacactgtgaaggagatggccaagaaagcaccttcagaaatatgccagaaatatctgtcagaatttgaagagattgaggggcactggaagaaactttcctcccagttggtggaaagctgccaaaagctagaagaacatatgaataaacttcgaaaatttcaggtaagccgaggtttggcctttaaactatattttttcacatagcaattaattggaaaatgtgatgggaaacagatattttacccagagtccttcaaagatattgatgatatcaaaagccaaatctatttcaaaggattgcaacttgcctatttttcctatgaaaacagtaatgtgtcataccttcttggattgtctgtataaatgaattgattttttttcaccaactccaagtatacttaacattttaacataataatttaaaatatccttattccattatgttcattttttaagttgtagatatgatttagctcacagcatacatatatacacatgtattacatatgcatatattatatatatggcagacatatgttttcactaccatatttcacttttgaattatgaatatatgtttaatttctgccatatttccttccctacattgacttctattaatttagtatttcagtagttctaacacattaataataacctagactcaatacagtaatctaacaattatatttgtgcctgtaattctaagttagttaaattcataggttgtgtttctcatagttggccatttgtgaaatataataatatccgaaaagaaagttcaaaaatgtcatgacttcatatagagttattgaaacagtgcccttactttcattctggccatgctagtgacttgatcattcttgtattttacagctaaaacactaccaaaagtgtcaaatccatgatctacatgtttgactgaggctagcagcacttattccacccttatatgaagcctttaagagaaagtatatttgtttgctatttttaacttcttgaaggaacatacaatctttgtttcaagagctcatcctctttcatgctagtaaattttggtggcattgcatccatgtctgactctgaatctgtttctgtctatcctgctccctaacactgtaccatcttcctttttgaaaaaaaaatattgaattattttatttatttactttccaaagttgctcctgcctgttcctccttctccaagttcttcagtcccccctgctccccaccgatgagagggaaaggtcctgaattcactgggctccatgggggtccttttgcattttcttaaccttcttaataaaataggccttctagaattatatcatatacattgtgatatgacaaatgataaagtatattgttcagagttttaccttgttcatatttgcaatgtccccctgtcatgctggatattctttgattgggtatatttgctaacagattaagtatatttatcttcgttaagcagtataacttattaagaaagaactctattaatatgagaaataactaatgaaacaccactccacaggtgatttcagccactttatgaactgctggaagcaaaaatgagatctttgcaacatgaagcagttgctcagttcattaaactgtgttcaatatttcagccataacatacattagagaatgatttatattgttcaaacatttggtgctctatttttgcatgacgtgggattaaacacagcaccaacaatcaaacaattgcaaagatgtattacaagtattttttctttttaaaacaggaaagtatacttatatttccattgtccaaaccatcatgaaagggatagagattactgacacaaatttagagaaaggatttgagtggagtaagaattaaatgaaccaaagaagaattaatgtattcatcaagaagtcatggaggtgaaattggccttgaatgataccactaaggagagaatgttgagatccttatatttagtcaattgtttttaaatctgtagttattaaccacattttaatcatattgaaagggaaattttctgtgatgcatgtattttcaatataaattttagaaaagaagacaattataacttgattttgtgaattacatggaactaaagaaatgacagatttacatttgaaaattgactgaactaaagtacataaataaaagtcatacagaaaaatgtgggaggtgcttgtccatttataaaggacaaaaatgccatttgttgcctaatcattatttcttattggtcagaccaataagaaatcaagagctttgactttaaaggtaagaaaatcttaccttaaaatccccaactgaagggactgtttaaactgtcaactgcagaaaacaagttatggaagttcaggtttagggaaactataaacacaccataacattgagtttatgtgcatagtttgttttatgtacagtgagagtaaattgttagtattatcatgagttgttttgaaacttcaaatttctctagaggggtatgatttaatgttctcaagaggaacataataaaaccatatctggtattagtttttatttttaacaatagcagacttcatacaccaatgttcacagtgtagaccataaaatgcagtcttagtaaaaatattattctctataaagctacaatgagacctccctcaaacatacattgtttttttttttctaacttatgtttggatatatcatcatgatgaactatgttaaaaacaatcagagcttagtaatactttcatattgcttttttattccag

SEQ ID NO: 76 - DNA, 25 bp, Artificial
  Synthetic oligonucleotide
aacctcggcttacctgaaattttcg

SEQ ID NO: 77 - DNA, 1653 bp, Hotaria parvula
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 78 - DNA, 17578 bp, Homo sapiens
  Intron (1)..(13645): intron 9
gtgagagtggctggctgcgcgtggaggtgtggggggctgcgcctggaggggtagggctgtgcctggaagggtagggctgcgcctggaggtgcgcggttgagcgtggagtcgtgggactgtgcatggaggtgtggggctccccgcacctgagcacccccgcataacaccccagtcccctctggaccctcttcaaggaagttcagttctttattgggctctccactacactgtgagtgccctcctcaggcgagagaacgttctggctcttctcttgccccttcagcccctgttaatcggacagagatggcagggctgtgtctccacggccggaggctctcatagtcagggcacccacagcggttccccacctgccttctgggcagaatacactgccacccataggtcagcatctccactcgtgggccatctgcttaggttgggttcctctggattctggggagattgggggttctgttttgatcagctgattcttctgggagcaagtgggtgctcgcgagctctccagcttcctaaaggtggagaagcacagacttcgggggcctggcctggatccctttccccattcctgtccctgtgcccctcgtctgggtgcgttagggctgacatacaaagcaccacagtgaaagaacagcagtatgcctcctcactagccaggtgtgggcgggtgggtttcttccaaggcctctctgtggccgtgggtagccacctctgtcctgcaccgctgcagtcttccctctgtgtgtgctcctggtagctctgcgcatgctcatcttcttataagaacaccatggcagctgggcgtagtggctcacgcctataatcccagcactttgggaggctgaggcaggcagatcacgaggtcaggagttcgagaccaacctgaccaacagggtgaaacctcgtctctactaaaaatacaaaaatacctgggcgtggtggtggtgcgcgcctataatcccagctactcaggaggctgaggcaggagaatcgcttgaacccaggaggcagaggttgcagtgagccgagatagtgccactgcactccagtttgagcaacagagcgagactctgtctcaaaacaaaataaaacaaaccaaaaaaacccaccatggcttagggcccagcctgatgacctcatttttcacttagtcacctctctaaaggccctgtctccaaatagagtcacattctaaggtacgggggtgttggggaggggggttagggcttcaacatgtgaatttgcggggaccacaattcagcccaggaccccgctcccgccacccagcactggggagctggggaagggtgaagaggaggctgggggtgagaaggaccacagctcactctgaggctgcagatgtgctgggccttctgggcactgggcctcggggagctagggggctttctggaaccctgggcctgcgtgtcagcttgcctcccccacgcaggcgctctccacaccattgaagttcttatcacttgggtctgagcctggggcatttggacggagggtggccaccagtgcacatgggcaccttgcctcaaaccctgccacctccccccacccaggatcccccctgcccccgaacaagcttgtgagtgcagtgtcacatcccatcgggatggaaatggacggtcgggttaaaagggacgcatgtgtagaccctgcctctgtgcatcaggcctcttttgagagtccctgcgtgccaggcggtgcacagaggtggagaagactcggctgtgccccagagcacctcctctcatcgaggaaaggacagacagtggctcccctgtggctgtggggacaagggcagagctccctggaacacaggagggagggaaggaagagaacatctcagaatctccctcctgatggcaaacgatccgggttaaattaaggtccggccttttcctgctcaggcatgtggagcttgtagtggaagaggctctctggaccctcatccaccacagtggcctggttagagaccttggggaaataactcacaggtgacccagggcctctgtcctgtaccgcagctgagggaaactgtcctgcgcttccactggggacaatgcgctccctcgtctccagactttccagtcctcattcggttctcgaaagtcgcctccagaagccccatcttgggaccaccgtgactttcattctccagggtgcctggccttggtgctgcccaagaccccagaggggccctcactggcctttcctgccttttctcccattgcccacccatgcacccccatcctgctccagcacccagactgccatccaggatctcctcaagtcacataacaagcagcacccacaaggtgctcccttccccctagcctgaatctgctgctccccgtctggggttccccgcccatgcacctctgggggcccctgggttctgccataccctgccctgtgtcccatggtggggaatgtccttctctccttatctcttcccttcccttaaatccaagttcagttgccatctcctccaggaagtcttcctggattcccctctctcttcttaaagcccctgtaaactctgaccacactgagcatgtgtctgctgctccctagtctgggccatgagtgagggtggaggccaagtctcatgcatttttgcagcccccacaagactgtgcaggtggccggccctcattgaatgcggggttaatttaactcagcctctgtgtgagtggatgattcaggttgccagagacagaaccctcagcttagcatgggaagtagcttccctgttgaccctgagttcatctgaggttggcttggaaggtgtgggcaccatttggcccagttcttacagctctgaagagagcagcaggaatggggctgagcagggaagacaactttccattgaaggcccctttcagggccagaactgtccctcccaccctgcagctgccctgcctctgcccatgaggggtgagagtcaggcgacctcatgccaagtgtagaaaggggcagacgggagccccaggttatgacgtcaccatgctgggtggaggcagcacgtccaaatctactaaagggttaaaggagaaagggtgacttgacttttcttgagatattttgggggacgaagtgtggaaaagtggcagaggacacagtcacagcctcccttaaatgccaggaaagcctagaaaaattgtctgaaactaaacctcagccataacaaagaccaacacatgaatctccaggaaaaaagaaaaagaaaaatgtcatacagggtccatgcacaagagcctttaaaatgacccgctgaagggtgtcaggcctcctcctcctggactggcctgaaggctccacgagcttttgctgagacctttgggtccctgtggcctcatgtagtacccagtatgcagtaagtgctcaataaatgtttggctacaaaagaggcaaagctggcggagtctgaagaatccctcaaccgtgccggaacagatgctaacaccaaagggaaaagagcaggagccaagtcacgtttgggaacctgcagaggctgaaaactgccgcagattgctgcaaatcattgggggaaaaacggaaaacgtctgttttcccctttgtgcttttctctgttttcttctttgtgcttttctctgttttcaggatttgctacagtgaacatagattgctttggggccccaaatggaattattttgaaaggaaaatgcagataatcaggtggccgcactggagcaccagctgggtaggggtagagattgcaggcaaggaggaggagctgggtggggtgccaggcaggaagagcccgtaggccccgccgatcttgtgggagtcgtgggtggcagtgttccctccagactgtaaaagggagcacctggcgggaagagggaattcttttaaacatcattccagtgcccgagcctcctggacctgttgtcatcttgaggtgggcctcccctgggtgactctagtgtgcagcctggctgagactcagtggccctgggttcttactgctgacacctaccctcaacctcaaccactgcggcctcctgtgcaccctgatccagtggctcattttccactttcagtcccagctctatccctatttgcagtttccaagtgcctggtcctcagtcagctcagacccagccaggccagcccctggttcccacatcccctttgccaagctcatccccgccctgtttggcctgcgggagtgggagtgtgtccagacacagagacaaaggaccagcttttaaaacattttgttggggccaggtgtggtggctcacacctaatcccaacacctggggaggccaaggcagaaggatcacttgagtccaggagttcaagaccagcctgggcaacatagggagaccctgtctctacaattttttttttaattagctgggcctgttggcactctcctgtagttccagctactctagaggctgaggtgggaggactgcttgagcctgggaggtcagggctgcaatgagccatgttcacaccactgaacgccagcctgggcgagaccctgtatcaaaaaagtaaagtaaaatgaatcctgtacgttatattaaggtgccccaaattgtacttagaaggatttcatagttttaaatacttttgttatttaaaaaattaaatgactgcagcatataaattaggttcttaatggaggggaaaaagagtacaagaaaagaaataagaatctagaaacaaagataagagcagaaataaaccagaaaacacaaccttgcactcctaacttaaaaaaaaaaatgaagaaaacacaaccagtaaaacaacatataacagcattaagagctggctcctggctgggcgcggtggcgcatgcctgtaatcccaacactttgggaggccgatgctggaggatcacttgagaccaggagttcaaggttgcagtgagctatgatcataccactacaccctagcctgggcaacacagtgagactgagactctattaaaaaaaaaatgctggttccttccttatttcattcctttattcattcattcagacaacatttatggggcacttctgagcaccaggctctgtgctaagagcttttgcccccagggtccaggccaggggacaggggcaggtgagcagagaaacagggccagtcacagcagcaggaggaatgtaggatggagagcttggccaggcaaggacatgcagggggagcagcctgcacaagtcagcaagccagagaagacaggcagacccttgtttgggacctgttcagtggcctttgaaaggacagcccccacccggagtgctgggtgcaggagctgaaggaggatagtggaacactgcaacgtggagctcttcagagcaaaagcaaaataaacaactggaggcagctggggcagcagagggtgtgtgttcagcactaaggggtgtgaagcttgagcgctaggagagttcacactggcagaagagaggttggggcagctgcaagcctctggacatcgcccgacaggacagagggtggtggacggtggccctgaagagaggctcagttcagctggcagtggccgtgggagtgctgaagcaggcaggctgtcggcatctgctggggacggttaagcaggggtgagggcccagcctcagcagcccttcttggggggtcgctgggaaacatagaggagaactgaagaagcagggagtcccagggtccatgcagggcgagagagaagttgctcatgtggggcccaggctgcaggatcaggagaactggggaccctgtgactgccagcggggagaagggggtgtgcaggatcatgcccagggaagggcccaggggcccaagcatgggggggcctggttggctctgagaagatggagctaaagtcactttctcggaggatgtccaggccaatagttgggatgtgaagacgtgaagcagcacagagcctggaagcccaggatggacagaaacctacctgagcagtggggctttgaaagccttggggcggggggtgcaatattcaagatggccacaagatggcaatagaatgctgtaactttcttggttctgggccgcagcctgggtggctgcttccttccctgtgtgtattgatttgtttctcttttttgagacagagtcttgctgggttgcccaggctggagtgcagtggtgcgatcatagctcactgcagccttgaagtcctgagctcaagagatccttccacctcagcctcctgagtagttgggaccacaggcttgcaccacagtgcccaactaatttcttatattttttgtagagatggggtttcactgtgtcgcccaggatggtcttgaactcctgggctcaagtgatcctcctgcctcagcctcgcaaattgctgggattacaggtgtgagccaccatgcccgaccttctctttttaagggcgtgtgtgtgtgtgtgtgtgtgtgggcgcactctcgtcttcaccttcccccagccttgctctgtctctacccagtcacctctgcccatctctccgatctgtttctctctccttttacccctctttcctccctcctcatacaccactgaccattatagagaactgagtattctaaaaatacattttatttatttattttgagacagagtctcactctgtcacccaggctggagtgcagtggtgcaatctcggctcactgcaacctccgcctcccaggttgaagcaactctcctgcctcagcctccctagtagctgggattacaagcacacaccaccatgcctagcaaatttttatatttttagtagaggaggagtgtcaccatgtttgccaagctggtctcaaactcctggcctcaggtgatctgcctaccttggtctcccaaagtgctgggattacaggtgtgagccaccacgcctgcccttaaaaatacattatatttaatagcaaagccccagttgtcactttaaaaagcatctatgtagaacatttatgtggaataaatacagtgaatttgtacgtggaatcgtttgcctctcctcaatcagggccagggatgcaggtgagcttgggctgagatgtcagaccccacagtaagtggggggcagagccaggctgggaccctcctctaggacagctctgtaactctgagaccctccaggcatcttttcctgtacctcagtgcttctgaaaaatctgtgtgaatcaaatcattttaaaggagcttgggttcatcactgtttaaaggacagtgtaaataattctgaaggtgactctaccctgttatttgatctcttctttggccagctgacttaacaggacatagacaggttttcctgtgtcagttcctaagctgatcaccttggacttgaagaggaggcttgtgtgggcatccagtgcccaccccgggttaaactcccagcagagtattgcactgggcttgctgagcctggtgaggcaaagcacagcacagcgagcaccaggcagtgctggagacaggccaagtctgggccagcctgggagccaactgtgaggcacggacggggctgtggggctgtggggctgcaggcttggggccagggagggagggctgggctctttggaacagccttgagagaactgaacccaaacaaaaccagatcaaggtctagtgagagcttagggctgctttgggtgctccaggaaattgattaaaccaagtggacacacacccccagccccacctcaccacagcctctccttcagggtcaaactctgaccacagacatttctcccctgactaggagttccctggatcaaaattgggagcttgcaacacatcgttctctcccttgatggtttttgtcagtgtctatccagagctgaagtgtaatatatatgttactgtagctgagaaattaaatttcaggattctgatttcataatgacaaccattcctcttttctctcccttctgtaaatctaagattctataaacggtgttgacttaatgtgacaattggcagtagttcaggtctgctttgtaaatacccttgtgtctattgtaaaatctcacaaaggcttgttgccttttttgtggggttagaacaagaaaaagccacatggaaaaaaaatttcttttttgtttttttgtttgcttgtttttttgagacagagtttcactctgtcgcccaggctggagtgcagtggtgcgatctccgcccactgcaagctccacctcccgggttcatgctattctcctgtctcagcctcccaagtagctgggactgcaggtgcccgccaccacacctggctaatttttttgtatttttagtagagacggggtttcaccgtgttagccaggatggtctcaatctcctgacctcgtcatctgcctgcctcggcctcccaaagtgctgagattacaggcgtgagccaccgtgcccggccagaaaaaaacatttctaagtatgtggcagatactgaattattgcttaatgtcctttgattcatttgtttaatttctttaatggattagtacagaaaacaaagttctcttccttgaaaaactggtaagttttctttgtcagataaggagagttaaataacccatgacatttccctttttgcctcggcttccaggaagctcaaagttaaatgtaatgatcactcttgtaattatcagtgttgatgcccttcccttcttctaatgttactctttacattttcctgctttattattgtgtgtgttttctaattctaagctgttcccactcctttctgaaagcaggcaaatcttctaagccttatccactgaaaagttatgaataaaaaatgatcgtcaagcctacaggtgctgaggctactccagaggctgaggccagaggaccacttgagcccaggaatttgagacctgggctgggcagcatagcaagactctatctccattaaaactatttttttttatttaaaaaataatccgcaaagaaggagtttatgtgggattccttaaaatcggagggtggcatgaattgattcaaagacttgtgcagagggcgacagtgactccttgagaagcagtgtgagaaagcctgtcccacctccttccgcagctccagcctgggctgaggcactgtcacagtgtctccttgctggcaggagagaatttcaacattcaccaaaaagtagtattgtttttattaggtttatgaggctgtagccttgaggacagcccaggacaactttgttgtcacatagatagcctgtggctacaaactctgagatctagattcttctgcggctgcttctgacctgagaaagttgcggaacctcagcgagcctcacatggcctccttgtccttaacgtggggacggtgggcaagaaaggtgatgtggcactagagatttatccatctctaaaggaggagtggattgtacattgaaacaccagagaaggaattacaaaggaagaatttgagtatctaaaaatgtaggtcaggcgctcctgtgttgattgcagggctattcacaatagccaagatttggaagcaacccaagtgtccatcaacagacaaatggataaagaaaatgtggtgcatatacacaatggaatactattcagccatgaaaaagaatgagaatctgtcatttgaaacaacatggatggaactggaggacattatgttaagtgaaataagccagacagaaggacagacttcacatgttctcacacatttgtgggagctaaaaattaaactcatggagatagagagtagaaggatggttaccagaggctgaggagggtggaggggagcagggagaaagtagggatggttaatgggtacaaaaacgtagttagcatgcatagatctagtattggatagcacagcagggtgacgacagccaacagtaatttatagtacatttaaaaacaactaaaagagtgtaactggactggctaacatggtgaaaccccgtctctactaaaaatacaaaaattagctgggcacggtggctcacgcctgtaatcccagcactttgggaggccgaggcgggccgatcacgaggtcaggagatcgagaccatcctagctaacatggtgaaaccccgtctctactacaaatacaaaaaaaagaaaaaattagccgggcatggtggtgggcgcctgtagtcccagctactcgggaggctgaggcaggagaatggcgtgaacccgggaggcggagcttgcagtgagccgagatcgcgccactgcactccagcctgggcgacaaggcaagattctatctcaaaaaaataaaaataaaataaaataaaataataaaataaaataaaataaaataaaataaaataaataaaataaaatgtataattggaatgtttataacacaagaaatgataaatgcttgaggtgatagataccccattcaccgtgatgtgattattgcacaatgtatgtctgtatctaaatatctcatgtaccccacaagtatatacacctactatgtacccatataaatttaaaattaaaaaattataaaacaaaaataaataagtaaattaaaatgtaggctggacaccgtggttcacgcctgtaatcccagtgctttgtgaggctgaggtgagagaatcacttgagcccaggagtttgagaccggcctgggtgacatagcgagaccccatcatcacaaagaatttttaaaaattagctgggcgtggtagcacataccggtagttccagctacttgggagaccgaggcaggaggattgcttgagcccaggagtttaaggctgcagtgagctacgatggcgccactgcattccagcctgggtgacagagtgagagcttgtctctattttaaaaataataaaaagaataaataaaaataaattaaaatgtaaatatgtgcatgttagaaaaaatacacccatcagcaaaaagggggtaaaggagcgatttcagtcataattggagagatgcagaataagccagcaatgcagtttcttttattttggtcaaaaaaaataagcaaaacaatgttgtaaacacccagtgctggcagcaatgtggtgaggctggctctctcaccagggctcacagggaaaactcatgcaacccttttagaaagccatgtggagagttgtaccgagaggttttagaatatttataactttgacccagaaattctattctaggactctgtgttatgaaaataacccatcatatggaaaaagctcctttcagaaagaggttcatgggaggctgtttgtattttttttttctttgcatcaaatccagctcctgcaggactgtttgtattattgaagtacaaagtggaatcaatacaaatgttggatagcaggggaacaatattcacaaaatggaatgggacatagtattaaacatagtgcttctgatgaccgtagaccatagacaatgcttaggatatgatatcacttcttttgttgttttttgtattttgagacgaagtctcattctgtcacccaggctggagttcagtggcgccatctcagctcactgcaacctccatctcccgggttcaagctattctccttcctcaacctcccgagtagctgggttgcgcaccaccatgcctggctaacttttgtatttttagtacagacggggtttcaccacgttggccaggctgctcttgaactcctgacgtcaggtgatccaccagccttgacctcccaaagtgctaggattacaggagccactgtacccagcctaggatatgatatcacttcttagagcaagatacaaaattgcatgtgcacaataattctaccaagtataggtatacaggggtagttatatataaatgagacttcaaggaaatacaacaaaatgcaatcgtgattgtgttagggtggtaagaaaacggtttttgctttgatgagctctgttttttaaaatcgttatattttctaataaaaatacatagtcttttgaaggaacataaaagattatgaagaaatgagttagatattgattcctattgaagattcagacaagtaaaattaaggggaaaaaaaacgggatgaaccagaagtcaggctggagttccaaccccagatccgacagcccaggctgatggggcctccagggcagtggtttccacccagcattctcaaaagagccactgaggtctcagtgccattttcaagatttcggaagcggcctgggcacggctggtccttcactgggatcaccacttggcaattatttacacctgagacgaatgaaaaccagagtgctgagattacaggcatggtggcttacgcttgtaatcggctttgggaagccgaggtgggctgattgcttgagcccaggagtttcaaactatcctggacaacatagcatgacctcgtctctacaaaaaatacaaaaaatttgccaggtgtggtggcatgtgcctgtggtcccagctacttgggaggctgaagtaggagaatcccctgagccctgggaagtcgaggctgcactgagccgtgatggtgtcactgcactccagcctgggtgacaaagtgagaccctatctcacaaagaaaaaaaacaaaacaaaaaacccaaagcacactgtttccactgtttccagagttcctgagaggaaaggtcaccgggtgaggaagacgttctcactgatctggcagagaaaatgtccagtttttccaactccctaaaccatggttttctatttcatagttcttaggcaaattggtaaaaatcatttctcatcaaaacgctgatattttcacacctccctggtgtctgcagaaagaaccttccagaaatgcagtcgtgggagacccatccaggccacccctgcttatggaagagctgagaaaaagccccacgggagcatttgctcagcttccgttacgcacctagtggcattgtgggtgggagagggctggtgggtggatggaaggagaaggcacagcccccccttgcagggacagagccctcgtacagaagggacaccccacatttgtcttccccacaaagcggcctgtgtcctgcctacggggtcagggcttctcaaacctggctgtgtgtcagaatcaccaggggaacttttcaaaactagagagactgaagccagactcctagattctaattctaggtcagggctaggggctgagattgtaaaaatccacaggtgattctgatgcccggcaggcttgagaacagccgcagggagttctctgggaatgtgccggtgggtctagccaggtgtgagtggagatgccggggaacttcctattactcactcgtcagtgtggccgaacacatttttcacttgacctcaggctggtgaacgctcccctctggggttcaggcctcacgatgccatccttttgtgaagtgaggacctgcaatcccagcttcgtaaagcccgctggaaatcactcacacttctgggatgccttcagagcagccctctatcccttcagctcccctgggatgtgactcgacctcccgtcactccccagactgcctctgccaagtccgaaagtggaggcatccttgcgagcaagtaggcgggtccagggtggcgcatgtcactcatcgaaagtggaggcgtccttgcgagcaagcaggcgggtccagggtggcgtgtcactcatccttttttctggctaccaaaggtgcagataattaataagaagctggatcttagcaacgtccagtccaagtgtggctcaaaggataatatcaaacacgtcccgggaggcggcagtgtgagtaccttcacacgtcccatgcgccgtgctgtggcttgaattattaggaagtggtgtgagtgcgtacacttgcgagacactgcatagaataaatccttcttgggctctcaggatctggctgcgacctctgggtgaatgtagcccggctccccacattcccccacacggtccactgttcccagaagccccttcctcatattctaggagggggtgtcccagcatttctgggtcccccagcctgcgcaggctgtgtggacagaatagggcagatgacggaccctctctccggaccctgcctgggaagctgagaatacccatcaaagtctccttccactcatgcccagccctgtccccaggagccccatagcccattggaagttgggctgaaggtggtggcacctgagactgggctgccgcctcctcccccgacacctgggcaggttgacgttgagtggctccactgtggacaggtgacccgtttgttctgatgagcggacaccaaggtcttactgtcctgctcagctgctgctcctacacgttcaaggcaggagccgattcctaagcctccagcttatgcttagcctgcgccaccctctggcagagactccagatgcaaagagccaaaccaaagtgcgacaggtccctctgcccagcgttgaggtgtggcagagaaatgctgcttttggcccttttagatttggctgcctcttgccaggagtggtggctcgtgcctgtaattccagcactttgggagactaaggcgggaggttcgcttgagcccaggagttcaagaccagcctgggcaacaatgagacccctgtgtctacaaaaagaattaaaattagccaggtgtggtggcacgcacctgtagtcccagctacttgggaggctgaggtgggaggattgcctgagtccgggaggcggaagttgcaaggagccatgatcgcgccactgcacttcaacctaggcaacagagtgagactttgtctcaaaaaacaatcatataataattttaaaataaatagatttggcttcctctaaatgtccccggggactccgtgcatcttctgtggagtgtctccgtgagattcgggactcagatcctcaagtgcaactgacccacccgataagctgaggcttcatcatcccctggccggtctatgtcgactgggcacccgaggctcctctcccaccagctctcttggtcagctgaaagcaaactgttaacaccctggggagctggacgtatgagacccttggggtgggaggcgttgatttttgagagcaatcacctggccctggctggcagtaccgggacactgctgtggctccggggtgggctgtctccagaaaatgcctggcctgaggcagccacccgcatccagcccagagggtttattcttgcaatgtgctgctgcttcctgccctgagcacctggatcccggcttctgccctgaggccccttgagtcccacaggtagcaagcgcttgccctgcggctgctgcatggggctaactaacgcttcctcaccagtgtctgctaagtgtctcctctgtctcccacgccctgctctcctgtccccccagtttgtctgctgtgaggggacagaagaggtgtgtgccgcccccacccctgcccgggcccttgttcctgggattgctgttttcagctgtttgagctttgatcctggttctctggcttcctcaaagtgagctcggccagaggaggaaggccatgtgctttctggttgaagtcaagtctggtgccctggtggaggctgtgctgctgaggcggagctggggagagagtgcacacgggctgcgtggccaacccctctgggtagctgatgcccaaagacgctgcagtgcccaggacatctgggacctccctggggcccgcccgtgtgtcccgcgctgtgttcatctgcgggctagcctgtgacccgcgctgtgctcgtctgcgggctagcctgtgtcccgcgctctgcttgtctgcggtctagcctgtgacctggcagagagccaccagatgtcccgggctgagcactgccctctgagcaccttcacaggaagcccttctcctggtgagaagagatgccagcccctggcatctgggggcactggatccctggcctgagccctagcctctccccagcctgggggccccttcccagcaggctggccctgctccttctctacctgggacccttctgcctcctggctggaccctggaagctctgcagggcctgctgtccccctccctgccctccaggtatcctgaccaccggccctggctcccactgccatccactcctctcctttctggccgttccctggtccctgtcccagcccccctccccctctcacgagttacctcacccaggccagagggaagagggaaggaggccctggtcataccagcacgtcctcccacctccctcggccctggtccaccccctcagtgctggcctcagagcacagctctctccaagccaggccgcgcgccatccatcctccctgtcccccaacgtccttgccacagatcatgtccgccctgacacacatgggtctcagccatctctgccccagttaactccccatccataaagagcacatgccagccgacaccaaaataattcgggatggttccagtttagacctaagtggaaggagaaaccaccacctgccctgcaccttgttttttggtgaccttgataaaccatcttcagccatgaagccagctgtctcccaggaagctccagggcggtgcttcctcgggagctgactgataggtgggaggtggctgcccccttgcaccctcaggtgaccccacacaaggccactgctggaggccctggggactccaggaatgtcaatcagtgacctgccccccaggccccacacagccatggctgcatagaggcctgcctccaagggacctgtctgtctgccactgtggagtccctacagcgtgccccccacaggggagctggttctttgactgagatcagctggcagctcagggtcatcattcccagagggagcggtgccctggaggccacaggcctcctcatgtgtgtctgcgtccgctcgagcttactgagacactaaatctgttggtttctgctgtgccacctacccaccctgttggtgttgctttgttcctattgctaaagacaggaatgtccaggacactgagtgtgcaggtgcctgctggttctcacgtccgagctgctgaactccgctgggtcctgcttactgatggtctttgctctagtgctttccagggtccgtggaagcttttcctggaataaagcccacgcatcgaccctcacagcgcctcccctctttgaggcccagcagataccccactcctgcctttccagcaagatttttcagatgctgtgcatactcatcatattgatcacttttttcttcatgcctgattgtgatctgtcaatttcatgtcaggaaagggagtgacatttttacacttaagcgtttgctgagcaaatgtctgggtcttgcacaatgacaatgggtccctgtttttcccagaggctcttttgttctgcagggattgaagacactccagtcccacagtccccagctcccctggggcagggttggcagaatttcgacaacacatttttccaccctgactaggatgtgctcctcatggcagctgggaaccactgtccaataagggcctgggcttacacagctgcttctcattgagttacacccttaataaaataatcccattttatcctttttgtctctctgtcttcctctctctctgcctttcctcttctctctcctcctctctcatctccag

SEQ ID NO: 79 - DNA, 18 bp, Artificial
  Synthetic oligonucleotide
tatctgcacctttggtag

SEQ ID NO: 80 - DNA, 21 bp, Artificial
  Synthetic oligonucleotide
tgaaggtactcacactgccgc

(19) United States
(12) Patent Application Publication
(10) Pub. No.: US 2010/0196335 A1
(43) Pub. Date: Aug. 5, 2010

(54) Methods and Compositions for Regulated Expressions of Nucleic Acid at Post-Transcriptional Level

(76) Inventors: Richard J. Samulski, Chapel Hill, NC (US); Kyson Xiaohuai Chou, Irvine, CA (US)

Correspondence Address: MYERS BIGEL SIBLEY & SAJOVEC, PO BOX 37428, RALEIGH, NC, 27627, US

(21) Appl. No.: 11/919,267

(22) PCT Filed: Apr. 28, 2006
(86) PCT No.: PCT/US06/16514

§ 371 Date: Sep. 21, 2009

Related U.S. Application Data
(60) Provisional application No. 60/676,139, filed on Apr. 29, 2005.

(51) Int. Cl. A61K 35/12; C07H 21/02; C12N 15/74; C12N 5/10; A61K 31/7088; C12P 21/00; C12P 19/34; A61K 31/7105; C12Q 1/68; A61P 43/00

(52) U.S. Cl. 424/93.21; 536/23.1; 435/320.1; 435/325; 514/44 R; 435/68.1; 435/91.5; 435/366; 514/44 A; 435/6

(57) ABSTRACT

The present invention provides an isolated nucleic acid comprising: a) at least one first nucleotide sequence encoding a heterologous nucleotide sequence of interest; and b) at least two second heterologous nucleotide sequences, wherein each second heterologous nucleotide sequences comprises: i) a first set of splice elements defining a first intron that is removed by splicing to produce a first RNA molecule that imparts a biological function in the absence of activity at a second set of splice elements; and ii) the second set of splice elements defining one or more introns different from said first intron, wherein said one or more introns different from said first intron are removed by splicing to produce no RNA molecule and/or a second RNA molecule that docs not impart a biological function, when said second set of splice elements is active. Further provided are methods of using the nucleic acid of this invention to regulate transgene expression.

### RELATED APPLICATIONS

This application claims the benefit, under 35 U.S.C.§119(e), of U.S. Provisional Application No. 60/676,139, filed Apr. 29, 2005, the entire contents of which are incorporated by reference herein.

### FIELD OF THE INVENTION

The present invention relates to compositions and methods of their use for regulating nucleic acid expression at the post-transcriptional level.

### BACKGROUND OF THE INVENTION

Recent developments in gene therapy have raised hopes for effective treatment via such protocols of a variety of long-term diseases. However, it has become clear that control of gene expression is desirable for safe and flexible treatment. Many different regulation systems have been tested in gene therapy vectors and have been demonstrated to regulate gene expression both in vitro and in vivo, including the tetracycline responsive system, rapamycin regulated protein dimerization and many others. The majority of these systems function by controlling transcriptional activation and are derived from endogenous mammalian gene regulation pathways or artificial hybrids of drug responsive components combined with transcription activation domains. These systems require expression of one or more proteins in addition to the transgene and administration of an exogenous drug or other compound to activate or repress transcription. For gene therapy vectors with restricted packaging capacity such as adeno-associated virus (AAV) vectors or retroviral vectors, the inclusion of additional genes can limit transgene size or require the use of two separate vectors to deliver all necessary components. While these systems can be used to effectively control transcription, there are many cases where these large systems are impractical or unwieldy.

Endogenous gene expression is regulated at several post-transcriptional levels that could also be exploited for control of exogenous gene expression. RNA production is controlled by the rate of transcription, but functional RNA requires correct splicing before the correct gene product can be produced. By regulating splicing of the transgene RNA, production of the gene product can be controlled.

The immune response to gene therapy vectors has also been an important consideration, especially for diseases that require lengthy treatment. The immune system can respond not only to the vectors themselves, but also to proteins they produce. Because many of the most successful regulation systems involve hybrid or foreign proteins, these are particularly susceptible to inducing immune reactions and several systems have been shown to induce such immune reactions in rodent and non-human primates.

The present invention overcomes previous shortcomings in the art by providing compositions and methods for controlled expression of genes without the disadvantages of previously described gene expression systems.

### SUMMARY OF THE INVENTION

The present invention provides an isolated nucleic acid comprising: A) at least one first nucleotide sequence encoding a heterologous nucleotide sequence of interest; and B) at least two heterologous second nucleotide sequences, wherein each heterologous second nucleotide sequence comprises: i) a first set of splice elements defining a first intron that is removed by splicing to produce a first RNA molecule that imparts a biological function in the absence of activity at a second set of splice elements; and ii) the second set of splice elements defining one or more introns different from said first intron, wherein said one or more introns different from said first intron are removed by splicing to produce no RNA molecule and/or a second RNA molecule that does not impart a biological function, when said second set of splice elements is active, wherein the heterologous second nucleotide sequences are selected from the group consisting of: a) second nucleotide sequences in tandem within said first nucleotide sequence, b) second nucleotide sequences spaced at least 25 base pairs apart within said first nucleotide sequence, c) second nucleotide sequences spaced at least 50 base pairs apart within said first nucleotide sequence, d) second nucleotide sequences spaced at least 75 base pairs apart within said first nucleotide sequence, e) second nucleotide sequences spaced at least 100 base pairs apart within said first nucleotide sequence, f) second nucleotide sequences spaced at least 200 base pairs apart within said first nucleotide sequence, g) second nucleotide sequences spaced at least 300 base pairs apart within said first nucleotide sequence, h) second nucleotide sequences wherein a primary second nucleotide sequence is located between a promoter and said first nucleotide sequence and a secondary second nucleotide sequence is located within said first nucleotide sequence; and i) second nucleotide sequences wherein a primary second nucleotide sequence is located between an open reading frame and a poly(A) tail or poly A signal in said first nucleotide sequence and a secondary second nucleotide sequence located within said open reading frame of said first nucleotide sequence.

Further provided herein is an isolated nucleic acid comprising: A) at least one first nucleotide sequence encoding a heterologous nucleotide sequence of interest and B) at least one second heterologous nucleotide sequence, comprising: i) a first set of splice elements defining a first intron that is removed by splicing to produce a first RNA molecule that imparts a biological function in the absence of activity at a second set of splice elements; and ii) the second set of splice elements defining a intron different from said first intron, wherein said second intron is removed by splicing to produce no RNA molecule and/or a second RNA molecule that does not impart a biological function, when said second set of splice elements is active, wherein the second nucleotide sequence is selected from the group consisting of: a) SEQ ID NO:50 (IVS2-654 intron with 564CT mutation), b) SEQ ID NO:51 (IVS2-654 intron with 657G mutation), c) SEQ ID NO:52 (IVS2-654 intron with 658T mutation), d) SEQ ID NO:20 (IVS2-654 intron with 657GT mutation), e) SEQ ID NO:53 (IVS2-654 intron with 200 by deletion), f) SEQ ID NO:68 (IVS2-654 intron with only 197 bp), g) SEQ ID NO:55 (IVS2-654 intron with 6A mutation), h) SEQ ID NO:56 (IVS2-654 intron with 564C mutation), i) SEQ ID NO:57 (IVS2-654 intron with 841A mutation), j) SEQ ID NO:59 (IVS2-705 intron with 564CT mutation), SEQ ID NO:50 (IVS2-654 intron with 564CT mutation), SEQ ID NO:54 (IVS2-654 intron with 425 by deletion), SEQ ID NO:69 (IVS2-654 intron with only 247 bp), SEQ ID NO:59 (IVS2-705 intron with 564CT mutation), SEQ ID NO:60 (IVS2-705 intron with 657G mutation), SEQ ID NO:61 (IVS2-705 intron with 658T mutation), SEQ ID NO:62 (IVS2-705 intron with 657GT mutation), SEQ ID NO:63 (IVS2-705 intron with 200 by deletion), SEQ ID NO:64 (IVS2-705 intron with 425 by deletion) SEQ ID NO:65 (IVS2-705 intron with 6A mutation), SEQ ID NO:66 (IVS2-705 intron with 564C mutation), SEQ ID NO:67 (IVS2-705 intron with 841A mutation) and any combination thereof.

Additionally provided herein is a method for producing a protein, comprising; a) contacting a blocking oligonucleotide with the nucleic acid of this invention under conditions that permit splicing, wherein the blocking oligonucleotide blocks a member of the second set of splice elements, resulting in removal of the first intron by splicing and production of the first RNA; and b) translating the first RNA to produce the protein.

Also provided herein is a method for producing an RNA that imparts a biological function, comprising: a) contacting a blocking oligonucleotide with the nucleic acid of this invention under conditions that permit splicing, wherein the blocking oligonucleotide blocks a member of the second set of splice elements, resulting in removal of the first intron by splicing and production of the first RNA; and b) translating the first RNA to produce the RNA that imparts biological function.

Furthermore, the present invention provides a method for producing an RNA that imparts a biological function, comprising: a) contacting a small molecule with the nucleic acid of this invention under conditions which permit splicing, wherein the small molecule blocks a member of the second set of splice elements, resulting in removal of the first intron and production of the first RNA; and b) translating the first RNA to produce the RNA that imparts a biological function.

Additionally provided herein is a method of regulating production of a heterologous RNA that imparts a biological function in a subject, comprising: a) introducing into the subject the nucleic acid of this invention; and b) introducing into the subject a blocking oligonucleotide and/or small molecule that blocks a member of the second set of splice elements, at a time when production of the heterologous RNA is desired, thereby regulating production of the heterologous RNA in the subject.

In further embodiments, the present invention provides a method of regulating production of a heterologous protein in a subject, comprising: a) introducing into the subject the nucleic acid of this invention; and b) introducing into the subject a blocking oligonucleotide and/or small molecule that blocks a member of the second set of splice elements, at a time when production of the heterologous protein is desired, thereby regulating production of the heterologous protein in the subject.

The present invention further provides a method of identifying a compound that blocks a member of the second set of splice elements of the nucleic acid of this invention, comprising: a) contacting the nucleic acid of this invention with the compound under conditions that permit splicing; and b) detecting the production of the first RNA of this invention and/or the production of the second RNA of this invention, whereby the production of the first RNA identifies a compound that blocks a member of the second set of splice elements of the nucleic acid of this invention.

Also provided herein is a method for inhibiting production of a heterologous RNA that imparts a biological function, comprising: a) contacting a small molecule with the nucleic acid of this invention under conditions which permit splicing, wherein the small molecule blocks a member of the first set of splice elements, resulting in removal of the second intron, thereby inhibiting production of the first RNA.

In addition, the present invention provides a method for inhibiting production of a heterologous protein, comprising: a) contacting a small molecule with the nucleic acid of this invention under conditions which permit splicing, wherein the small molecule blocks a member of the first set of splice elements, resulting in removal of the second intron, thereby inhibiting production of the first RNA.

In further embodiments, the present invention provides a method for inhibiting production of a heterologous RNA that imparts a biological function, comprising: a) contacting a blocking oligonucleotide with the nucleic acid of this invention under conditions which permit splicing, wherein the blocking oligonucleotide blocks a member of the first set of splice elements, resulting in removal of the second intron, thereby inhibiting production of the first RNA.

The present invention additionally provides a method of inhibiting production of a heterologous protein, comprising: a) contacting a blocking oligonucleotide with the nucleic acid of this invention under conditions which permit splicing, wherein the blocking oligonucleotide blocks a member of the first set of splice elements, resulting in removal of the second intron, thereby inhibiting production of the first RNA.

The foregoing and other objects and aspects of the present invention are explained in detail in the specification set forth below.

### BRIEF DESCRIPTION OF THE DRAWINGS

FIG. 1 is a schematic of a portion of a nucleic acid construct of this invention, showing the mechanism of regulating expression of the luciferase sequence, based on the presence or absence of an exogenous oligonucleotide, as described herein.

FIGS. 2A-B show AAV Luc expression in vivo after portal vein injection of 1×10¹¹ vector particles. One year and seven days after vector injection, 25 mg/kg of LNA oligonucleotide was administered (A ii; B at arrows) via intraperitoneal injection. Luciferase transgene activity was measured using real time imaging (A) and expressed as light unit X10⁶ over time. B: oligo=diamonds; no oligo=circles.

FIG. 3 shows AAT expression in vivo after oligonucleotide treatment. Mouse livers transduced with an AAV vector expressing an intron-regulated AAT coding sequence cassette were treated with 0.625 mg/200 μl of an LNA oligonucleotide for two days by intraperitoneal injection (arrow). Circulating levels of human AAT were analyzed by an ELISA assay of blood samples over time.

FIG. 4 shows the change in luciferase expression based on adding different mutations to the 654 mutant. A QuickChange™ Site-Directed Mutagenesis Kit (Stratagene) was used according to instructions to generate the following mutations (numbering is based on the number of base pairs away from the 5′ splice site of IVS-654): 6 T to A, 564 A to C, 564 AA to CT, 657 TA to GT and 841 C to A. The new intron was cloned into luciferase cDNA. 293 cells were transfected with vector and oligo as described herein.

### DETAILED DESCRIPTION OF THE INVENTION

As used herein, “a,” “an” or “the” can be singular or plural, depending on the context of such use. For example, “a cell” can mean a single cell or it can mean a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a composition of this invention, dose, time, temperature, and the like, is meant to encompass variations of ±20%, ±10%, ±5%, ±1%, ±0.5%, or even ±0.1% of the specified amount.

The present invention is based on the unexpected discovery that expression of a nucleic acid, such as an exogenous nucleic acid, can be regulated, e.g., in vivo, at the post-transcriptional level. Such regulation is based on the selective splicing of different introns associated with the nucleic acid, according to the presence or absence of an oligonucleotide, small molecule and/or other compound that selectively blocks splicing activity at specific sites. Thus, in one embodiment, the present invention provides an isolated nucleic acid comprising, consisting essentially of and/or consisting of: a) at least one (e.g., one, two, three, four or more) first exogenous nucleotide sequence encoding a heterologous nucleotide sequence of interest; and b) at least one (e.g., two, three, four or more) exogenous or heterologous second nucleotide sequences, wherein each second exogenous or heterologous second nucleotide sequence comprises: i) a first set of splice elements defining a first intron that is removed by splicing to produce a first RNA molecule that imparts a biological function in the absence of activity at a second set of splice elements; and ii) a second set of splice elements defining one or more introns different from said first intron, wherein said one or more introns different from said first intron are removed by splicing to produce no RNA molecule and/or a second RNA molecule that does not impart a biological function, when said second set of splice elements is active.

Numerous systems available, for example, from known mutated intron systems can be employed to make the compositions of this invention and to carry out the methods of this invention. For example, the β-globin mutated intron that causes certain thallesemias can be employed (e.g., SEQ ID NO:58; SEQ ID NO:18; SEQ ID NO:19, with and/or without additional mutations as described herein), (see, e.g., Suwanmanee et al. “Restoration of human beta-globin gene expression in murine and human IVS2-654 thalassemic erythroid cells by free uptake of antisense oligonucleotides” Mol. Pharmacol. (2002) 62:545-553, incorporated by reference herein in its entirety). Other systems include the mutant intron of the cystic fibrosis transmembrane conductance regulator (CFTR) gene (e.g., SEQ ID NO:70; SEQ ID NO:71 with and without additional mutations), (see e.g., Accession No. NC_{—}000007, nucleotides 116907253 to 117095951 from build 36 version 1 of NCBI genome annotation; Highsmith et al. (1994) “A novel mutation in the cystic fibrosis gene in patients with pulmonary disease but normal sweat chloride concentrations” New England Journal of Medicine 331:974-980, incorporated by reference herein in its entirety).

An additional system includes mutations in the dystrophin gene (SEQ ID NO:74; SEQ IDS NO:75 with and without additional mutations); (see, e.g., Accession No. NC_{—}000023, nucleotides 31047266 to 33267647 from build 36 version 1 of NCBI genome annotation; Tuffery-Giraud et al. (1999) “Point mutations in the dystrophin gene: evidence for frequent use of cryptic splice sites as a result of splicing defects” Human Mutation 14:359-368; Aartsma-Rus et al. (2004) “Antisense-induced multiexon skipping for Duchenne Muscular Dystrophy makes more sense” American Journal of Human Genetics 74:83-92; Chamberlain et al. (1991) “PCR analysis of dystrophin gene mutation and expression” J. Cell. Biochem. 46:255-259; Mann et al. (2001) “Antisense-induced exon skipping and synthesis of dystrophin in the mdx mouse” Proc. Natl. Acad. Sci. USA 98:42-47; Lu et al. (2003) “Functional amounts of dystrophin produced by skipping the mutated exon in the mdx dystrophic mouse” Nat. Med. 9:1009-1014; Kole et al. (2004) “RNA modulation, repair and remodeling by splice switching oligonucleotides” Acta Biochimica Polonica 51:373-378; all of the above being incorporated by reference herein in their entireties).

Yet another system that can be employed in the methods and compositions of this invention is the mutated tau gene that causes alternative splicing defects (e.g., SEQ ID NO:78); (see, e.g., Kalbfuss et al. “Correction of alternative splicing in tau in frontotemporal dementia and Parkinsonism linked to chromosome 17” J. Biol. Chem. 276:42986-42993 (2001); incorporated by reference herein in its entirety), as well as any other such mutated genes that produces a splicing defect, as are now known or later identified. Modified introns that introduce alternative splice sets can also be produced and tested according to methods well know to the ordinary artisan.

In a particular embodiment, the present invention provides an isolated nucleic acid comprising: A) at least one first nucleotide sequence encoding a heterologous nucleotide sequence of interest; and B) at least two heterologous second nucleotide sequences, wherein each heterologous second nucleotide sequence comprises: i) a first set of splice elements defining a first intron that is removed by splicing to produce a first RNA molecule that imparts a biological function in the absence of activity at a second set of splice elements; and ii) the second set of splice elements defining one or more introns different from said first intron, wherein said one or more introns different from said first intron are removed by splicing to produce no RNA molecule and/or a second RNA molecule that does not impart a biological function, when said second set of splice elements is active, wherein the heterologous second nucleotide sequences are selected from the group consisting of: a) second nucleotide sequences in tandem within said first nucleotide sequence, b) second nucleotide sequences spaced at least 25 base pairs apart within said first nucleotide sequence, c) second nucleotide sequences spaced at least 50 base pairs apart within said first nucleotide sequence, d) second nucleotide sequences spaced at least 75 base pairs apart within said first nucleotide sequence, e) second nucleotide sequences spaced at least 100 base pairs apart within said first nucleotide sequence, 1) second nucleotide sequences spaced at least 200 base pairs apart within said first nucleotide sequence, g) second nucleotide sequences spaced at least 300 base pairs apart within said first nucleotide sequence, h) second nucleotide sequences wherein a primary second nucleotide sequence is located between a promoter and said first nucleotide sequence and a secondary second nucleotide sequence is located within said first nucleotide sequence; and i) second nucleotide sequences wherein a primary second nucleotide sequence is located between an open reading frame and a poly(A) tail or poly A signal in said first nucleotide sequence and a secondary second nucleotide sequence located within said open reading frame of said first nucleotide sequence. Although these are specific examples of distances between introns, it is understood that the two or more introns can have any number of base pairs separating them, such as 2, 3, 4, 5, 6, 7, 8, 9, 10, 15, 20, 25, 30, 35, 40, 45, 50, 60, 70, 80, 90, 100, 200, etc. as described herein. It is further understood that the second nucleotide sequence of this invention can comprise one or more mutations in any combination, as described herein.

In further embodiments, the present invention provides an isolated nucleic acid comprising: A) at least one (e.g., one, two, three, four or more) first nucleotide sequence encoding a heterologous nucleotide sequence of interest and B) a second heterologous nucleotide sequence, comprising: i) a first set of splice elements defining a first intron that is removed by splicing to produce a first RNA molecule that imparts a biological function in the absence of activity at a second set of splice elements; and ii) the second set of splice elements defining at least one (e.g., one, two, three, four or more) intron different from said first intron, wherein said at least one intron different from said first intron is removed by splicing to produce no RNA molecule and/or a second RNA molecule that does not impart a biological function, when said second set of splice elements is active, wherein the second nucleotide sequence is selected from the group consisting of: a) SEQ ID NO:50 (IVS2-654 intron with 564CT mutation), b) SEQ ID NO:51 (IVS2-654 intron with 657G mutation), c) SEQ ID NO:52 (IVS2-654 intron with 658T mutation), d) SEQ ID NO:20 (IVS2-654 intron with 657GT mutation), e) SEQ ID NO:53 (IVS2-654 intron with 200 by deletion), f) SEQ ID NO:68 (IVS2-654 intron with only 197 bp), g) SEQ ID NO:55 (IVS2-654 intron with 6A mutation), h) SEQ ID NO:56 (IVS2-654 intron with 564C mutation), i) SEQ ID NO:57 (IVS2-654 intron with 841A mutation), j) SEQ ID NO:59 (IVS2-705 intron with 564CT mutation), k) SEQ ID NO:60 (IVS2-705 intron with 657G mutation), 1) SEQ ID NO:61 (IVS2-705 intron with 658T mutation), m) SEQ ID NO:62 (IVS2-705 intron with 657GT mutation), n) SEQ ID NO:63 (IVS2-705 intron with 200 by deletion), o) SEQ ID NO:64 (IVS2-705 intron with 425 by deletion), p) SEQ ID NO:65 (IVS2-705 intron with 6A mutation), q) SEQ ID NO:66 (IVS2-705 intron with 564C mutation), r) SEQ ID NO:67 (IVS2-705 intron with 841A mutation) and any combination thereof, including singly.

The first nucleotide sequence can encode, for example, is a protein or peptide, a nucleotide sequence having enzymatic activity as an RNA (e.g., RNAi), a nucleotide sequence encoding a ribozyme, a nucleotide sequence encoding an antisense sequence and/or a small nuclear RNA (snRNA), in any combination. Furthermore, the first nucleotide sequence can comprise one or more mutations and in some embodiments such mutations can play a role in defining splice sites and/or modulating splicing activity.

It is also understood that the first nucleotide sequences and the second nucleotide sequences of this invention can be the same and/or different in any combination of repeats and/or alternates in the isolated nucleic acid of this invention.

The second nucleotide sequence of this invention can be a nucleotide sequence that is a nucleotide sequence that defines an intron that comprises one or more mutations, the presence of which results in a first set of splice elements and a second set of splice elements. In some embodiments, the second nucleotide sequence can be a sequence that defines an intron-exon-intron region, wherein a mutation in either the intron and/or exon region results in the presence of a first set of splice elements and a second set of splice elements. In this latter embodiment, when the second set of splice elements is active, the result is production of an RNA comprising the exon of the intron-exon-intron region.

Further provided herein is a vector comprising a nucleic acid of this invention and a cell comprising the nucleic acid or vector of this invention. In some embodiments, he vector can be, but is not limited to a nonviral vector, a viral vector and a synthetic biological nanoparticle. Nonlimiting examples of a viral vector of this invention include an AAV vector, an adenovirus vector, a lentivirus vector, a retrovirus vector, a herpesvirus vector, an alphavirus vector, a poxvirus vector a baculovirus vector and a chimeric virus vector.

The present invention also provides various methods employing the nucleic acids of this invention. Thus, in some embodiments, the present invention provides a method for producing a protein and/or an RNA that imparts a biological function, comprising; a) contacting a blocking oligonucleotide with the nucleic acid of this invention under conditions that permit splicing, wherein the blocking oligonucleotide blocks a member of the second set of splice elements, resulting in removal of the first intron by splicing and production of the first RNA; and b) translating the first RNA to produce the protein and or to produce the RNA that imparts a biological function.

The blocking oligonucleotide and/or small molecule and/or other blocking compound of this invention can be introduced into a cell comprising the nucleic acid of this invention and such a cell can be in vitro or in a subject of this invention as described herein (e.g., an animal, which can be a human).

In additional embodiments, the present invention provides a method for producing a protein and or an RNA that imparts a biological function, comprising: a) contacting a small molecule with the nucleic acid of any of this invention under conditions which permit splicing, wherein the small molecule blocks a member of the second set of splice elements, resulting in removal of the first intron and production of the first RNA; and b) translating the first RNA to produce the protein and/or to produce the RNA that imparts a biological function.

In addition, the present invention provides a method of regulating production of a heterologous protein and/or RNA that imparts a biological function in a subject, comprising: a) introducing into the subject the nucleic acid of this invention; and b) introducing into the subject a blocking oligonucleotide and/or small molecule that blocks a member of the second set of splice elements, at a time when production of the heterologous protein and/or RNA is desired, thereby regulating production of the RNA in the subject.

Screening methods are also provided herein, such as a method of identifying a compound that blocks a member of the second set of splice elements of the nucleic acid of this invention, comprising: a) contacting the nucleic acid of this invention with the compound under conditions that permit splicing; and b) detecting the production of the first RNA and/or the production of the second RNA, whereby the production of the first RNA identifies a compound that blocks a member of the second set of splice elements.

In certain embodiments described herein, the transgene expression system is introduced (e.g., into a subject) in the OFF position and contact with a blocking oligonucleotide and/or small molecule of this invention switches the system to the ON position. Further provided herein are methods of turning a system which is introduced (e.g., into a subject) in the ON position to the OFF position, such as a method for inhibiting production of a heterologous protein and/or RNA that imparts a biological function, comprising: a) contacting a blocking oligonucleotide and/or a small molecule with the nucleic acid of this invention under conditions which permit splicing, wherein the small molecule blocks a member of the first set of splice elements, resulting in removal of the second intron, thereby inhibiting production of the first RNA.

An intron is a portion of eukaryotic DNA or RNA that intervenes between the coding portions, or “exons,” of that DNA or RNA. Introns and exons are transcribed from DNA into RNA termed “primary transcript, precursor to RNA” (or “pre-mRNA”). Introns must be removed from the pre-mRNA so that the protein encoded by the exons can be produced (the term “protein” as used herein refers to naturally occurring, wild type, or functional protein). The removal of introns from pre-mRNA and subsequent joining of the exons is carried out in the splicing process.

The splicing process is a series of reactions that are carried out on RNA after transcription (i.e., post-transcriptionally) but before translation and that are mediated by splicing factors. Thus, a “pre-mRNA” is an RNA that contains both exons and one or more introns, and a “messenger RNA (mRNA or RNA)” is an RNA from which any introns have been removed and wherein the exons are joined together sequentially so that the gene product can be produced therefrom, either by translation with ribosomes into a functional protein or by translation into a functional RNA.

The term “translation” as used herein includes the production of an amino acid chain (e.g., a peptide or polypeptide) directed by ribosomes that move along a messenger RNA comprising codons that encode the amino acid sequence. The term translation as used herein also includes the production of a functional RNA molecule (e.g., a ribozyme, antisense RNA, RNAi, snRNA, etc.) from a complementary nucleotide sequence (e.g., an exon) encoding the nucleotide sequence of the RNA molecule.

Introns are characterized by a set of “splice elements” that are part of the splicing machinery and are required for splicing. Introns are relatively short, conserved nucleic acid segments that bind the various splicing factors that carry out the splicing reactions. Thus, each intron is defined by a 5′ splice site, a 3′ splice site, and a branch point situated therebetween. Splice elements also comprise exon splicing enhancers and silencers, situated in exons, as well as intron splicing enhancers and silencers situated in introns at a distance from the splice sites and branch points. In addition to splice site and branch points, these elements control alternative, aberrant and constitutive splicing.

According to embodiments of this invention, the first nucleotide sequence can be, but is not limited to, a nucleotide sequence encoding a protein or peptide, a nucleotide sequence having enzymatic activity as an RNA (e.g., RNAi), a nucleotide sequence encoding a ribozyme, a nucleotide sequence encoding an antisense sequence and/or a nucleotide sequence encoding a small nuclear RNA (snRNA), in any combination.

The terms “exogenous” and/or “heterologous” as used herein can also include a nucleotide sequence that is not naturally occurring in the nucleic acid construct and/or delivery vector (e.g., virus delivery vector) in which it is contained and can also include a nucleotide sequence that is placed into a non-naturally occurring environment and/or position relative to other nucleotide sequences (e.g., by association with a promoter or coding sequence with which it is not naturally associated). Thus, in some embodiments, the first nucleotide sequence of this invention can encode a protein, peptide and/or RNA of this invention that is exogenous or heterologous (i.e., not naturally occurring, not present in a naturally occurring state and/or modified and/or duplicated) to the cell into which it is introduced. The first nucleotide sequence can also be exogenous or heterologous to the vector (e.g. a viral vector) into which it is placed. Furthermore, the second nucleotide sequence can be exogenous or heterologous to the vector into which it is placed and/or with respect to the first nucleotide sequence with which it is associated as an intron and/or with respect to the cell into which it is placed.

Alternatively, the protein, peptide or RNA encoded by the first nucleotide sequence can be endogenous to the cell (i.e., one that occurs naturally in the cell) but is introduced into and/or is present in the cell as an isolated nucleic acid. By “isolated nucleic acid” is meant a nucleic acid that is substantially or essentially free from components normally found in association with the nucleic acid in its natural state. Such components include other cellular material, culture medium from recombinant production, and/or various chemicals used in chemically synthesizing the nucleic acid.

An “isolated” nucleic acid of the present invention is generally free of nucleic acid sequences that flank the nucleic acid of interest in the genomic DNA of the organism from which the nucleic acid was derived (such as coding sequences present at the 5′ or 3′ ends). However, the nucleic acid of this invention can include some additional bases or moieties that do not deleteriously affect the basic characteristics of the nucleic acid.

By “isolated” protein or peptide of this invention is meant a protein or peptide that is substantially free from components normally found in association with the peptide or protein in its natural state.

A molecule of this invention that imparts a biological function can be a messenger RNA, a protein, a peptide, a ribozyme, RNAi, snRNA, an antisense RNA and the like. Thus, in some embodiments, an RNA that imparts a biological function is an RNA that is translated into a protein or peptide that imparts a biological function or it is an RNA that is translated into, and/or functions directly as, an RNA that imparts a biological function as described herein (e.g., a ribozyme, RNAi, snRNA, an antisense RNA, etc.)

Nonlimiting examples of the nucleic acid of this invention include a nucleic acid comprising, consists essentially of and/or consists of the nucleotide sequence as set forth in SEQ ID NO:1 (plasmid TRCBA-int-luc mut), SEQ ID NO:2 (plasmid TRCBA-int-luc (wt)), SEQ ID NO:3 (plasmid TRCBA-int-luc (657GT)), SEQ ID NO:4 (plasmid GL3-int-Luc (mut)), SEQ ID NO:5 (GL3-int-Luc (wt)), SEQ ID NO:6 (GL3-int-Luc (657GT)), SEQ ID NO:7 (GL3-21nt-fron-sph (mut)), SEQ ID NO:8 (GL3-31nt-2fron-sph (mut)), SEQ ID NO:9 (GL3-int-Luc A (mut)), SEQ ID NO:10 (GL3-int-Luc B)), SEQ ID NO:11 (GL3-int-Luc C), SEQ ID NO:12 (GL3-int-fron (mut)), SEQ ID NO:13 (GL3-21nt-sph (mut)), SEQ ID NO:14 (GL3-21nt-Sph-C), SEQ ID NO:15 (GL3-sint200-sph (mut)), SEQ ID NO:16 (GL3-sint200-sph (657 GT)), SEQ ID NO:17 (GL3-sint425-sph) and/or SEQ ID NO:35 (TRCBA-int-AAT-654CT) in any combination.

Also provided are nonlimiting examples of functional regions of these sequences as described herein (e.g., the intron and coding sequence of SEQ ID NOS:1-17 (i.e., SEQ ID NOS:21-34), an intron comprising the 654C-T mutation (SEQ ID NO:18), a wild type intron (SEQ ID NO:19) an intron comprising the 654C-T mutation and the 657TA-GT mutation (SEQ ID NO:20) and the intron and coding sequence of SEQ ID NO:35 (SEQ ID NO:36). Thus, the nucleic acid of this invention can comprise, consist essentially of an/or consist of one or more than one nucleotide sequence and/or functional region thereof as identified herein as a first nucleotide sequence. Such first nucleotide sequences and/or functional regions can be present in any combination, including repeats of the same nucleotide sequence, in any order and in any position relative to one another and/or relative to other components of the nucleic acid and the nucleic acid construct of this invention.

The nucleic acid of this invention can further comprise a promoter that directs expression of the first nucleotide sequence. Examples of a promoter that can be included in a nucleic acid of this invention and operably associated with a first nucleotide sequence of this invention include, but are not limited to, constitutive promoters and/or inducible promoters, some nonlimiting examples of which include viral promoters (e.g., CMV, SV40), tissue specific promoters (e.g., muscle MCK), heart (e.g., NSE), eye (e.g., MSK) and synthetic promoters (SP1 elements). An example of a promoter of this invention is chicken beta actin promoter (CB or CBA), as described in the Examples herein. The promoter of this invention can be present in any position on the nucleic acid of this invention where it is in operable association with the first nucleotide sequence. One or more promoters, which can be the same or different, can be present in the same nucleic acid molecule, either together or positioned at different locations on the nucleic acid molecule relative to one another and/or relative to a first nucleotide sequence and/or second nucleotide sequence present on the nucleic acid. Furthermore, an internal ribosome entry signal (IRES) and/or other ribosome-readthrough element can be present on the nucleic acid molecule. One or more such IRESs and/or ribosome readthrough elements, which can be the same or different, can be present in the same nucleic acid molecule, either together and/or at different locations on the nucleic acid molecule. Such IRESs and ribosome readthrough elements can be used to translate messenger RNA sequences via cap-independent mechanisms when multiple first nucleotide sequences are present on a nucleic acid molecule of this invention.

In embodiments of this invention wherein a promoter is present on the isolated nucleic acid of this invention, the promoter can be positioned anywhere in the nucleic acid molecule relative to the first nucleotide sequence(s) and/or second nucleotide sequence(s). For example, the second nucleotide sequence(s) can be positioned between the promoter and the first nucleotide sequence. Furthermore, the second nucleotide sequence(s) can be positioned anywhere in the nucleic acid molecule relative to the first nucleotide sequence. For example, the second nucleotide sequence(s) can be positioned before, after and/or within the first nucleotide sequence. In some embodiments, the second nucleotide sequence(s) can be positioned anywhere within the 5′ one/third of the nucleotides of the first nucleotide sequence, anywhere within the middle one/third of the nucleotides of the first nucleotide sequence and/or anywhere within the 3′ one/third of the nucleotides of the first nucleotide sequence. In some embodiments, the second nucleotide sequence(s) can be positioned anywhere between an open reading frame and a poly(A) site in the first nucleotide sequence.

In certain embodiments wherein two or more second nucleotide sequences are present in the isolated nucleic acid of this invention, the second nucleotide sequences can be positioned to be separated by at least about 5, 10, 15, 20, 25, 30, 35, 40, 45, 50, 60, 70, 80, 90, 100, 125, 150, 175, 200, 250, 300, 350, 400, 450, 500, 550, 600, 650, 700, 750, 800, 850, 900 or 1000 nucleotides, including any number of nucleotides between 5 and 1000 not specifically recited herein.

The second nucleotide sequence of the nucleic acid molecule of this invention can comprise, consist essentially of and/or consist of a first set of splice elements defining a first intron that is removed by splicing to produce a first RNA molecule that imparts a biological function in the absence of activity at a second set of splice elements; and a second set of splice elements defining a second intron different from the first intron, wherein the second intron is removed by splicing to produce no RNA molecule and/or a second RNA molecule that does not impart a biological function, when the second set of splice elements is active. In some embodiments, the second nucleotide sequence of this invention can comprise one or more mutations, which can be a substitution, addition, deletion, etc.

Particular but nonlimiting examples of the second nucleotide sequence of this invention can include, but are not limited to, the nucleotide of any of SEQ ID NOs:18-20, 50-71, 74, 75 and 78. Particular examples of an isolated nucleic acid of this invention include, but are not limited to, SEQ ID NOs:1-17 and 21-36. Particular but nonlimiting examples of blocking oligonucleotides of this invention include SEQ ID NOs:37-49, 72, 73, 76, 79 and 80.

In the nucleic acid of this invention, the first intron is the functional intron that is removed by splicing to produce a first RNA molecule that imparts a biological function. The biological function can be imparted directly in embodiments wherein the first nucleotide sequence is a functional RNA and/or imparted indirectly by translation of the first RNA molecule into a protein, peptide or RNA that imparts a biological function. Such a biological function can include a therapeutic effect, including for example, gene therapy for restoration of, and/or increase in, the activity of a protein, peptide and/or RNA that is otherwise defective and/or present in insufficient or low amounts (e.g., to correct a genetic defect that results in a disease or disorder and is responsive to treatment such as gene therapy).

As described herein, when the nucleic acid of this invention is present in an environment wherein splicing can occur and in the absence of a blocking molecule or compound of this invention, the second set of splice elements that define the second intron is active and the second intron is removed, resulting in the absence of production of the first RNA molecule from the nucleic acid. When the second intron is removed, the result can be the production of a second RNA molecule that does not impart a biological function of this invention (i.e., a nonfunctional RNA) and/or no second RNA molecule production at all.

The second nucleotide sequence of the nucleic acid of this invention can be present anywhere on the nucleic acid molecule as a single nucleotide sequence or the second nucleotide sequence can be present on the same nucleic acid molecule as two or more second nucleotide sequences that can be the same or different. Thus, for example, the second nucleotide sequence can be present in multiples of two or more of the same and/or different nucleotide sequences that can be present in tandem, dispersed throughout the nucleic acid molecule at different positions and/or both together (e.g., in tandem) and dispersed.

The nucleic acid of this invention can be present in a vector and such a vector can be present in a cell. Any suitable vector is encompassed in the embodiments of this invention, including, but not limited to, nonviral vectors (e.g., plasmids, poloxymers and liposomes), viral vectors and synthetic biological nanoparticles (BNP) (e.g., synthetically designed from different adeno-associated viruses, as well as other parvoviruses).

It will be apparent to those skilled in the art that any suitable vector can be used to deliver the heterologous nucleic acids of this invention. The choice of delivery vector can be made based on a number of factors known in the art, including age and species of the target host, in vitro vs. in vivo delivery, level and persistence of expression desired, intended purpose (e.g., for therapy or polypeptide production), the target cell or organ, route of delivery, size of the isolated nucleic acid, safety concerns, and the like.

Suitable vectors also include virus vectors (e.g., retrovirus, alphavirus; vaccinia virus; adenovirus, adeno-associated virus, or herpes simplex virus), lipid vectors, poly-lysine vectors, synthetic polyamino polymer vectors that are used with nucleic acid molecules, such as plasmids, and the like.

Any viral vector that is known in the art can be used in the present invention. Examples of such viral vectors include, but are not limited to vectors derived from Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea enation mosaic virus group; Phycodnaviridae; Picornaviridae; Plasmaviridae; Prodoviridae; Polydnaviridae; Potexvirus group; Potyvirus; Poxyiridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites.

Protocols for producing recombinant viral vectors and for using viral vectors for nucleic acid delivery can be found, e.g., in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997).

Nonlimiting examples of vectors employed in the methods of this invention include any nucleotide construct used to deliver nucleic acid into cells, e.g., a plasmid, a nonviral vector or a viral vector, such as a retroviral vector which can package a recombinant retroviral genome (see e.g., Pastan et al., Proc. Natl. Acad. Sci. U.S.A. 85:4486 (1988); Miller et al., Mol. Cell. Biol. 6:2895 (1986)). For example, the recombinant retrovirus can then be used to infect and thereby deliver a nucleic acid of the invention to the infected cells. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naldini et al., Science 272:263-267, 1996), pseudotyped retroviral vectors (Agrawal et al., Exper. Hematol. 24:738-747, 1996), and any other vector system now known or later identified. Also included are chimeric viral particles, which are well known in the art and which can comprise viral proteins and/or nucleic acids from two or more different viruses in any combination to produce a functional viral vector. Chimeric viral particles of this invention can also comprise amino acid and/or nucleotide sequence of non-viral origin (e.g., to facilitate targeting of vectors to specific cells or tissues and/or to induce a specific immune response). The present invention also provides “targeted” virus particles (e.g., a parvovirus vector comprising a parvovirus capsid and a recombinant AAV genome, wherein an exogenous targeting sequence has been inserted or substituted into the parvovirus capsid).

Physical transduction techniques can also be used, such as liposome delivery and receptor-mediated and other endocytosis mechanisms (see, for example, Schwartzenberger et al., Blood 87:472-478, 1996). This invention can be used in conjunction with any of these and/or other commonly used nucleic acid transfer methods. Appropriate means for transfection, including viral vectors, chemical transfectants, or physico-mechanical methods such as electroporation and direct diffusion of DNA, are described by, for example, Wolff et al., Science 247:1465-1468, (1990); and Wolff, Nature 352:815-818, (1991).

Thus, administration of the nucleic acid of this invention can be achieved by any one of numerous, well-known approaches, for example, but not limited to, direct transfer of the nucleic acids, in a plasmid or viral vector, or via transfer in cells or in combination with carriers such as cationic liposomes. Such methods are well known in the art and readily adaptable for use in the methods described herein. Furthermore, these methods can be used to target certain diseases and tissues, organs and/or cell types and/or populations by using the targeting characteristics of the carrier, which would be well known to the skilled artisan. It would also be well understood that cell and tissue specific promoters can be employed in the nucleic acids of this invention to target specific tissues and cells and/or to treat specific diseases and disorders.

A cell comprising a vector and/or nucleic acid of this invention can be any cell that can contain a vector and/or nucleic acid of this invention, including but not limited to cells from muscle (e.g., smooth muscle, skeletal muscle, cardiac muscle myocytes), liver (e.g., hepatocytes), heart, brain (e.g., neurons), eye (e.g., retinal; corneal), pancreas, kidney, endothelium, epithelium, stem cells (e.g., bone marrow; cord blood), tissue culture cells (e.g., HeLa cells) etc., as are well known in the art.

In some embodiments, the nucleic acids of the present invention have a reduced level of “leakiness” when compared with other gene expression regulation systems. By “leakiness” is meant an amount of gene product or functional RNA that is produced when the system is in the “off” position. For example, in some embodiments described herein, the present system is in the “off” position when the nucleic acid of this invention has no contact with a blocking oligonucleotide, small molecule and/or other compound of this invention and thus, the first intron is not being spliced. Leakiness can be a problem inherent in such regulatory systems but the level of leakiness can be less in some embodiments of the present system than in systems known in the art. Thus, the present invention also provides a gene expression regulation system having reduced leakiness in comparison with other gene expression regulation systems, wherein the system comprises a nucleic acid of this invention and/or a vector of this invention. The degree to which leakiness is reduced in the present system in comparison to other systems can be 5, 10, 15, 20, 25, 30, 35, 40, 45, 50, 55, 60, 65, 70, 75, 80, 85, 90, 95, or 100% less than the amount of leakiness observed in art-known systems.

As one example, the amount of leakiness of a system can be determined by employing a reporter gene in the system and detecting the amount of reporter gene product produced when the system is in the “off” position. Any number of assays can be employed to detect reporter gene product, including but not limited to, protein detection assays such as ELISA and Western blotting and nucleic acid detection assays such as polymerase chain reaction, Southern blotting and Northern blotting. Other assays for detection of gene product can include functional assays, e.g., measurement of an amount of biological activity attributed to the gene product. The nucleic acids and methods of the present invention can be employed in comparative assays to demonstrate a reduced level of leakiness in comparison to other known gene regulation expression systems and nucleic acids employed therein.

Further provided herein are various methods of using the nucleic acid, vectors and cells of this invention. In particular, a method is provided herein for producing the first RNA of this invention, comprising; a) contacting a blocking oligonucleotide and/or a small molecule and/or other compound of this invention with the nucleic acid of this invention under conditions that permit splicing, wherein the blocking oligonucleotide and/or small molecule and/or other compound blocks a member of the second set of splice elements, resulting in removal of the first intron by splicing and production of the first RNA.

Additionally provided is a method for producing a protein, comprising: a) contacting a blocking oligonucleotide and/or small molecule and/or other compound of this invention with the nucleic acid of this invention under conditions that permit splicing as would be well known in the art and as described in the examples provided herein, wherein the blocking oligonucleotide blocks a member of the second set of splice elements, resulting in removal of the first intron by splicing and production of the first RNA; and b) translating the first RNA to produce the protein.

In further embodiments, a method is provided for producing an RNA that imparts a biological function, comprising: a) contacting a blocking oligonucleotide and/or small molecule and/or other compound of this invention with the nucleic acid of this invention under conditions that permit splicing, wherein the blocking oligonucleotide and/or small molecule and/or other compound blocks a member of the second set of splice elements, resulting in removal of the first intron by splicing and production of the first RNA; and b) translating the first RNA to produce the RNA that imparts a biological function. In some embodiments, the first RNA can act directly as an RNA that imparts a biological function and in other embodiments the first RNA can be translated into an RNA that imparts a biological function.

In any of the methods described herein, the blocking oligonucleotide and/or small molecule and/or other compound of this invention can be introduced into a cell comprising the nucleic acid of this invention and such a cell can be in an animal, which can be a human, non-human mammal (dog, cat, horse, cow, etc.) or other animal.

A blocking oligonucleotide of this invention is an oligonucleotide (e.g., RNA or DNA or a combination of both) that prevents splicing activity at a specific splice site. Splicing activity is prevented because the blocking oligonucleotide binds to a nucleotide sequence that is a member of the set of splice elements that direct the splicing event, thereby inhibiting the activity of the splice element, resulting in the inhibition of splicing activity. Thus, the blocking oligonucleotide can be complementary to a splice junction, a 5′ splice element, a 3′ splice element, a cryptic splice element, a branch point, a cryptic branch point, a native splice element, a mutated splice element, etc. Some nonlimiting examples of a blocking oligonucleotide of this invention include GCTATTACCTTAACCCAG (SEQ ID NO:37); specific for the 654T mutation of the β globin intron and GCACTTACCTTAACCCAG (SEQ ID NO:38); specific for the 657GT mutation of the β globin intron). Other examples include oligonucleotides comprising, consisting essentially of and/or consisting of the nucleotide sequence of SEQ ID NOs:37, 38, 42, 49, 46, 47, 48, 39, 40, 41, 43, 44, 45, 72, 73, 76, 79 and 80. By “consisting essentially of” in the context of these oligonucleotide sequences, it is intended that the oligonucleotide can include additional nucleotides (e.g., 1, 2, 3, 4, 5, 6, 7, 8, 9, or 10 additional) at either the 3′ end or the 5′ end of the oligonucleotide sequence that do not materially effect the function or activity of the oligonucleotide (e.g., these additional nucleotides do not hybridize to the sequence complementary to the original oligonucleotide sequence).

In methods wherein a blocking oligonucleotide is employed in the methods of this invention, the blocking oligonucleotide can, in some embodiments, be an oligonucleotide that does not activate RNase H. Oligonucleotides that do not activate RNase H can be made in accordance with known techniques. See, e.g., U.S. Pat. No. 5,149,797 to Pederson et al. Such oligonucleotides, which can be deoxyribonucleotide or ribonucleotide sequences, contain any structural modification which sterically hinders or prevents binding of RNase H to a duplex molecule containing the oligonucleotide as one member thereof, which structural modification does not substantially hinder or disrupt duplex formation. Because the portions of the oligonucleotide involved in duplex formation are substantially different from those portions involved in RNase H binding thereto, numerous oligonucleotides that do not activate RNase H are available.

Oligonucleotides of this invention can also be oligonucleotides wherein at least one, or all, of the internucleotide bridging phosphate residues are modified phosphates, such as methyl phosphonates, methyl phosphonothioates, phosphoromorpholidates, phosphoropiperazidates and phosphoramidates. As an additional example, every other one of the internucleotide bridging phosphate residues can be modified as described. In another non-limiting example, such oligonucleotides are oligonucleotides wherein at least one, or all, of the nucleotides contain a 2′ loweralkyl moiety (e.g., C1-C4, linear or branched, saturated or unsaturated alkyl, such as methyl, ethyl, ethenyl, propyl, 1-propenyl, 2-propenyl, and isopropyl). For example, every other one of the nucleotides can be modified as described. (See also Furdon et al., Nucleic Acids Res. 17:9193-9204 (1989); Agrawal et al., Proc. Natl. Acad. Sci. USA 87:1401-1405 (1990); Baker et al., Nucleic Acids Res. 18, 3537-3543 (1990); Sproat et al., Nucleic Acids Res. 17:3373-3386 (1989); Walder and Walder, Proc. Natl. Acad. Sci. USA 85:5011-5015 (1988).) Thus, in some embodiments, the blocking nucleotide of this invention can comprise a modified internucleotide bridging phosphate residue that can be, but is not limited to, a methyl phosphorothioate, a phosphoromorpholidate, a phosphoropiperazidate and/or a phosphoramidate, in any combination. In certain embodiments, the blocking oligonucleotide can comprise a nucleotide having a loweralkyl substituent at the 2′ position thereof.

Additional examples of modified oligonucleotides of this invention include peptide nucleic acids (PNA) and locked nucleic acids (LNA).

In a PNA, the backbone is made from repeating N-(2-aminoethyl)-glycine units linked by peptide bonds. The different bases (purines and pyrimidines) are linked to the backbone by methylene carbonyl linkages. Unlike DNA or other DNA analogs, PNAs do not contain any pentose sugar moieties or phosphate groups. PNAs are depicted like peptides with the N-terminus at the first (left) position and the C-terminus at the right.

The PNA backbone is not charged and this confers to this polymer a much stronger binding between PNA/DNA strands than between PNA strands and DNA strands. This is due to the lack of charge repulsion between PNA and DNA strands.

Early experiments with homopyrimidine strands have shown that the Tₘ of a 6-mer PNA T/DNA dA was determined to be 31° C. in comparison to a DNA dT/DNA dA 6-mer duplex that denatures at a temperature less than 10° C.

PNAs with their peptide backbone bearing purine and pyrimidine bases are not a molecular species easily recognized by nucleases or proteases. They are thus resistant to enzyme degradation. PNAs are also stable over a wide pH range. Because they are not easily degraded by enzymes, the lifetime of these polymers is extended both in vitro and in vivo. In addition, the fact that they are not charged facilitates their crossing through cell membranes and their stronger binding properties should decrease the amount of oligonucleotide needed for the regulation of gene expression.

LNAs are a class of nucleic acids containing nucleosides whose major distinguishing characteristic is the presence of a methylene bridge between the 2′-O and 4′-C atoms of the ribose ring. This bridge restricts the flexibility of the ribofuranose ring of the nucleotide analog and locks it into the rigid bicyclic N-type conformation. Furthermore, LNA induces adjacent DNA bases to adopt this conformation, resulting in the formation of the more thermodynamically stable form of the A duplex LNA nucleosides containing the four common nucleobases that appear in DNA (A,T,G,C) that can base-pair with their complementary nucleosides according to standard Watson-Crick rules. LNA can be mixed with DNA or RNA, as well as other nucleic acid analogs using standard phosphoramidite DNA synthesis chemistry. Therefore, LNA oligonucleotides can easily be tagged with, e.g., amino-linkers, biotin, fluorophores, etc. Thus, a very high degree of freedom in the design of primers and probes exists. Their locked conformation increases binding affinity for complementary sequences and provides a new chemical approach to optimize and fine tune primers and probes for sensitive and specific detection of nucleic acids. This difference is observable experimentally as an increased thermal stability of LNA-NA heteroduplexes and is dependent both on the number of LNA nucleosides present in the sequence, as well as the chemical nature of the nucleobases employed. This experimental difference can be exploited to modulate the specificity of oligonucleotide probes designed to detect specific nucleic acids targets through standard hybridization techniques.

As used herein, “a member of the second set of splice elements” includes any element that is involved in activation of splicing of the second intron. For example, an element of the second set of splice elements can be the result of a mutation in the native DNA and/or pre-mRNA that can be either a substitution mutation and/an addition mutation and/or a deletion mutation that creates a new splice element. The new splice element is thus one member of a second set of splice elements that define a second intron. The remaining members of the second set of splice elements can also be members of the set of splice elements that define the first intron. For example, if the mutation creates a new, second 3′ splice site which is both upstream from (i.e., 5′ to) the first 3′ splice site and downstream from (i.e., 3′ to) a first branch point, then the first 5′ splice site and the first branch point can serve as members of both the first set of splice elements and the second set of splice elements.

In some situations, the introduction of a second set of splice elements can cause native regions of the RNA that are normally dormant, or play no role as splicing elements, to become activated and serve as splicing elements. Such elements are referred to as “cryptic” elements. For example, if a new 3′ splice site is introduced, which is situated between the first 3′ splice site and the first branch point, it can activate a cryptic branch point between the new 3′ splice site and the first branch point.

In other situations, the introduction of a new 5′ splice site that is situated between the first branch point and the first 5′ splice site can further activate a cryptic 3′ splice site and a cryptic branch point sequentially upstream from the new 5′ splice site. In this situation, the first intron becomes divided into two aberrant introns, with a new exon situated therebetween.

Further, in some situations where a first splice element (particularly a branch to point) is also a member of the set of second splice elements, it can be possible to block the first element and activate a cryptic element (i.e., a cryptic branch point) that will recruit the remaining members of the first set of splice elements to force correct splicing over incorrect splicing. Note further that, when a cryptic splice element is activated, it can be situated in either the intron and/or in one of the adjacent exons.

Thus as indicated above, depending on the set of splice elements that make up the “second set of splice elements,” the blocking oligonucleotide, small molecule and/or other compound of this invention can block a variety of different splice elements to carry out the instant invention. For example, it can block a mutated element, a cryptic element, a native element, a 5′ splice site, a 3′ splice site, and/or a branch point. In general, it will not block a splice element which also defines the first intron, of course taking into account the situation where blocking a splice element of the first intron activates a cryptic element which then serves as a surrogate member of the first set of splice elements and participates in correct splicing, as discussed above.

The length of the blocking oligonucleotide (i.e., the number of nucleotides therein) is not critical so long as it binds selectively to the intended location, and can be determined in accordance with routine procedures. Thus, in some embodiments, the blocking oligonucleotide of this invention can be between about 5 and about 100 nucleotides in length. In particular, a blocking nucleotide of this invention can be about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 or 100 nucleotides in length. In some embodiments the blocking oligonucleotide of this invention is from eight to 50 nucleotides in length. In yet other embodiments of this invention, the blocking oligonucleotide is 15-25 nucleotides in length and can also be 18-20 nucleotides in length. A blocking oligonucleotide can be used in a method described herein as a population of identical oligonucleotides and/or as a population of different oligonucleotides present in any combination and/or in any ratio relative to one another.

A small molecule of this invention is an active chemical compound that can be structurally and/or functionally diverse in comparison with other small molecules and that has a low molecular weight (e.g., 55000 Daltons). A small molecule can be a natural or synthetic substance. They can be synthesized by organic chemistry protocols and/or isolated from natural sources, such as plants, fungi and microbes. A small molecule can be “drug-like” (e.g., aspirin, penicillin, chemotherapeutics), toxic and/or natural. A small molecule drug can be one or more active chemical compounds, typically formulated as an orally available pill, that interact with a specific biological target, such as a receptor, enzyme or ion channel, to provide a therapeutic effect. Specific but nonlimiting examples of a small molecule of this invention include antibiotics, nucleoside analogs (e.g., toyocamycin) and aptamers (e.g., RNA aptamers; DNA aptamers).

A small molecule of this invention can be a small molecule present in any number of small molecule libraries, some of which are available commercially. Nonlimiting examples of libraries that can contain a small molecule of this invention include small molecule libraries obtained from various commercial entities, for example, SPECS and BioSPEC B.V. (Rijswijk, the Netherlands), Chembridge Corporation (San Diego, Calif.), Comgenex USA Inc., (Princeton, N.J.), Maybridge Chemical Ltd. (Cornwall, UK), and Asinex (Moscow, Russia). One representative example is known as DIVERSet™, available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan et al. “Stereoselective Synthesis of Over Two Million Compounds Having Structural Features Both Reminiscent of Natural Products and Compatible with Miniaturized Cell-Based Assays” Am. Chem. Soc. 120, 8565-8566, 1998; Floyd et al. Prog Med Chem 36:91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc.

The small molecules and other compounds of this invention can operate by a variety of mechanisms to modify a splicing event in the nucleic acid of this invention. For example, the small molecules and other compounds of this invention can interfere with the formation and/or function and/or other properties of splicing complexes, spliceosomes, and their components such as hnRNPs, snRNPs, SR-proteins and other splicing factors or elements, resulting in the prevention and/or induction of a splicing event in a pre-mRNA molecule. As another example, the small molecules and other compounds of this invention can prevent and/or modify transcription of gene products, which can include, for example, but are not limited to, hnRNPs, snRNPs, SR-proteins and other splicing factors, which are subsequently involved in the formation and/or function of a particular spliceosome. The small molecules and other compounds of this invention can also prevent and/or modify phosphorylation, glycosylation and/or other modifications of gene products, including but not limited to, hnRNPs, snRNPs, SR-proteins and other splicing factors, which are subsequently involved in the formation and/or function of a particular spliceosome. Additionally, the small molecules and other compounds of this invention can bind to and/or otherwise affect specific pre-mRNA so that a specific splicing event is prevented or induced via a mechanism that does not involve basepairing with RNA in a sequence-specific manner.

The present invention further provides a method of producing a protein and/or an RNA that imparts a biological function in a subject, comprising: a) introducing into the subject the nucleic acid, the vector and/or the cell of this invention; and b) introducing into the subject a blocking oligonucleotide and/or small molecule and/or other compound of this invention that blocks a member of the second set of splice elements, thereby producing the protein and/or RNA that imparts a biological function in the subject.

Additionally provided is a method of regulating production of a protein and/or RNA that imparts a biological function in a subject, comprising: a) introducing into the subject the nucleic acid, the vector and/or the cell of this invention; and b) introducing into the subject a blocking oligonucleotide and/or small molecule and/or other compound of this invention that blocks a member of the second set of splice elements, at a time when production of the protein and/or RNA is desired, thereby regulating production of the protein and/or RNA in the subject. The amount of protein and/or RNA present in a subject can be monitored over time according to art-known methods and when the amount falls below a desired and/or therapeutic level, the blocking oligonucleotide, small molecule and/or other compound can be introduced into the subject to increase production of the protein and/or RNA, thus regulating the production.

In the methods described herein wherein the nucleic acid, vector and/or cell of this invention is administered to a subject, the nucleic acid, vector and/or cell can initially be present in the subject in the absence of a blocking oligonucleotide and/or small molecule and/or other compound, the presence of which would result in blocking of a member of the second set of splice elements. In this status, the second set of splice elements is active and, there is no or very minimal (e.g., insignificant) production in the subject of the exogenous protein, peptide and/or RNA that imparts a biological function, as encoded by the first nucleotide sequence. When the blocking oligonucleotide, small molecule and/or other compound of this invention is present in the subject, a member of the second set of splice elements on the nucleic acid is blocked, resulting in removal of the first intron by splicing and subsequent production, in the subject, of the protein and/or RNA encoded by the first nucleotide sequence that imparts a biological function.

The blocking oligonucleotide, small molecule and/or other compound can be introduced into the subject at any time relative to the introduction into the subject of the nucleic acid, vector and/or cell of this invention. For example, the blocking oligonucleotide, small molecule and/or other compound can be introduced into the subject before, simultaneously with and/or after introduction of the nucleic acid, vector and/or cell into the subject. Furthermore, the blocking oligonucleotide, small molecule and/or other compound can be administered one time or at multiple times over any time interval and can extend to throughout the lifespan of the subject.

Thus, in some embodiments, the present invention provides a method of treating a disease or disorder in a subject, comprising: a) introducing into the subject an effective amount of the nucleic acid, vector and/or the cell of this invention; and b) introducing into the subject an effective amount of a blocking oligonucleotide, small molecule, and/or other compound of this invention, thereby treating the disorder in the subject. When the nucleic acid, vector and/or cell and the blocking oligonucleotide, small molecule and/or other compound are present in the subject, they are present under conditions whereby the blocking oligonucleotide, small molecule and/or other compound can contact the nucleic acid and block a member of the second set of splice elements, thereby resulting in the production of a protein, peptide and/or RNA that imparts a biological function in the subject

In additional embodiments of this invention, regulation of gene expression according to the methods of this invention can occur in the reverse of the system described herein. Specifically, in some embodiments of this invention, the system is in the “OFF” position as described herein in the absence of blocking oligonucleotide, small molecule and/or other compound that regulates splice-mediated expression (e.g., no first RNA is produced, leading to the production of a protein, peptide and/or RNA that imparts a biological function). In certain other embodiments, the system of this invention can be in the “ON” position in the absence of blocking oligonucleotide, small molecule and/or other compound that regulates splice-mediated expression. In such latter embodiments, the methods of this invention can be carried out whereby a nucleic acid, vector and/or cell of this invention that is present under conditions that result in the removal of the first intron and production of the first RNA is contacted with a blocking oligonucleotide, small molecule and/or other compound of this invention, resulting in blocking of a member of the first set of splice elements, thereby resulting in the splicing and removal of the second intron, thus producing no second RNA molecule and/or a second RNA molecule that does not impart a biological function.

An “effective amount” of a nucleic acid, vector, cell, blocking oligonucleotide, small molecule and/or other compound of this invention refers to a nontoxic but sufficient amount to provide a desired effect, which can be a beneficial and/or therapeutic effect. As is well understood in the art, the exact amount required will vary from subject to subject, depending on age, gender, species, general condition of the subject, the severity of the condition being treated, the particular agent administered, and the like. An appropriate “effective” amount in any individual case may be determined by one of ordinary skill in the art by reference to the pertinent texts and literature (e.g., Remington's Pharmaceutical Sciences (latest edition) and/or by using routine pharmacological procedures.

“Treat” or “treating” as used herein refers to any type of treatment that imparts a benefit to a subject that is diagnosed with, at risk of having, suspected to have and/or likely to have a disease or disorder that can be responsive in a positive way to a protein and/or RNA of this invention. A benefit can include an improvement in the condition of the subject (e.g., in one or more symptoms), delay and/or reversal in the progression of the condition, prevention or delay of the onset of the disease or disorder, etc.

As noted herein, the present invention provides a method of treating a disorder or disease of this invention comprising: a) introducing into the subject an effective amount of the nucleic acid of this invention; and b) introducing into the subject an effective amount of a blocking oligonucleotide and/or small molecule of this invention, thereby treating the disorder or disease in the subject.

The disease or disorder that can be treated by a method of this invention can include any disease or disorder that is responsive to treatment involving the presence and/or increase in amount in a subject of a protein, peptide and/or RNA of this invention that imparts a biological function. Such proteins, peptides and/or RNAs can be present in a subject via the introduction into the subject of a nucleic acid, vector and/or cell of this invention and introduction into the subject of a blocking oligonucleotide, small molecule and/or other compound of this invention.

Nonlimiting examples of diseases and/or disorders that can be treated by methods of this invention and some examples of the gene product that can be encoded by the first nucleotide sequence of this invention and that can impart a therapeutic effect include metabolic diseases such as diabetes (insulin), growth/development disorders (growth hormone; zinc finger proteins that regulate growth factors), blood clotting disorders (e.g., hemophilia A (Factor VIII); hemophilia B (Factor IX)), central nervous system disorders (e.g., seizures, Parkinson's disease (glial derived neurotrophic factor (GDNF) and GDNF-like growth factors), Alzheimer's disease (nerve growth factor, GDNF and GDNF-like growth factors), amyotrophic lateral sclerosis, demyelination disease), bone allograft (bone morphogenic protein 2 (proteins 1-9, e.g., MBP2)), inflammatory disorders (e.g., arthritis, autoimmune disease), obesity, cancer, cardiovascular disease (e.g., congestive heart failure (phospholamban and genes related to Ca⁺⁺ pump)), macular degeneration (pigment epithelium derived factor (PDEF), β-thalassemia, α-thalassemia, Tay-Sachs syndrome, phenylketonuria, cystic fibrosis and/or viral infection.

Additional examples include nucleic acids encoding soluble CD4, used in the treatment of AIDS and α-antitrypsin, used in the treatment of emphysema caused by a-antitrypsin deficiency. Other diseases, syndromes and conditions that can be treated by the methods and compositions of this invention include, for example, adenosine deaminase deficiency, sickle cell deficiency, brain disorders such as Huntington's disease, lysosomal storage diseases, Gaucher's disease, Hurler's disease, Krabbe's disease, motor neuron diseases such as dominant spinal cerebellar ataxias (examples include SCA1, SCA2, and SCA3), thalassemia, hemophilia, phenylketonuria, and heart diseases, such as those caused by alterations in cholesterol metabolism, and defects of the immune system. Other diseases that can be treated by these methods include metabolic disorders such as, musculoskeletal diseases, cardiovascular disease and cancer. The nucleic acids of this invention can also be delivered to airway epithelia to treat genetic diseases such as cystic fibrosis, pseudohypoaldosteronism, and immotile cilia syndrome, as well as non-genetic disorders (e.g., bronchitis, asthma). The nucleic acids of this invention can also be delivered to alveolar epithelia to treat genetic diseases like α-1-antitrypsin, as well as pulmonary disorders (e.g., treatment of pneumonia and emphysema pulmonary fibrosis, pulmonary edema; delivery of nucleic acid encoding surfactant protein to premature babies or patients with ARDS).

In general, the nucleic acids and vectors of the present invention can be employed to deliver any nucleic acid with a biological function to treat or ameliorate the symptoms associated with any disorder related to gene expression. Illustrative disease states include, but are not limited to: cystic fibrosis (and other diseases of the lung), hemophilia A, hemophilia B, thalassemia, anemia and other blood disorders, AIDS, cancer (e.g., brain tumors), diabetes mellitus, muscular dystrophies (e.g., Duchenne, Becker), Gaucher's disease, Hurler's disease, adenosine deaminase deficiency, glycogen storage diseases and other metabolic defects, mucopolysaccharide disease, and diseases of solid organs (e.g., brain, liver, kidney, heart, lung, eye), and the like.

In certain embodiments, the delivery vectors of the invention may be administered to treat diseases of the CNS, including genetic disorders, neurodegenerative disorders, psychiatric disorders and/or tumors. Illustrative diseases of the CNS include, but are not limited to, Alzheimer's disease, Parkinson's disease, Huntington's disease, Rett Syndrome, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulimia) and cancers and tumors (e.g., pituitary tumors) of the CNS.

Disorders of the CNS that can be treated according to the methods of this invention include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The delivery vectors of the present invention can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors can also be co-delivered, either intraocularly (e.g., intravitreally) or periocularly.

Uveitis involves inflammation. One or more anti-inflammatory factors can be administered by intraocular (e.g., vitreous or anterior chamber) administration of a nucleic acid of the invention.

Retinitis pigmentosa, by comparison, is characterized by retinal degeneration. In representative embodiments, retinitis pigmentosa can be treated by intraocular (e.g., vitreal) administration of a delivery vector encoding one or more neurotrophic factors.

Age-related macular degeneration involves both angiogenesis and retinal degeneration. This disorder can be treated by administering the nucleic acid of this invention encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the inventive delivery vectors. Such agents include N-methyl-D-aspartate (NMDA) antagonists, cytokines, and neurotrophic factors, delivered intraocularly, preferably intravitreally.

In other embodiments, the present invention can be used to treat seizures, e.g., to reduce the onset, incidence and/or severity of seizures. The efficacy of a therapeutic treatment for seizures can be assessed by behavioral (e.g., shaking, ticks of the eye or mouth) and/or electrographic means (most seizures have signature electrographic abnormalities). Thus, the invention can also be used to treat epilepsy, which is marked by multiple seizures over time.

As a further example, somatostatin (or an active fragment thereof) can be administered to the brain using a delivery vector of the invention to treat a pituitary tumor. According to this embodiment, the delivery vector encoding somatostatin (or an active fragment thereof) can be administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins are known in the art.

The present invention also provides methods for screening compounds for the ability to modulate splicing events in the nucleic acids of this invention. Thus, in additional embodiments, the present invention provides a method of identifying a compound that blocks a member of the second set of splice elements of the nucleic acid of this invention, comprising: a) contacting the nucleic acid with the compound under conditions that permit splicing; and b) detecting the production of the first RNA or production of the second RNA, whereby the production of the first RNA identifies a compound that blocks a member of the second set of splice elements of the nucleic acid of this invention and production of the second RNA identifies a compound that to does not block a member of the second set of splice elements. These methods can also be employed to identify compounds that allow for increased or decreased production of the first RNA and/or of the second RNA. Compounds identified by the methods described herein can be employed in the methods of this invention, including methods of producing a protein and/or RNA that imparts a biological function as well as in methods of treatment.

In other embodiments, an alternate splicing event can be modulated by employing the oligonucleotides, small molecules and/or compounds of this invention.

For example, a nucleic acid, vector and/or cell of this invention can be introduced into a subject along with a blocking oligonucleotide, small molecule and/or other compound of this invention to produce a first protein and/or RNA that imparts a biological function in the subject as a result of activation at a particular set of splice sets. The same nucleic acid can be engineered to encode a different protein, peptide and/or RNA that imparts a biological function in the subject by activating a different set of splice sets. The different protein and/or RNA is produced when a different blocking oligonucleotide, small molecule and/or compound of this invention is introduced into the subject. As an example, the first RNA could produce a first protein of interest when a first blocking oligonucleotide, small molecule and/or other compound is present and after addition of a different, second blocking oligonucleotide, small molecule and/or compound of this invention, a second RNA can result, that produces a second protein or functional RNA of interest (e.g., an isoform of the first protein could be produced (e.g., interleukin (IL)-4 and its splice variant, IL-4Δ2). (See, e.g., Fletcher et al. “Increased expression of mRNA encoding interleukin (IL)-4 and its splice variant EL-4Δ2 in cells from contacts of Mycobacterium tuberculosis, in the absence of in vitro stimulation” Immunology 2004 August; 112(4):669-73; Minn et al. “Insulinomas and expression of an insulin splice variant” Lancet 2004 Jan. 31; 363(9406):363-7; Schlueter et al. “Tissue-specific expression patterns of the RAGE receptor and its soluble forms—a result of regulated alternative splicing?” Biochim Biophys Acta 2003 Oct. 20; 1630(1):1-6; Vegran et al. “Implication of alternative splice transcripts of caspase-3 and survivin in chemoresistance” Bull Cancer 2005 March; 92(3):219-26; Ren et al. “Alternative splicing of vitamin D-24-hydroxylase: A novel mechanism for the regulation of extra-renal 1,25-dihydroxyvitamin D synthesis” J Biol Chem. 2005 Mar. 23; et al. “Mutant huntington protein: a substrate for transglutaminase 1, 2, and 3” J Neuropathol Exp Neurol 2005 January; 64(1):58-65; Ding and Keller. “Splice variants of the receptor for advanced glycosylation end products (RAGE) in human brain” Neurosci Lett. 2005 Jan. 3; 373(1):67-72; et al. “Transcript scanning reveals novel and extensive splice variations in human 1-type voltage-gated calcium channel, Cav1.2α1 subunit” J Biol Chem 2004 Oct. 22; 279(43):44335-43, Epub 2004 Aug. 6. All of these references are incorporate by reference herein in their entireties.)

The present invention further provides the nucleic acids, vectors and/or cells of this invention in compositions. Thus, in additional embodiments, the present invention provides a composition comprising the nucleic acid of this invention, the vector of this invention and/or the cell of this invention, in a pharmaceutically acceptable carrier. By “pharmaceutically acceptable carrier” is meant a carrier that is compatible with other ingredients in the pharmaceutical composition and that is not harmful or deleterious to the subject. In particular, it is intended that a pharmaceutically acceptable carrier be a sterile carrier that is formulated for administration to or delivery into a subject of this invention.

Pharmaceutical compositions comprising a composition of this invention and a pharmaceutically acceptable carrier are also provided. The compositions described herein can be formulated for administration in a pharmaceutical carrier in accordance with known techniques. See, e.g., Remington, The Science And Practice of Pharmacy (latest edition). The carrier may be a solid or a liquid, or both, and is preferably formulated with the composition of this invention as a unit-dose formulation, for example, a tablet, which may contain from about 0.01 or 0.5% to about 95% or 99% by weight of the composition. The pharmaceutical compositions are prepared by any of the well-known techniques of pharmacy including, but not limited to, admixing the components, optionally including one or more accessory ingredients.

The pharmaceutical compositions of this invention include those suitable for oral, rectal, topical, inhalation (e.g., via an aerosol) buccal (e.g., sub-lingual), vaginal, parenteral (e.g., subcutaneous, intramuscular, intradermal, intraarticular, intrapleural, intraperitoneal, intracerebral, intraarterial, or intravenous), topical (i.e., both skin and mucosal surfaces, including airway surfaces) and transdermal administration, although the most suitable route in any given case will depend, as is well known in the art, on such factors as the species, age, gender and overall condition of the subject, the nature and severity of the condition being treated and/or on the nature of the particular composition (i.e., dosage, formulation) that is being administered.

Pharmaceutical compositions suitable for oral administration can be presented in discrete units, such as capsules, cachets, lozenges, or tables, each containing a predetermined amount of the composition of this invention; as a powder or granules; as a solution or a suspension in an aqueous or non-aqueous liquid; or as an oil-in-water or water-in-oil emulsion. Oral delivery can be performed by complexing a composition of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the composition and a suitable carrier (which may contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing foim, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions of this invention suitable for parenteral administration can comprise sterile aqueous and non-aqueous injection solutions of the composition of this invention, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The compositions can be presented in unit\dose or multi-dose containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 1 μg to about 10 grams of the composition of this invention. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

Pharmaceutical compositions suitable for rectal administration are preferably presented as unit dose suppositories. These can be prepared by admixing the composition with one or more conventional solid carriers, such as for example, cocoa butter and then shaping the resulting mixture.

Pharmaceutical compositions of this invention suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharmaceutical Research 3:318 (1986)) and typically take the form of an optionally buffered aqueous solution of the composition of this invention. Suitable formulations can comprise citrate or bis\tris buffer (pH 6) or ethanol/water and can contain from 0.1 to 0.2M active ingredient.

An effective amount of a composition of this invention will vary from composition to composition and subject to subject, and will depend upon a variety of factors such as age, species, gender, weight, overall condition of the subject and the particular disease or disorder to be treated. An effective amount can be determined in accordance with routine pharmacological procedures know to those of ordinary skill in the art. In some embodiments, a dosage ranging from about 0.1 μg/kg to about 1 gm/kg will have therapeutic efficacy. In embodiments employing viral vectors for delivery of the nucleic acid of this invention, viral doses can be measured to include a particular number of virus particles or plaque forming units (pfu) or infectious particles, depending on the virus employed. For example, in some embodiments, particular unit doses can include about 10³, 10⁴, 10⁵, 10⁶, 10⁷, 10⁸, 10⁹, 10¹⁰, 10¹¹, 10¹², 10¹³ or 10¹⁴ pfu or infectious particles.

The frequency of administration of a composition of this invention can be as frequent as necessary to impart the desired therapeutic effect. For example, the composition can be administered one, two, three, four or more times per day, one, two, three, four or more times a week, one, two, three, four or more times a month, one, two, three or four times a year and/or as necessary to control a particular condition and/or to achieve a particular effect and/or benefit. In some embodiments, one, two, three or four doses over the lifetime of a subject can be adequate to achieve the desired therapeutic effect. The amount and frequency of administration of the composition of this invention will vary depending on the particular condition being treated or to be prevented and the desired therapeutic effect.

The compositions of this invention can be administered to a cell of a subject either in vivo or ex vivo. For administration to a cell of the subject in vivo, as well as for administration to the subject, the compositions of this invention can be administered, for example as noted above, orally, parenterally (e.g., intravenously), by intramuscular injection, intradermally (e.g., by gene gun), by intraperitoneal injection, subcutaneous injection, transdermally, extracorporeally, topically or the like. Also, the composition of this invention can be pulsed onto dendritic cells, which are isolated or grown from a subject's cells, according to methods well known in the art, or onto bulk PBMC or various cell subfractions thereof from a subject.

If ex vivo methods are employed, cells or tissues can be removed and maintained outside the body according to standard protocols well known in the art while the compositions of this invention are introduced into the cells or tissues. For example, the nucleic acids and vectors of this invention can be introduced into cells via any gene transfer mechanism, such as, for example, virus-mediated gene delivery, calcium phosphate mediated gene delivery, electroporation, microinjection or proteoliposomes. The transduced and/or transfected cells can then be infused (e.g., in a pharmaceutically acceptable carrier) or transplanted back into the subject per standard methods for the cell or tissue type. Standard methods are known for transplantation or infusion of various cells into a subject.

Formulations of the present invention may comprise sterile aqueous and non-aqueous injection solutions of the active compound, which preparations are preferably isotonic with the blood of intended recipient and essentially pyrogen free. These preparations may contain anti-oxidants, buffers, bacteriostats and solutes, which render the formulation isotonic with the blood of the intended recipient. Aqueous and non-aqueous sterile suspensions may include suspending agents and thickening agents. The formulations may be presented in unit dose or multi-dose containers, for example, sealed ampoules and vials, and may be stored in a freeze-dried (lyophilized) condition requiring only the addition of the sterile liquid carrier, for example, saline or water-for-injection immediately prior to use.

In one formulation, the compounds of this invention may be contained within a lipid particle or vesicle, such as a liposome or microcrystal, which may be suitable for parenteral administration. The particles may be of any suitable structure, such as unilamellar or plurilamellar, so long as the compound is contained therein. Positively charged lipids such as N-[1-(2,3-dioleoyloxi)propyl]-N,N,N-trimethyl-ammoniummethylsulfate, or “DOTAP,” are particularly preferred for such particles and vesicles. The preparation of such lipid particles is well known. See, e.g., U.S. Pat. No. 4,880,635 to Janoff et al.; U.S. Pat. No. 4,906,477 to Kurono et al.; U.S. Pat. No. 4,911,928 to Wallach; U.S. Pat. No. 4,917,951 to Wallach; U.S. Pat. No. 4,920,016 to Allen et al.; U.S. Pat. No. 4,921,757 to Wheatley et al.; etc.

The pharmaceutical compositions of this invention can be used, for example, in the production of a medicament for the treatment of a disease and/or disorder as described herein.

The following sequences are included in the present invention.

SEQ ID NO:1. plasmid TRCBA-int-luc mut. Nts 163-2036: CBA promoter; nts. 2739-4573: mutant intron (654 C-T); nts 4592-4813: polyA signal.

SEQ ID NO:2. plasmid TRCBA-int-luc (wt). Nts 163-2036: CBA promoter; nts. 2739-3588: wt intron (654 C); nts 2071-4573: intron in luciferase; nts 4592-4813: polyA signal.

SEQ ID NO:3. plasmid TRCBA-int-luc (657GT). Nts 163-2036: CBA promoter; nts. 2739-3588: mutant intron (654 C-T; 657 TA-GT); nts 2071-4573: intron in luciferase; nts 4592-4813: polyA signal.

SEQ ID NO:4. plasmid GL3-int-Luc (mut). Nts 48-250: SV40 promoter; nts. 948-1797: mutant intron (654 C-T); nts 2814-3035: polyA signal; nts. 280-2782: luciferase with mutant intron.

SEQ ID NO:5. plasmid GL3-int-Luc (wt). Nts 48-250: SV40 promoter; nts. 948-1797: wt intron (654 C); nts 280-2782: luciferase with intron; nts 2814-3035: polyA signal.

SEQ ID NO:6. plasmid GL3-int-Luc (657GT). Nts 48-250: SV40 promoter; nts. 948-1797: intron (654 C-T; 657TA-GT); nts 280-2782: luciferase with mutant intron; nts 2814-3035: polyA signal.

SEQ ID NO:7. plasmid GL3-21nt-fron-sph (mut). Nts 48-250: SV40 promoter; nts. 251-1100; 1771-2620: mutant introns (654 C-T); nts 1103-3635: luciferase with mutant intron; nts 3637-3858: polyA signal.

SEQ ID NO:8. plasmid GL3-31nt-2fron-sph (mut). Nts 48-250: SV40 promoter; nts. 251-1100; 1106-1965; 2635-3484: mutant introns (654 C-T); nts 1967-4469: luciferase with mutant intron; nts 4514-4735: polyA signal.

SEQ ID NO:9. plasmid GL3-int-luc A (mut). Nts 48-250: SV40 promoter; nts. 673-1522: intron (654 C-T); nts 280-2782: luciferase with intron; nts 2814-3035: polyA signal.

SEQ ID NO:10. plasmid GL3-int-Luc B (mut). Nts 48-250: SV40 promoter; nts. 1440-2289: intron (654 C-T); nts 280-2782: luciferase with intron; nts 2814-3035: polyA signal.

SEQ ID NO:11. plasmid GL3-int-Luc C (mut). Nts 48-250: SV40 promoter; nts. 1691-2540: intron (654 C-T); nts 280-2782: luciferase with intron; nts 2814-3035: polyA signal.

SEQ ID NO:12. plasmid GL3-int-fron (mut). Nts 48-250: SV40 promoter; nts. 251-1100: intron (654 C-T); nts 1103-2755: luciferase with intron; nts 2787-3008: polyA signal.

SEQ ID NO:13. plasmid GL3-21nt-sph (mut). Nts 48-250: SV40 promoter; nts. 948-1797; 1798-2647: intron (654 C-T); nts 280-3632: luciferase with intron; nts 3664-3885: polyA signal.

SEQ ID NO:14. plasmid GL3-21nt-sph C (mut). Nts 48-250: SV40 promoter; nts. 948-1797; 2541-3390: intron (654 C-T); nts 280-3632: luciferase with intron; nts 3664-3885: polyA signal.

SEQ ID NO:15. plasmid GL3-sint200-sph (mut). Nts 48-250: SV40 promoter; nts. 948-1597: intron (654 C-T); nts 280-2582: luciferase with intron; nts 2794-2835: polyA signal.

SEQ ID NO:16. plasmid GL3-sint200-sph (657 GT). Nts 48-250: SV40 promoter; nts. 948-1597: intron (654 C-T; 657 TA-GT); nts 280-2582: luciferase with intron; nts 2794-2835: polyA signal.

SEQ ID NO:17. plasmid GL3-sint425-sph. Nts 48-250: SV40 promoter, nts. 948-1373: intron (654 C-T); nts 280-235&: luciferase with intron; nts 2569-2615: polyA signal.

SEQ ID NO:18. mutant intron (654 C-T).

SEQ ID NO:19. wt intron (654 C).

SEQ ID NO:20. intron with two mutations (654 C-T; 657 TA-GT).

SEQ ID NO:21. luciferase cDNA with mutant intron (654 C-T) at nts. 669-1518.

SEQ ID NO:22. luciferase cDNA with wild type intron at nts. 669-1518.

SEQ ID NO:23. luciferase cDNA with double mutant intron (C654 C-T; 657 TA-GT) at nts. 669-1518.

SEQ ID NO:24. luciferase cDNA with mutant intron (654 C-T) at nts. 1-850 and mutant intron (654 C-T) at nts. 1521-2370.

SEQ ID NO:25. luciferase cDNA with mutant intron (654 C-T) at nts. 1-850 and two mutant introns (654 C-T) at nts. 861-1710 and nts. 2385-3234.

SEQ ID NO:26. luciferase cDNA with mutant intron (654 C-T) at alternative location A (nts. 394-1243).

SEQ ID NO:27. luciferase cDNA with mutant intron (654 C-T) at alternative location B (nts. 1161-2010).

SEQ ID NO:28. luciferase cDNA with mutant intron (654 C-T) at alternative location C (nts. 1412-2261).

SEQ ID NO:29. luciferase cDNA with mutant intron (654 C-T) upstream of translation site (nts. 1-850).

SEQ ID NO:30. luciferase cDNA with two mutant introns (654 C-T): at nts. 669-1518 and at nts. 1519-2368.

SEQ ID NO:31. luciferase cDNA with two mutant introns (654 C-T): at nts. 669-1518 and at nts. 2262-3111.

SEQ ID NO:32. luciferase cDNA with mutant intron (654 C-T) at nts. 669-1318 and 200 base pair deletion.

SEQ ID NO:33. luciferase cDNA with double mutant intron (654 C-T; 657 TA-GT) at nts. 669-1318 and 200 basepair deletion.

SEQ ID NO:34. luciferase cDNA with mutant intron (654 C-T) at nts. 669-1094 and 425 basepair deletion.

SEQ ID NO:35. plasmid TRCBA with alpha antitrypsin cDNA and mutant intron (654 C-T) at nts. 2866-3715.

SEQ ID NO:36. alpha antitrypsin cDNA with mutant intron (654 C-T) at nts. 772-1621.

SEQ ID NO:37. blocking oligonucleotide GCT ATT ACC TTA ACC CAG for IVS2-654.

SEQ ID NO:38. blocking oligonucleotide GCA CTT ACC TTA ACC CAG for IVS2-654 with 657GT mutation).

SEQ ID NO:50 (IVS2-654 intron with 564CT mutation). SEQ ID NO:51 (IVS2-654 intron with 657G mutation).

SEQ ID NO:52 (IVS2-654 intron with 658T mutation).

SEQ ID NO:20 (IVS2-654 intron with 657GT mutation). SEQ ID NO:53 (IVS2-654 intron with 200 by deletion).

SEQ ID NO:54 (IVS2-654 intron with 425 by deletion).

SEQ ID NO:68 (IVS2-654 intron with only 197 bp).

SEQ ID NO:69 (IVS2-654 intron with only 247 bp).

SEQ ID NO:55 (IVS2-654 intron with 6A mutation).

SEQ ID NO:56 (IVS2-654 intron with 564C mutation).

SEQ ID NO:57 (IVS2-654 intron with 841A mutation).

SEQ ID NO:58 (IVS2-705 intron).

SEQ ID NO:59 (IVS2-705 intron with 564CT mutation).

SEQ ID NO:60 (IVS2-705 intron with 657G mutation). SEQ ID NO:61 (IVS2-705 intron with 658T mutation).

SEQ ID NO:62 (IVS2-705 intron with 657GT mutation).

SEQ ID NO:63 (IVS2-705 intron with 200 by deletion).

SEQ ID NO:64 (IVS2-705 intron with 425 by deletion).

SEQ ID NO:65 (IVS2-705 intron with 6A mutation).

SEQ ID NO:66 (IVS2-705 intron with 564C mutation).

SEQ ID NO:67 (IVS2-705 intron with 841A mutation).

SEQ ID NO:70 (CFTR exon 19 wild-type sequence).

SEQ ID NO:71 (CFTR exon 19 3849+10 kb C-to-T mutation).

SEQ ID NO:72 (CFTR exon 19 wild-type oligo).

SEQ ID NO:73 (CFTR exon 19 3849+10 kb C-to-T mutation oligo).

SEQ ID NO:74 (Mouse dystrophin intron 22, exon 23 and intron 23 wild-type sequence).

SEQ ID NO:75 (mdx Mouse dystrophin intron 22, exon 23 and intron 23 nonsense mutation).

SEQ ID NO:76 (Antisense exon 23 skipping inducing oligo). SEQ ID NO:39 (oligo for 6A mutation in IVS2-654).

SEQ ID NO:40 (oligo for 564C mutation in IVS2-654).

SEQ ID NO:41 (oligo for 564CT mutation in IVS2-654).

SEQ ID NO:43 (oligo for 841A mutation in IVS2-654).

SEQ ID NO:44 (oligo for 657G mutation in IVS2-654).

SEQ ID NO:45 (oligo for 658T mutation in IVS2-654).

SEQ ID NO:42 (oligo for 705G mutation in IVS2-705). SEQ ID NO:49 (oligo for IVS2-705).

SEQ ID NO:46 (oligo for IVS2-654).

SEQ ID NO:47 (oligo for IVS2-654).

SEQ ID NO:48 (oligo for IVS2-654).

The examples, which follow, are set forth to illustrate the present invention, and are not to be construed as limiting thereof.

### EXAMPLES

### Example 1

### Splicing Mediated Control of Viral Vector Derived Gene Expression

### Construction of Plasmid.

Plasmid pGL3 was purchased from Promega. All primers were obtained from the UNC-CH LCCC oligonucleotide core facility. All enzymes were from New England Biolabs and were used following the vendor's recommendation. To insert wild type (wt) or intron sequence with cryptic splice site(s) in the middle of green fluorescent protein (GFP) or luciferase (Luc) cDNA, insertion sites were chosen according to consensus sequences in pre-mRNAs (Luca Cartegni et al. “Listening to silence and understanding nonsense exonic mutations that affect splicing” Nat Rev Genet. 2002 Apr.; 3(4):285-98).

The intron was inserted into various positions (based on the luciferase cDNA initiation codon ATG numbered 1): 393-394 (A), 668-669 (B), 1160-1161(C), and 1411-1412 (D). In some studies, the intron was inserted between the promoter and the luciferase cDNA. A four-fragment ligation strategy was applied. Pfu enzyme (Stratagen) was used to amplify the intron and both flanking upstream sequences with NcoI and downstream sequence with XbaI by polymerase chain reaction (PCR). The GL3 backbone was digested with both NcoI and XbaI, while flanking the PCR product with either NcoI or XbaI. The intron was inserted by blunt ligation. The segment was purified from a gel. After 1 hr, room temperature ligation by Fast Ligase (Epicentre) was carried out, then the nucleic acid was transformed to DH10B bacterial cells by electroperforation.

### Virus Preparation

AAV2 vectors carrying intron regulated transgene cassettes were made according to a standard 3-plasmid co-transfection procedure (Xiao et al. “Efficient long-term gene transfer into muscle tissue of immunocompetent mice by adeno-associated virus vector” J. Virol. 1996 November; 70(11): 8098-108). The titer was determined by dot blot.

### Luciferase Expression Assay In Vitro

In some experiments, 293 cells were transfected in a 24-well plate. For each well, 10 ng plasmid 5 μl, 2.5M CaCl₂ 10 μl and ddH₂O 85 μl were mixed together before adding 100 μl 2×HeBS. This was added to cells after precipitation formed under light microscopy. Some cells were treated with oligo (e.g., 0.05 mM, 10 μl) at the same time.

After a 24 hour incubation at 37° C., 5% CO₂, the cells were lysed with 100 μl of 1× lysis buffer for each well after washing with 200 μl of 1×PBS. A volume of 20 μl was taken to a 96-well opaque plate for a luciferase assay by using a Microplate Luminometer (Tropix). Luciferase substrate was purchased from Promega.

### Animal Work

One week after virus injection, the animal was anesthetized by intraperitoneal (i.p.) injection of 2.5 mM avertin or isoflurane. Luciferin substrate (125 μl, 25 mg/ml, Promega) was given i.p. to elicit a fluorescence reaction. A Luciferase Imaging System (Roper Scientific) or IVIS imaging system (Xenogen) was applied to capture “real time” imaging of luciferase fluorescence from the whole animal. Images were collected initially (day 0) and then after the oligonucleotide was given for two consecutive days (i.p. 25 mg/kg).

In this example, naturally occurring mutations in the β-globin intron were used to develop a regulated splicing system. These intron mutations were discovered in patients with β-thallasemia and found to cause disease by creating a new 5′ splice donor site. The new donor site, in cooperation with a cryptic 3′ splice acceptor, results in the inclusion in the mRNA of a portion of the intron carrying an in-frame stop signal.

Specifically in this example, it is demonstrated that the mutated intron, included in the green fluorescent protein (GFP) transgene of an AAV vector, can be used as a complete vector regulation system. Addition of an oligonucleotide (“oligo”) directed to the mutation corrects the splicing defect and induces correct gene expression both in vitro and in vivo.

AAV plasmid vectors were constructed by cloning a green fluorescent protein (GFP) or luciferase reporter gene containing a wild type or mutant β-globin intron incorporated behind either a human cytomegalovirus (CMV) promoter or a hybrid CMV chicken β-actin promoter (CB or CBA). Two different splice mutations were incorporated into separate AAV vectors, a mutation at nt 654 of the intron (AAV-654) and a mutation at nt 705, which has an additional mutation in the cryptic splice site (AAV-705U). Transfection of the AAV constructs into HEK 293 cells or HeLa cells resulted in good gene expression with the wild type intron and low gene expression with mutant introns. Subsequent transfection of the cells with a 2′-O-methoxyethyl phosphorothioate (MOE) oligonucleotide directed to the mutation at nt 654 or to the mutation at nt 705, respectively, increased gene expression from the 654 and 705U mutants, respectively.

Recombinant AAV was generated and tested in both HEK 293 cells and HeLa cells. Twenty-four hours after AAV infection, cells were transfected with an MOE oligonucleotide directed to the corresponding mutation and reporter gene expression was observed at 24 and 48 hours post-oligo transfection. Cells infected with AAV-654 or AAV-705U and without oligo demonstrated virtually no GFP expression at 24 hours post-transfection and only slight gene expression at 48 hours. In contrast, cells transfected with oligo demonstrated significant gene expression at 24 hours, which increased somewhat in intensity, but not in number of cells at 48 hours. Counts of GFP positive cells indicated up to a 200 fold induction with the addition of oligo for the 654 mutant and a 70 fold induction for the 705 mutant at 48 hours. The 705U mutant demonstrated less robust induction in HeLa cells and in HEK 293 cells, as assayed by numbers of GFP fluorescent cell counts and by whole field fluorescence. This appeared to be due to a slightly higher basal level of gene expression as well as less robust response to addition of oligo.

Infection with rAAV containing the wild type intron (AAV-wt int) consistently gave strong GFP expression in nearly 100% of the cells at the same multiplicity of infection (MOI) as the mutants. The AAV-wt intron demonstrated significantly more gene expression than either of the mutants in the presence of oligo, indicating incomplete correction by the oligo. Semi-quantitative RT-PCR confirmed both correctly spliced and incorrectly spliced species in both AAV-654 and AAV-706U infected cells in the presence of oligo. However, increasing the dose of oligo did not substantially increase gene expression. Increasing the amount of virus did increase the whole field intensity somewhat, but not the number of GFP positive cells.

Table 1 shows the correction efficiency of one intron in different places relative to luciferase cDNA.

Table 2 shows luciferase transgene expression change with insertion of multiple introns.

Table 3 shows the transgene correction efficiency of an intron that was shortened by one quarter of the original length by deleting base pairs 151-350(SEQ ID NO:53).

### Example 2

### In Vivo Studies

Induction of AAV mediated gene expression by oligonucleotides was also investigated in vivo with the 654 mutant intron construct driven by the CB promoter (AAV-CB-654). An rAAV type 2 vector (5×10¹⁰ vector particles) carrying the 654 mutant intron within a luciferase reporter gene was delivered into mouse liver by portal vein injection. One year later, oligo was given intraperitoneally, 25 mg/kg daily, for 2 days. Luciferase imaging was carried out on day 3. When compared to animals that did not receive oligo treatment, luciferase expression was 8-10 fold higher. The oligo-induced up-regulation observed in vivo persisted for over 1 month and than declined back down to base line level. A second set of animals given vector for 1 week followed by oligo resulted in characteristic up-regulation of transgene expression, followed by decline over 1 month. Repeat administration with oligo could also re-activate intron-regulated transgene expression. This result demonstrates that a vector-specific constitutive promoter is expressing mRNA over an extended period of time (consistent with AAV mediated transgene expression in vivo), but functional gene product is only observed after “splice mediated” drug (e.g., oligonucleotide) is administered.

These results demonstrate the regulation of functional gene expression by regulating splicing of the vector produced RNA from non-functional mRNA to functional mRNA.

Addition of the oligo induced gene expression quite rapidly, generating expression by 24 hours in tissue culture and within 1 to 2 days in vivo. Duration of gene expression is influenced by the half-life of the protein produced by the transgene and the half-life of the oligonucleotide. An oligonucleotide such as 2′-β-methoxyethyl phosphorothioate backbone has a long half-life in vivo; completely intact after 8 hours in the rat. Continued mRNA correction and protein expression could last for quite some time with a single injection of MOE or LNA oligo. It should be possible to alter the duration of gene correction by altering the backbone of oligonucleotides as well as the dose. Different backbones have demonstrated significantly different biostabilities and could be used to more precisely control gene expression duration. The half-life of target mRNA can also be controlled by including cis-acting elements that will cause spliced mRNA to have a fast or slow turn over rate. The use of such elements is standard in the field and familiar to one skilled in the art. Addition of a strong poly A signal will also influence the half-life of the processed message. Therefore, the ability of “splice mediated” drug to up-regulate functional mRNA can be influenced by amount given, bio-distribution, stability and/or affinity for target sequence, as well as by abundance and stability of target mRNA. All of these parameters could be modified according to methods known in the art to more precisely control “splice mediated” regulation.

By using an intron to regulate gene expression, the need for addition of foreign proteins other than the transgene is eliminated, thus avoiding a potential for a serious immune reaction to the regulatory transactivator. In addition, the intron can vary in size (1000 by or less), and can easily be combined with tissue specific promoters, generating tissue specificity and protein expression regulation in a single vector after addition of oligo. In more conventional regulation systems, this generally requires two vectors and two separate promoters (i.e., a regulated promoter to drive transgene expression and a tissue specific promoter to drive the transactivator).

To further demonstrate the utility of this system, a functional therapeutic transgene (alpha 1-antitrypsin; AAT) was cloned into an AAV vector with the intron regulated gene cassette system. After portal vein injection of vector particles, functional AAT transgene activity was measured over time by ELISA assay. In the absence of “splice mediated” oligo, low to no human AAT was detected. However, in the presence of drug (in this example, LNA oligo), up-regulation of transgene expression (100 fold) could be monitored in blood with similar kinetics and duration as described for reporter gene (over 30 days). Consistent with AAV vectors, after vector delivery, transgene expression will ensue and persist regardless of gene cassette (reporter or therapeutic) in target tissue. With respect to “splice mediated” controlled vectors, all aspects of vector delivery are identical with the exception of expression of functional mRNA. This aspect is controlled solely by the presence of exogenous “splice mediating” drug and can be given only at chosen times and/or repeatedly to achieve a desired functional activity of the transgene mRNA.

### Example 3

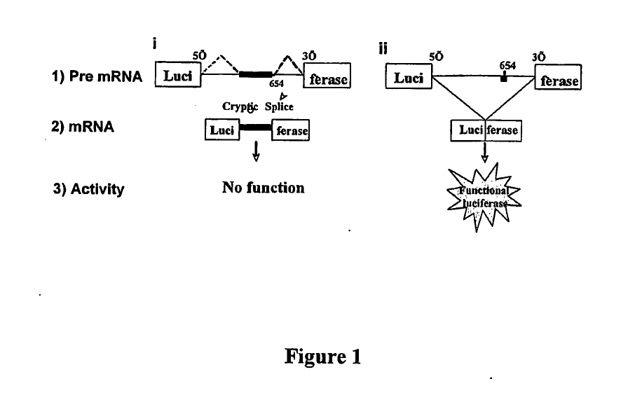

### Studies Depicted in FIGS. 1-3

In some embodiments of this invention, AAV plasmid vectors were constructed by cloning reporter gene cassettes (green fluorescent protein-GFP or luciferase-Luc) containing a mutant β-globin intron within the coding sequence behind either a human cytomegalovirus (CMV) promoter or a hybrid CMV chicken β-actin promoter (CB). AAV vector was generated according to a standard 3-plasmid co-transfection procedure (Xiao et al. Journal of Virology (1998)). Based on the presence of the intron mutant sequence, RNA expression from these vector cassettes results in formation of pre-mRNA (FIG. 1(1)). In the absence of exogenous oligonucleotide, the pre-mRNA will splice using cryptic splice sites. This is a result of a single point mutation located at nt 654 of the intron that results in formation of alternative splice sites (small triangles above the pre-mRNA in FIG. 1(1)(i)). Spliced mRNA generated from this reaction contains a portion of the intron sequence between the two coding sequences (FIG. 1(2)(i)). This mRNA is non-functional and does not express a functional product (FIG. 1(3)(i)). Subsequent transfection of a 2′-O-methoxyethyl phosphorothioate (MOE) oligonucleotide directed to the mutation at nt 654 (right side of black bar in FIG. 1(1)(ii)), blocks alternative splicing, resulting in correct splicing (FIG. 1(2)(ii) and functional gene product (FIG. 1(3)(ii)).

Recombinant AAV vectors carrying the above cassettes were generated and tested for regulated transgene expression in human cells (HeLa cells). Twenty-four hours after AAV infection, ½ of the cells were transfected with an MOE oligonucleotide directed to the 654 mutation and reporter gene expression was observed at 48 hours post-oligo transfection. Cells infected with AAV-654 vector without oligonucleotide demonstrated virtually no detectable GFP expression. In contrast, cells transfected with 654-specific oligonucleotide demonstrated significant gene expression. Counts of GFP positive cells indicated up to a 200-fold induction with the addition of the oligonucleotide for the 654 mutant.

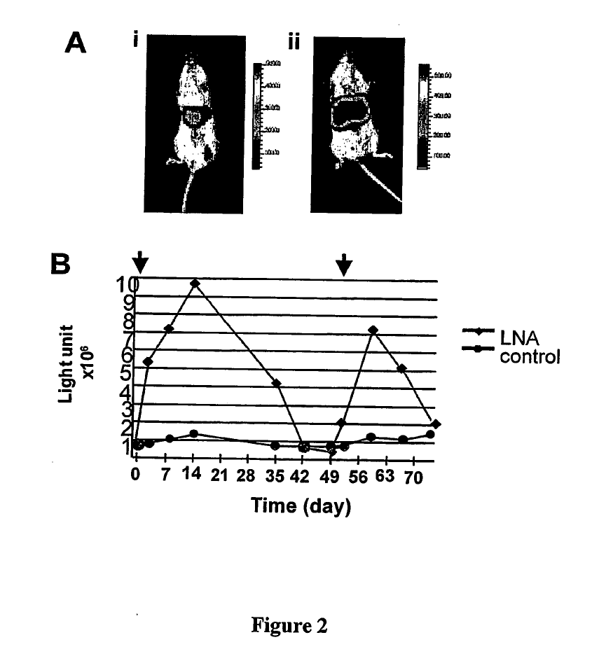

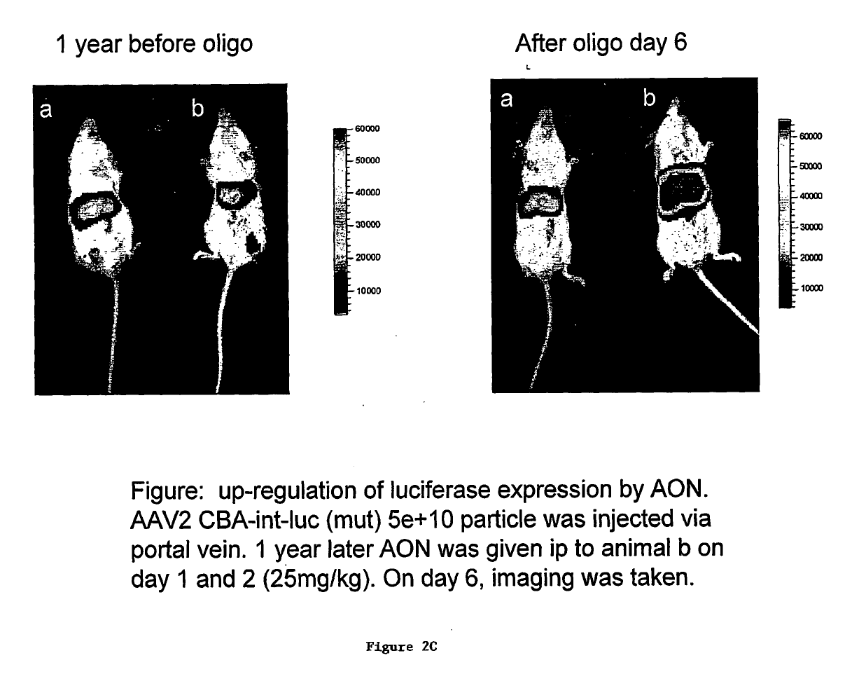

An AAV vector carrying a luciferase reporter gene controlled by a “splice mediated” intron was produced as described herein and used to infect mouse liver by portal vein injection. In one set of animals, vector was administered one year prior to delivery of oligo drug (FIG. 2A). After administration of a splice specific oligo for 2 consecutive days via intraperitoneal injection, real time imaging was performed on animals after injection of luciferin substrate to detect functional luciferase activity by emission and collection of photons (and conversion to light units). As illustrated in FIG. 2A, the mouse receiving oligo (FIG. 2A(ii) and FIG. 2C) displayed increased luciferase activity (Dark grey shading and increased amount of surface area) when compared to a non-treated animal (FIG. 2A(i) and FIG. 2C). These results also demonstrated that a vector specific constitutive promoter is expressing non-functional mRNA and this activity has persisted for over one year. Only after addition of a “splice mediated” oligo does the non-functional mRNA convert to functional mRNA as described in FIG. 1.

In another set of animals infected with “splice mediated” vector transgene cassettes, regulation was induced after oligo administration and persisted for over one month, with steady decline back to base line. Repeat administration of oligo (FIG. 2B arrows) demonstrated up-regulation of transgene activity consistent with first drug administration (FIG. 2B; diamonds). No evidence of up-regulation was observed in animals not receiving “splice mediated” oligo drug (FIG. 2B; black circles). These experiments demonstrate that the vector-delivered transgene cassette is responsive in vivo to the presence of, and sensitive to the duration of, the oligo drug. Numerous experimental parameters related to drug delivery can be modulated by one skilled in the art to influence the level and duration of regulated transgene function (e.g., dose and bio-distribution of drug, half life of drug and target mRNA, stability of mRNA product, etc.).

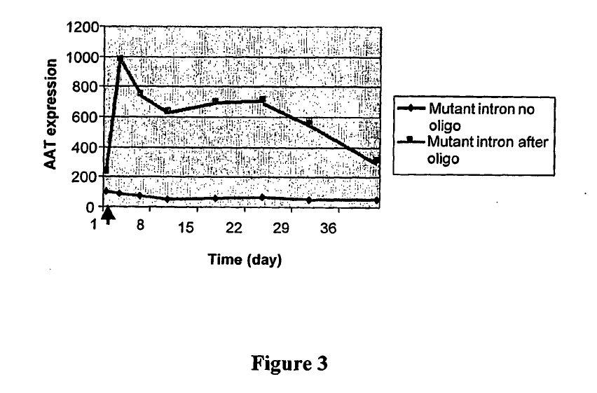

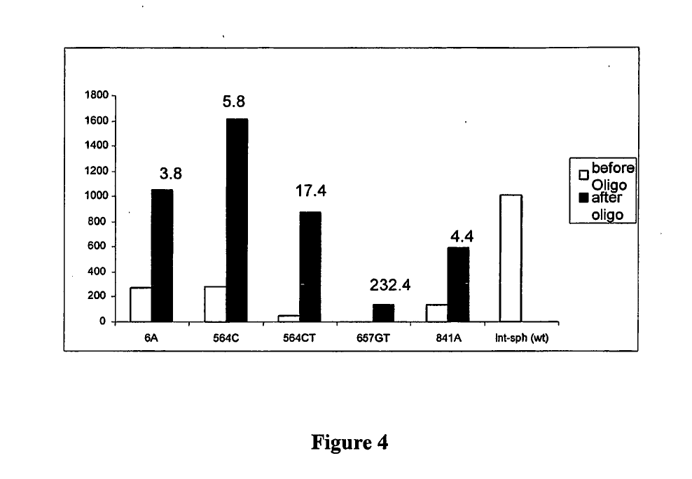

Similar experiments were conducted in an in vivo study using an AAV vector carrying a regulated therapeutic transgene (alpha 1-antitrypsin; AAT). In this example, AAV vector was given by portal vein injection to mouse liver. After one week, subsets of animals were given LNA oligo administered by intraperitoneal injection, followed by measuring circulating levels of AAT protein by ELISA assay. AAT expression peaked at around one week (FIG. 3; squares) and slowly declined over one month. In animals that received vector only (FIG. 3; diamonds), no evidence of AAT expression was observed above base line for the duration of the experiment. Two critical factors primarily determine the longevity of the induced transgene in this experiment; namely half-life of the oligo and protein product, respectively. Different results can be achieved depending on the type of oligo used (PNA vs LNA, etc.) and the transgene being targeted for regulation (AAT vs growth factor vs cytokine, etc.). Regardless, these over all results mimic those of a “splice mediated” regulated reporter gene and demonstrate the ability to regulate therapeutic transgene expression in vivo after exogenous administration of drug via a “splicing mediated” regulated mechanism.

### Example 4

### Double Intron System

Use of alternative splicing for controlling transgene expression in vitro and in vivo. The aberrantly spliced mutated intron of the human β-globin gene, IVS2-654 was inserted into a green fluorescent protein (GFP) expression cassette.

The IVS2-654 intron is 850 by in size and contains four splice sites. The nucleotide sequences of the IVS2-654 intron (SEQ ID NO:19) is shown below. The two alternative introns are located at nucleotides 1-579 and 653-850. The alternative exon is located at nucleotides 580-652. The two arrows mark the junctions between the alternative intron-exon. The four splice sites and the four potential branch sites are indicated by straight and curvy underlines, respectively. The target sequences of the 5′ss 652/18 AON are in bold emboss. Sequences required for efficient splicing and 3′ end formation are in bold italic.

| | |
|---|---|
| 1 | [symbol] [symbol] [symbol] [symbol] [symbol] [symbol] [symbol] CCCTTCTT TTCTATGGTT |
| | AAGTTCATGT CATAGGAAGG GGAGAAGTAA CAGGGTACAG |
| 91 | TTTAGAATGG GAAACAGACG AATGATTGCA TCAGTGTGGA AGTCTCAGGA |
| | TCGTTTTAGT TTCTTTTATT TGCTGTTCAT AACAATTGTT |
| 181 | TTCTTTTGTT TAATTCTTGC TTTCTTTTTT TTTCTTCTCC GCAATTTTTA |
| | CTATTATACT TAATGCCTTA ACATTGTGTA TAACAAAAGG |
| 271 | AAATATCTCT GAGATACATT AAGTAACTTA AAAAAAAACT TTACACAGTC |
| | TGCCTAGTAC ATTACTATTT GGAATATATG TGTGCTTATT |
| 361 | TGCATATTCA TAATCTCCCT ACTTTATTTT CTTTTATTTT TAATTGATAC |
| | ATAATCTTTA TACATATTTA TGGGTTAAAG TGTAATGTTT |
| 451 | TAATATGTGT ACACATATTG ACCAAATCAG GGTAATTTTG CATTTGTAAT |
| | TTTAAAAAAT GCTTTCTTCT TTTAATATAC TTTTTGTTT |
| 541 | ATCTTATTTC TAATACTTTC CCTAATCTCT TTCTTTCAG↓G GCAATAATGA |
| | TACAATGTAT CATGCCTCTT TGCACCATTC TAAAGAATAA |
| 631 | CAGTGATAAT TTCTGGGTTA AG↓GTAATAGC AATATTTCTG CATATAAATA |
| | TTTCTGCATA TAAATTGTAA CTGATGTAAG AGGTTTCATA |
| 721 | TTGCTAATAG CAGCTACAAT CCAGCTACCA TTCTGCTTTT ATTTTATGGT |
| | TGGGATAAGG CTGGATTATT CTG [symbol] [symbol] [symbol] |
| 811 | [symbol] [symbol] [symbol] [symbol] [symbol] [symbol] [symbol] [symbol] |

The resulting plasmid was transfected into 293 cells, a human kidney epithelial cell line, by using the calcium phosphate transfection method. Subsequently, a specific AON at a final concentration of 0.5 μM was added to one of the two identical sets of the transfected cells to induce GFP expression. The specific AON, named 5′ss 652/18 AON, is an 18-mer oligonucleotides complementary to the 5′ alternative splice site and is capable of inhibiting the inclusion of the aberrant exon. As a positive control, 293 cells were separately transfected with a plasmid containing the wild type intron inserted at the same site in the GFP expression cassette. The positive control cells were not treated with the 5′ss 652/18 AON. Twenty-four hours after the transfection, the cells were examined for GFP expression using fluorescence microscopy. In the experimental group, cells transfected but not treated with the AON failed to express a detectable level of GFP. In contrast, the cells treated with the AON expressed functional GFP at a level similar to that of the positive control group. Therefore, alternative splicing could be used to control transgene expression in vitro.

To determine whether alternative splicing could also be used to control transgene expression in vivo, a recombinant AAV plasmid carrying a luciferase expression cassette (Promega) inserted with one copy of the 850 by IVS2-654 intron was constructed. The luciferase gene was driven by the CMV enhancer/chicken R-aclin promoter that had been shown to be able to drive constitutive transgene expression in mice. AAV was produced by utilizing an adenovirus-free production scheme, which involved transfection of 293 cells with three plasmids: the recombinant AAV plasmid, an AAV-helper plasmid which supplies both the structural and the non-structural AAV genes, and an adenovirus-helper plasmid which supplies the essential helper genes for AAV vector production. The resulting AAV vector was purified by utilizing a purification protocol which contained an iodixanol gradient and a heparin sulfate chromatography steps. Then, 5×10¹⁰ particles of the purified AAV were administered into each mouse. One week post injection, luciferase expression was induced by intraperitoneal injection of the 5′ss 652/18 AON at 25 mg/kg daily for 2 consecutive days. The level of luciferase expression was determined by whole body imaging using a Luciferase Imaging System (Roper Scientific) after luciferin administration. When the AAV was targeted to the liver by portal vein injection, luciferase expression in the organ was induced up to 10.4 fold, peaking at day 8 and lasting more than 29 days. AAV targeted to the heart by direct injection also showed a similar pattern of induced transgene expression. AON was also administered to the mice one year after AAV injection, and luciferase expression in the liver was induced to a similar level, indicating that incorporating the intron into an AAV vector did not affect the persistence of the AAV genome.

To more accurately quantify the level of transgene expression and to determine whether alternative splicing could control the expression of other genes of interest in vivo, another AAV vector carrying an al-antitrypsin (AAT) expression cassette inserted with one copy of the 850 by IVS2-654 intron was constructed. The resulting purified AAV was administered to mice via portal vein injection. AAT expression was induced by administration of the 5′ss 652/18 AON and quantified by an ELISA assay. Similar to the pattern of luciferase expression, AAT expression was induced up to 8.9 fold peaking at day 8 and 29 and lasting more than 43 days. These results indicate that alternative splicing can be used to control transgene expression both in vitro and in vivo.

Optimization of alternative splicing for controlling transgene expression. To facilitate the optimization of the alternative splicing for controlling transgene expression, the firefly luciferase marker gene was used for the insertion of the 850 by alternatively spliced intron IVS2-654. Thus, control of transgene expression could be conveniently determined by assaying the levels of luciferase expression under the conditions for both exon inclusion and exon skipping, i.e., in the presence or absence of the 5′ss 652/18 AON. To optimize this alternative splicing for controlling transgene expression, the following three sets of experiments were performed:

1) Insertion of a single copy of the IVS2-654 intron in the luciferase expression cassette to control the transgene expression. To determine whether the insertion site affects the splicing of the intron, a single copy of the 850 by IVS2-654 intron was inserted in between nucleotides 393-394 (A), 668-669(B), 1160-1161(C) or 1411-1412(D), as well as immediately upstream of the translation start (F), i.e., at positions A, B, C, D and F of the luciferase expression cassette. The reason for inserting the intron upstream of the coding sequences is that the aberrant exon itself contains both an upstream ATG start codon and a downstream TAA stop codon. Therefore, inclusion of the aberrant exon at position F should prevent the synthesis of the luciferase protein. The resulting plasmids were separately transfected into 293 cells by using the calcium phosphate transfection method. Free 5′ss 652/18 AON at a final concentration of 0.5 μM was subsequently added to one of the two identical sets of the transfected cells. Twenty-four hours after the transfection, the cells were harvested for quantification of luciferase expression. For intron insertions at positions A-D, the actual levels of luciferase expression varied significantly up to 3.8-fold under the same conditions, i.e., either in the absence or presence of the AON. However, the induction levels for the four constructs were similar, from 4.0 to 5.7 fold. The similarity in the induction level for constructs A-D suggested that flanking sequences did not dramatically influence the alternative splicing. Insertion at position F surprisingly yielded a low induced level of expression and a relatively high background level of expression. The low induced level could be because recognition of the 5′ alternative splice site was enhanced by the 5′ cap structure, resulting in more efficient exon inclusion. The high background level could be due to translation initiated at the correct start codon.

Because the luciferase expression system enables the convenient quantification of both the induction level and the actual expression level, a side-by-side comparison of the alternative splicing approach with the self-cleaving ribozyme approach (38) was carried out. A single copy of the 83 by N79 ribozyme was inserted upstream of the Kozak sequence and the ATG start codon of the luciferase expression cassette. The resulting plasmid and construct C were separately transfected into 293 cells by using the calcium phosphate transfection method. For the ribozyme containing construct, toyocamycin at a final concentration of 1.5 μM was added to one of the two identical sets of the transfected cells. For the intron containing construct, free 5′ss 652/18 AON at a final concentration of 0.5 μM was added to one of the two identical sets of the transfected cells. Twenty-four hours after the transfection, the cells were harvested for quantification of luciferase expression. The induction levels for the intron and ribozyme containing constructs were 5.3 and 1.8 fold, respectively. Additionally, the actual luciferase expression level for the ribozyme-containing construct was 0.4% of that for the intron containing construct. The lower level of luciferase expression for the ribozyme containing construct is consistent with the notion that placement of an AUG-containing ribozyme upstream of the translation start would lead to either inhibition of the correct translation or synthesis of a mutant protein. The higher level of luciferase expression for the intron containing construct was likely due to more efficient formation of the 3′ end of the mRNA in the presence of the intron sequences. It should be clarified that the approximately 260-fold induction of luciferase expression reported for the ribozyme approach was based on a stable cell line carrying two copies of the N79 ribozyme inserted in the luciferase expression cassette (38).

2) Insertion of two copies of the IVS2-654 intron in the luciferase expression cassette to control transgene expression. The purposes of this set of experiments were to test whether inserting two copies of the intron would improve the induction level of transgene expression and whether the distance between the two introns has any effect on the induction level. Therefore, two copies of the IVS2-654 intron with a combined size of 1,700 by were placed at two different sites with various distances in between (AB, AC, AD, BC, BD and FB) or at one site in tandem (BB). The resulting plasmids were separately transfected into 293 cells by using the calcium phosphate transfection method. Free 5′ss 652/18 AON at a final concentration of 0.5 μM was subsequently added to one of the two identical sets of the transfected cells. Twenty-four hours after the transfection, the cells were harvested for quantification of luciferase expression. All constructs except BB led to significantly reduced levels of background expression. As a result, the induction levels were greatly improved, ranging from 10.1 to 143.3 fold. The induction levels were nearly in reverse correlation to the distance between the two introns except in the case of two introns in tandem, i.e., the BB construct. The reduced levels of background expression and therefore the improved induction level of transgene expression when two copies of the intron were in close proximity to a certain extent could be because recognition of the alternative splice sites was enhanced and/or nonsense-mediated decay of the mRNA was accelerated. Nonsense-mediated mRNA decay is a surveillance pathway that reduces errors in gene expression by eliminating aberrant mRNAs that encode incomplete polypeptides. For the BB construct, the background level of expression was significantly higher than the rest of the group. The higher level of background expression was probably because the 3′ splice site of the upstream intron and the 5′ splice site of the downstream intron were too close to each other such that recognition of the splice sites were impaired. Consequently, the two outer most splice sites could become the dominant sites recognized. These results indicate that inserting multiple copies of the intron could improve the induction level of transgene expression. They also indicated that there may be an optimal distance between introns that would yield the highest level of induction.

3) Mutation of the alternative splice sites of the IVS2-654 intron to modulate the alternative splicing. The alternative splice sites in the 850 by IVS2-654 intron were mutated to alter their strength. The first experiment involved knocking our one of the two potential branch points in the upstream alternative intron in construct B. The AA at nucleotides 564 and 565 was converted to CT to make the upstream potential branch point less similar to the consensus sequences. The resulting plasmid was transfected into 293 cells by using the calcium phosphate transfection method. Free 5′ss 652/18 AON at a final concentration of 0.5 μM was subsequently added to an identical set of the transfected cells. Twenty-four hours after the transfection, the cells were harvested for quantification of luciferase expression. The AACT mutation increased the induction level from 4.3 to 13 fold while retaining a relatively high level of induction of transgene expression. This is consistent with the current thinking that use of branch site is one of the mechanisms regulating alternative splicing. The second experiment was designed to optimize alternative splicing by converting the T at nucleotide 657 to G, the A at nucleotide 658 to T, or both the TA to GT in construct B. The mutations were to increase the strength of the alternative 5′ splice site by making the splice site more similar or identical to the consensus sequences. The two constructs with a single base conversion in the splice site both yielded an approximately two-fold increase in the induction levels. Meanwhile, the two-base conversion resulted in a 55-fold increase in the level of induction. The increase in the level of induction was apparently due to a more dramatic decrease in the background level of transgene expression than in the induced level of transgene expression. These results suggested that by modulating the use of a branch site as well as the strength of alternative splice site, alternative splicing could be optimized to control transgene expression.

Development of small introns for alternative splicing. The IVS2-654 intron is 850 base-pairs (bp) long. This size could prove to be a problem for inserting multiple copies of the intron to control transgene expression mediated by AAV. This is because the packaging limit of AAV is 4.7 kb. To minimize the size of the intron, a 200 by fragment, nucleotides 151 to 350, was deleted from the intron in construct B, resulting in construct BΔ200. The sequences have not been shown to play a role in the intron splicing. Construct BΔ200 did not have a decrease in the induction level when compared to construct B. 197 by intron was also derived from IVS2-654, which contained the four essential splice sites and a modified alternative exon, as well as the first 32 by on the 5′ end and the last 57 by on the 3′ end that are required for the efficient splicing and formation of the 3′ end of the β-globin mRNA. Insertion of the 197 by intron into the luciferase gene resulted in alternative splicing of the message, although the induction level was decreased when compared to that for construct B. These results showed that the IVS2-654 intron could be shortened without significantly affecting the induction level.

Generation of transgenic mice carrying a luciferase expression cassette containing an alternative splicing intron. Transgenic mice carrying a firefly luciferase expression cassette inserted with a single copy of the original 850 by IV2-654 intron were generated. Successful delivery of the specific AON for IVS2-654 would inhibit exon inclusion and induce exon skipping, therefore resulting in translation of functional luciferase protein. Thus, whole body imaging of luciferase expression could be conveniently used to monitor the delivery of the AON. Because the transgenic mice assay system does not require labeling of the AON or sacrificing the experimental mice, it would greatly facilitate the optimization of AON delivery. The successful induction of luciferase expression in the transgenic mice following administration of the AON demonstrated the feasibility of using AON delivery and regulating transgene expression in vivo.

Further optimization of the alternative splicing intron. Inserting two copies of IVS2-654 intron into the same expression cassette remarkably reduced the background level of transgene expression and increased the induction level. However, because the size of AAV genome that can be efficiently packaged is limited at 4.7 kb, inserting multiple copies of the 850 by IVS2-654 intron would significantly reduce the cloning capacity of the AAV vector. Shortening the IVS2-645 intron by deleting a 200 by fragment resulted in a similar induction level of transgene expression, and deriving a small 197 by intron from the IVS2-654 intron still retained the ability to undergo alternative splicing although at a reduced level of induction. Therefore, it appears that systematic deletion of the IVS2-654 intron can yield an alternative splicing intron that has both an acceptable level of induction and a reduced size suitable for incorporation into an AAV vector. To control transgene expression, it is desirable to have an alternative splicing intron which yields a low background level of transgene expression under the conditions for exon inclusion and a high induced level of transgene expression under the conditions for exon skipping. It could be possible to obtain such a desirable intron by modifying the use of branch sites and fine tuning the strength of the alternative splice site. This is because mutating one of the branch sites significantly increased the induction level. Additionally, mutating the sequences of a splice site greatly increased the induction levels but at the same time significantly reduced the actual level of transgene expression. The size of the intron can be minimized, a series of minimal introns with modified branch sites can be produced, and/or a library can be generated to screen for a minimal intron with mutated splice sites, in order to produce an optimized intron that has low background and high induced levels of transgene expression.

For example, a minimal intron capable of efficient alternative splicing can be developed. As described herein, a deletion of a 200 by fragment from the IVS2-654 intron did not decrease the induction level. Synthesis of a small 197 by intron containing all the essential elements for splicing in the IVS2-654 intron still retained the ability to undergo alternative splicing. But the small intron had an induction level of only 2.3 fold, significantly lower than the 4.3 fold for the IVS2-654 intron To determine the maximal deletion that would still have a similar level of induction to that of the IVS2-654 intron, a plasmid containing the 200 by deletion can be further deleted, to extend the deletion toward the 5′ end, from nucleotides 150 to 33. Deletion can also be extended separately toward the 3′ end, from nucleotides 350 to 519. More deletions can also be made separately in the downstream alternative intron between nucleotides 660 and 793. For each area of deletion, the size of the fragment to be deleted can be in an increment of about 30 by initially and about 10 by later for further maximizing the size of deletion. The deletion mutants will be generated by using, for example, the Stratagene QuikChange Multi Site-Directed Mutagenesis kit. This method involves synthesis of mutant strands using primers containing desired mutations, digestion with DpnI to remove the parental plasmid, and transformation of the synthesized single-stranded plasmids into a bacterial host to be converted into double-stranded plasmids. To rapidly and quantitatively determine the induction levels of transgene expression, the luciferase assay system will be used. However, understanding the mechanism governing the action of each mutant intron would be essential to better design the intron for controlling transgene expression. Therefore, both the mRNA level and the pattern of splicing can be analyzed under a separate study. The resulting constructs will be individually transfected into 293 cells to be assayed for their induction levels of luciferase expression. After the maximal deletion for each of the three is determined, they will be combined in one construct and the resulting intron will be tested for the induction level of luciferase expression. Because use of a minimal intron would maximize the AAV cloning capacity after inserting multiple copies of the intron to control transgene expression, the minimal intron will be used to generated from this set of experiments for the rest of the proposed studies.

Generation and evaluation of modified minimal introns with mutated branch sites. As described herein, mutating one of the two potential branch sites in the upstream alternative intron increased the induction levels from 4.3 to 13 folds. To optimize the minimal intron to be used for maximizing the AAV cloning capacity after intron insertion, the four potential branch sites will be mutated separately and their induction levels of gene expression will be evaluated: The two branch sites in the upstream alternative intron are TTTTAAT at nucleotides 520-526 and CCCTAAT at 560-566, and the two branch sites in the downstream alternative intron are TGCTAAT at 813-819 and CTCTTAT at 831-837. Because the consensus branch site sequences are PyNPyUPuAPy, where Py=C or U, Pu=A or G, and the underlined A is highly conserved, the conserved A as well the upstream A will be converted to CT. Since the potential branch site CTCTTAT at 831-837 has a T instead of a conserved Pu upstream of the conserved A, only the conserved A will be mutated. The distance between the branch site and the 3′ splice site is typically eighteen bases but varies widely. To determine whether the distance has any effect on the induction level, the distance will be varied in an attempt to further optimize the induction level. The mutations will be generated by using the Stratagene QuikChange Multi Site-Directed Mutagenesis kit as described. To rapidly and quantitatively determine the induction levels of transgene expression, the luciferase assay system will be used. To understand the mechanism governing the action of each mutant intron in order to better design the intron for controlling transgene expression, both the mRNA level and the pattern of splicing will be analyzed under a separate study. The resulting constructs will be individually transfected into 293 cells to be assayed for their induction levels of luciferase expression. Optimal modifications for the upstream and the downstream alternative intron will be combined in one construct and the resulting intron will be tested for improved induction levels.

Generation and screening from a library of the minimal intron with mutated splice sites for optimized intron that has low background and high induced levels of transgene expression. To maximize the AAV cloning capacity after intron insertion, the minimal intron will be used as a template for generating a library of introns with mutated splice sites. To facilitate the screening of optimized introns, the minimal intron will be inserted into a marker expression cassette prior to the generation of the library. The marker expression cassette to be used will be one that expresses a bifunctional fusion protein between puromycin N-acetyltransferase and a truncated version of herpes simplex virus type 1 thymidine kinase (puttk). The puΔtk fusion protein has been shown to allow both positive and negative selection of cells expressing the protein using puromycin and an analog of gancyclovir, 1-(−2-deoxy-2-fluoro-1-β-D-arabino-furanosyl)-5-iodouracil (FIAU), respectively. There are several other positive/negative selectable markers that have been developed and they would serve equally well the screening of the library. The 5′ alternative splice site will be mutated to optimize the induction level of the intron. This is because the strength of the 5′ alternative splice site is significantly weaker than those of the 5′ and the 3′ splice sites as well as that of the 3′ alternative splice site according to a method of calculating the strength of a splice site. This choice is also because increasing the strength of the 5′ alternative splice site by modifying its sequences significantly increased its induction level (but at the same time decreased its overall level of transgene expression). Since in the consensus 5′ splice site sequences, ²AG↓GUPuAGU⁺⁶, where the arrow marks the exon-intron junction, the GU at positions +1 and +2 are 100% conserved, the nucleotides will be mutated at positions −2 and −1 as well as +3 to +6. To generate a library of the mutated introns, the Stratagene QuikChange Multi Site-Directed Mutagenesis kit will be used.

As an alternative method for generating a library of the mutated introns, a pair of overlapping primers, with one that spans over the 5′ alternative splice site with degenerated bases at the positions to be mutated, will be used separately in a polymerase chain reaction (PCR) with another primer either upstream or downstream of the intron. PCR products from the two separate reactions will be combined as templates for another round of PCR reaction to reconstitute the mutated introns. The resulting PCR products will be digested with restriction enzymes and used to replace the corresponding fragment in the parental plasmid, thereby creating a library of mutated introns.

The following strategy will be used to screen for an optimized intron that has low background and high induced levels of transgene expression. To enable each clone of the library to be individually expressed and selected, the library will be generated in the backbone of an Epstein-Barr Virus (EBV) plasmid. Because of its ability to be propagated as an episome, the EBV plasmid vector has been traditionally used to transform cells for drug selection. The resulting plasmid library will be transfected into 293 or HeLa cells. To select for mutated introns with high induction levels of transgene expression due to their abilities to undergo efficient exon skipping in the presence of a specific AON, the cells will be treated with the AON and selected with puromycin. Because the library would contain mutations in the 5′ alternative splice site to which the 5′ss 652/18 AON is complementary, another AON, 3′ss 579/18, will be used for the screening of the library. The 3′ss 579/18 AON is an 18-mer oligonucleotides complementary to the 3′ alternative splice site and is capable of inhibiting the inclusion of the aberrant exon with the same efficiency as that of the 5′ss 652/18 AON. To eliminate mutated introns with high background levels of transgene expression due to their inabilities to undergo efficient exon inclusion in the absence of the AON, resistant cells after the puromycin selection will be discontinued with the AON treatment. The cells will then be treated with FIAU to select for cells with low levels of puΔtk expression. The concentrations for the drug selections will be varied to allow screening of introns with the highest induction levels of transgene expression. To recover the introns from the selected cells, low molecular weight DNA will be extracted from the cells and electroporated into a bacteria host, DH5α. The recovered introns will be reinserted into the luciferase expression cassette to allow quantification of their induction levels of transgene expression. To understand the mechanism of action for each screened intron, both the mRNA level and the pattern of splicing will be analyzed under a separate study. Mutated introns with high induction levels of transgene expression thus identified will be subjected to DNA sequencing to identify their sequences.

Incorporating an Alternative Splicing Intron into an AAV Vector to Control Trausgene Expression Long-Term in an Animal Model.

Because alternative splicing can be used in vivo to control transgene expression, incorporating alternative splicing introns into an AAV vector would make it possible to control the expression of transgene over the long-term in the treated animals. Because inserting two copies of the IVS2-654 intron remarkably increased the induction level, and because the packaging limit of AAV vector is only 4.7 kb, the optimized minimal intron will be incorporated into AAV vectors to maximize the AAV cloning capacity after the intron insertion. Given that the distance between introns can influence the induction level of transgene expression (FIG. 7), AAV plasmids inserted with the optimized alternative intron at various positions and with various copy numbers will be constructed and the resulting AAV vectors will be assessed for optimal induction of transgene expression in vivo. Improving induction levels by inserting multiple copies of an intron could also be readily adapted for other gene transfer vectors that have larger packaging capacities. Thus, it is important to determine the optimal number of introns that would have a synergistic effect on the induction level of transgene expression.

Construction and evaluation in vitro of AAV plasmids carrying a marker gene inserted with the optimized alternative splicing intron. As described herein, the induction levels after inserting two introns were in reverse correlation with the distance in between the introns. The exception was that two introns in tandem only slight improved the induction level. Thus, there should be an optimal distance between introns that would yield the highest level of induction. To determine the optimal distance, two copies of the optimized intron will be inserted into the luciferase gene with various distances in between. The expected size of the resulting AAV genome would be no more than 4.0 kb, which is within the 4.7 kb AAV packaging limit (4.0 kb AAV genome=two terminal repeats+promoter+luciferase cDNA+two introns+poly A=0.29+0.56+1.65+2×0.65+0.2, the minimal intron would be no more than 650 bp). 5′ AGPu 3′ sequences, where Pu=G or A, will be chosen in the luciferase gene for insertion of the optimized intron. This criteria is based on the fact that the overwhelming majority of 5′ and 3′ splice site sequences conform to the consensus ⁻²AG↓GUPuAGU⁺⁶ and ⁻⁴NPyAG↓PuN⁺², respectively, where the arrow marks the exon-intron junction. Therefore, inserting an intron in between sequences 5′ AG and Pu 3′ would restore both the consensus 5′ and 3′ splice sites. Because the AB construct yielded the best induction level of 273 fold and had a distance of 275 by between the introns, reduction of the 275 by distance will be initiated by inserting two copies of the optimized intron, one at position B and the other at each of the candidate sites between positions A and B. This set of plasmids will have distances of 191, 118, 105, 98, 49, 30 and 15 by between the two copies of the intron. To determine whether sequences between the two copies of intron affect the induction level of transgene expression, another set of insertion plasmids will be constructed that contain one copy of the intron inserted in between nucleotides 964-965 and the other copy of the introit at each of seven candidate sites between and including nucleotides 988 and 1161. Thus, there will be distances of 197, 153, 99, 69, 52, 40 and 24 by between the two copies of the intron. Resulting constructs will be separately transfected into 293 cells to be assayed for their induction levels of transgene expression. The distance between the introns will be correlated with the induction level. To investigate whether insertion of three copies of the optimized intron will further improve the induction level of transgene expression, we will use the optimal constructs inserted with two copies of the intron selected from the above experiments for inserting another copy of the intron. The expected size of an AAV genome containing three copies of the intron would be no more than 4.65 kb, which is within the 4.7 kb AAV packaging limit (4.65 kb AAV genome=two terminal repeats+promoter+luciferase cDNA+three introns+poly A=0.29+0.56+1.65+3×0.65+0.2, the minimal intron would be no more than 650 bp). The third intron will be inserted at various positions such that there will be distances of about 800, 600, 400, 200, 100 and 50 by in between the third intron and the nearest intron. The resulting constructs will be separately transfected into 293 cells to be assayed for their induction levels of transgene expression. In the following nucleotide sequence of a fire-fly luciferase cDNA (SEQ ID NO):77), potential sites for intron insertion are underlined. Positions A-D are indicated by both the wavy underlines and the corresponding letters on the left.

| | |
|---|---|
| 1 | ATGGAAGACG CCAAAAACAT AAAGAAAGGC CCGGCGCCAT TCTATCCGCT |
| | GGAAGATGGA ACCGCTGGAG AGCAACTGCA TAAGGCTATG |
| 91 | AAGAGATACG CCCTGGTTCC TGGAACAATT GCTTTTACAG ATGCACATAT |
| | CGAGGTGGAC ATCACTTACG CTGAGTACTT CGAAATGTCC |
| 181 | GTTCGGTTGG CAGAAGCTAT GAAACGATAT GGGCTGAATA CAAATCACAG |
| | AATCGTCGTA TGCAGTGAAA ACTCTCTTCA ATTCTTTATG |
| 271 | CCGGTGTTGG GCGCGTTATT TATCGGAGTT GCAGTTGCGC CCGCGAACGA |
| | CATTTATAAT GAACGTGAAT TGCTCAACAG TATGGGCATT |
| A 361 | TCGCAGCCTA CCGTGGTGTT CGTTTCCAAA AAGGGGTTGC AAAAAATTTT |
| | GAACGTGCAA AAAAAGCTCC CAATCATCCA AAAAATTATT |
| 451 | ATCATGGATT CTAAAACGGA TTACCAGGGA TTTCAGTCGA TGTACACGTT |
| | CGTCACATCT CATCTACCTC CCGGTTTTAA TGAATACGAT |
| 541 | TTTGTGCCAG AGTCCTTCGA TAGGGACAAG ACAATTGCAC TGATCATGAA |
| | CTCCTCTGGA TCTACTGGTC TGCCTAAAGG TGTCGCTCTG |
| B 631 | CCTCATAGAA CTGCCTGCGT GAGATTCTCG CATGCCAGAG ATCCTATTTT |
| | TGGCAATCAA ATCATTCCGG ATACTGCGAT TTTAAGTGTT |
| 721 | GTTCCATTCC ATCACGGTTT TGGAATGTTT ACTACACTCG GATATTTGAT |
| | ATGTGGATTT CGAGTCGTCT TAATGTATAG ATTTGAAGAA |
| 811 | GAGCTGTTTC TGAGGAGCCT TCAGGATTAC AAGATTCAAA GTGCGCTGCT |
| | GGTGCCAACC CTATTCTCCT TCTTCGCCAA AAGCACTCTG |
| 901 | ATTGACAAAT ACGATTTATC TAATTTACAC GAAATTGCTT CTGGTGGCGC |
| | TCCCCTCTCT AAGGAAGTCG GGGAAGCGGT TGCCAAGAGG |
| 991 | TTCCATCTGC CAGGTATCAG GCAAGGATAT GGGCTCAGTG AGACTACATC |
| | AGCTATTCTG ATTACACCCG AGGGGGATGA TAAACCGGGC |
| C 1081 | GCGGTCGGTA AAGTTGTTCC ATTTTTTGAA GCGAAGGTTG TGGATCTGGA |
| | TACCGGGAAA ACGCTGGGCG TTAATCAAAG AGGCGAACTG |
| 1171 | TGTGTGAGAG GTCCTATGAT TATGTCCGGT TATGTAAACA ATCCGGAAGC |
| | GACCAACGCC TTGATTGACA AGGATGGATG GCTACATTCT |
| 1261 | GGAGACATAG CTTACTGGGA CGAAGACGAA CACTTCTTCA TCGTTGACCG |
| | CCTGAAGTCT CTGATTAAGT ACAAAGGCTA TCAGGTGGCT |
| D 1351 | CCCGCTGAAT TGGAATCCAT CTTGCTCCAA CACCCCAACA TCTTCGACGC |
| | AGGTGTCGCA GGTCTTCCCG ACGATGACGC CGGTGAACTT |
| 1441 | CCCGCCGCCG TTGTTGTTTT GGAGCACGGA AAGACGATGA CGGAAAAAGA |
| | GATCGTGGAT TACGTCGCCA GTCAAGTAAC AACCGCGAAA |
| 1531 | AAGTTGCGCG GAGGAGTTGT GTTTGTGGAC GAAGTACCGA AAGGTCTTAC |
| | CGGAAAACTC GACGCAAGAA AAATCAGAGA GATCCTCATA |
| 1621 | AAGGCCAAGA AGGGCGGAAA GATCGCCGTG TAA |

Assessment of the control of transgene expression mediated by the resulting AAV vectors in vivo over long-term. The AAV plasmids with optimal control of transgene expression to be determined as described above will be packaged into virus vectors. The vectors will be produced by utilizing an adenovirus-free production scheme which involves transfection of 293 cells with three plasmids: the recombinant AAV plasmid, an AAV-helper plasmid which supplies both the structure and the non-structure AAV genes, and an adenovirus-helper plasmid which supplies the essential helper genes for AAV vector production. The resulting AAV vector will be purified by utilizing a purification protocol which contains an iodixanol gradient and a heparin sulfate chromatography steps. The ability of the AAV vectors to mediate long-term controllable transgene expression in vivo will then be assessed by directing the purified vectors to liver by portal vein injection, as well as to skeletal muscle and heart by direct injection as described herein. The induction levels of luciferase gene expression will be determined by imaging the mice after injecting the animals with either a control or the intron specific AONs. As a control vector, AAV carrying a green fluorescent protein (GFP) expression cassette will be included in this set of experiments.

Mice will be injected with AAV vectors via different routes (e.g., portal vein, direct muscle, direct heart). Both a specific and a control AON will be administered to regulate the expression of the luciferase gene. The level of luciferase expression will be determined by whole body imaging. AAV-luc-int and AAV-GFP indicate AAV vectors carrying a luciferase expression cassette inserted with introns and a GFP expression cassette, respectively.

To determine the ability to control the expression of the luciferase gene long-term, AONs will be re-administered to the mice after the previously induced expression of luciferase returns to background levels. The newly induced expression will be monitored again by whole body imaging. This cycle of induced expression will be repeated to assess the long-term control of the transgene expression.

A potential problem with respect to inserting a third intron to yield various distances between the third and the nearest introns is that there may not be the required 5′ AGPu 3′ sequences for the insertion at the desired location. In this case, the multiple codon usage for each amino acid will be employed to create such required sequences for the insertion. For example, in the sequences of 5′ (NNX) (GPuN) 3′, where each pair of parentheses marks a codon, the nucleotide X could be converted as a silent mutation to A, thereby generating the required 5′ AGPu 3′ sequences for intron insertion. Similarly, in the sequences of 5′ (NAZ) (PuNN) 3′, nucleotide Z could be converted as a silent mutation to G. Of the twenty amino acids, eleven of them contain G and twelve of them contain A at the last position of their codons as an alternative usage. Therefore, the possibility of being able to create an insertion site at the desired location is relatively high. Repeated induction of luciferase expression in AAV infected mice would allow for the assessment of long-term control of transgene expression in vivo.

### Example 5

### Rett Syndrome Studies

There is no effective treatment for RFT. The 6- to 18-month postnatal asymptomatic window may allow interventions to be initiated before permanent neuronal damage occurs if a treatment approach is discovered. Using AAV to deliver, normal gene into CNS is a reasonable approach. An ideal vector is essential for this study. Finding a suitable vector can directly shed light on the potential for curing or ameliorating symptoms of this disease in the future. By using alternative splicing as a regulation system either over- or under-expression of the gene of interest can be avoided, expression at proper developmental periods can be controlled, and hopefully the requirements of normal function of CNS can be met. The long-term goal is to couple the ideal vector for brain specific delivery with a controlled regulation of alternative splicing in an animal model representing RTT. These studies are expected to eventually lead to the development of safe and efficacious transgene expression in patients.

Transduction patterns with different serotypes of rAAV vector in vivo. In order to determine the tropism of the different serotypes of AAV vectors in vivo, serotypes 1-5 and 8 AAV vectors were introduced into mice liver, muscle and rat retina. The transgene expression is much different in various tissues among tested serotypes. AAV1 and AAV8 can initiate the highest transgene expression in liver and muscle, however AAV5 and 4 can transduce retina cells more efficiently than other serotypes. Within 46 days post injection, transgene (green fluorescent protein, GFP) expression increased proportionally and these animals remained positive for the duration of the experiment (4 months). Using a published approach for global gene delivery, a similar analysis will be carried out in mouse brain.

The transgenes were chicken β-actin promoter (CBA) with the CMV enhancer-driven hAAT (a) and CMV immediate-early promoter-driven EGFP (b). Scores ranged from the maximum level of protein observed for each set of animals (+++++) to the lowest level of expression in the group (+).

Using complementary AON to modulate gene expression in vitro. By using a known mutant intron (human β-globin intron 2) in the transgene cassette, successful regulation of reporter gene expression after the addition of AON has been achieved.

Using intron specific GFP as reporter and the effect of correction by AON. Mutant human β-globin intron 2 was constructed into GFP cDNA and plasmid (pEGFP-mut-int) or virus (AAV2/EGFP-mut-int), which were used to transfect or infect 293 cells, respectively. The effect of AON on transgene expression was measured over time. The expression of GFP was measured 48 hr after treatment using fluorescence microscopy (Leitz D M IRB, Vashaw Scientific Inc). Efficiency of AON for correcting the aberrant splicing of the pre-mRNA is indicated by GFP positive cells.

Inserting wild or mutant intron into luciferase cDNA to modulate transgene expression. The splicing of luciferase pre-mRNA was altered by the insertion of either wild or mutant human β-globin intron 2 into the reading frame of plasmid pGL3 (Promega). Then the reconstructed plasmid (pGL3-int-luc) was transfected into 293 cells. At the same time some cells were treated with AON. The expression of luciferase was examined at 24 hr with a Microplate Luminometer (Tropix) to evaluate the splicing efficiency of the pre-mRNA. Data shows that in the presence of AON, the expression of plasmid with mutant intron increased 2-3 fold over that of the original plasmid. In addition, background can be reduced to a rather low level. The correction of the gene expression exhibits an AON dose-dependent relationship.

Using complementary AON to modulate gene expression in vivo. Since AON can modulate gene expression very efficiently in vitro, this regulation system was tested in vivo. Liver and muscle were used as target organs because tissue-specific promoters were used and expression is easy to observe using “real time” Luciferase Imaging System (Roper Scientific). The result suggests that AON can effectively correct the alternative splicing in vivo.

Identification of an ideal AAV serotype vector that specifically transduces neurons using reporter genes (e.g. green fluorescent protein, GFP). Although there are some differences in the amino acid sequences of the capsid between AAV2 and the other serotypes, the AAV2 genome or transgenes flanked by the AAV2 inverted terminal repeats can be packaged into different serotypes of capsid to form transducing virions. This provides an excellent tool to directly compare the function of serotype capsids involved in infection in vivo.

Experimental Design and Methods. The AAV2/GFP genome will be packaged into AAV serotype 1 to 8 capsid, respectively, to generate a collection of viable AAV recombinants for in vivo testing. The following experiments will be performed: 1) The same particle number of different AAV serotypes will be given to mice in order to determine which serotype can achieve the best expression in CNS. Chicken β-actin promoter (CBA) will be used to drive GFP expression in all the serotypes to be tested. This is a constitutive non tissue specific promoter. If necessary, other promoters, such as the NSE promoter, will be used in selected serotypes to further compare the intensity and specificity of the transgene expression in neurons. 2) MeCP2 cDNA will be constructed in the best AAV serotype driven by an optimal promoter and the virus will be tested for MeCP2 gene delivery in the CNS of a RTT mouse model. The gene expression will be characterized by immunohistochemistry as well as by rescue of behavior phenotype.

Identification of a suitable AAV serotype to deliver a transgene into the CNS of mice. AAV1 through 8 vectors will be prepared with the same AAV2 vector genome carrying the CBA promoter and GFP reporter gene (rAAV1-8/CBA-GFP). Virus will be made according to the 3 plasmid cotransfection method and the particle numbers will be assessed by a DNase resistant Dot blot technique. Approximately 1×10¹² particles of each serotype will be injected into the posterior cistern of each wild C57BL strain mouse brain, 15-20 min after an iv infusion of 200 μl mannitol (25%). The mice will be sacrificed at day 14 after injection. Non-injected controls will be sacrificed at the same time. Sections will be cut in the coronal or parasagittal plane and the expression of GFP in different parts of brain will be studied by using fluorescence microscope (Leitz DM IRB, Vashaw Scientific Inc), immunohistochemistry (Pierce), and Western blots, if necessary.

Testing of optimum vector(s) for MeCP2 transgene delivery into MeCP2 gene deficient animals. An MeCP2 deficient mouse model will be obtained from Jackson Laboratory. This model mimics the symptoms in human patients. By using this animal model, the effect of delivered genes in vivo can be observed. MeCP2 cDNA will be constructed into selected AAV vectors (AAV/MeCP2) and introduced into mouse brain by intracisternal injection (2×10¹⁰ particle number). Animals will be set into two groups as follows. Group 1 will be tested at 14 days after injection for gene expression, while group 2 animals will be kept alive to evaluate the survival time and observe behavioral and symptom changes longitudinally for up to 1 year.

All the animals will be monitored by the following criteria: 1) Amelioration of symptoms such as body weight, brain weight, survival time (compared with normal and mutant animal at the same age), and motor activity by using an infrared beam-activated movement-monitoring chamber (Opto-Varimax-MiniA, Columbus Instruments). Other symptoms such as tremor and heavy breathing will also be observed. Specific attention to symptoms that may result from over expression of MeCP2 will be carried out (such as failure to compete for food, size or refusal to mate). 2) Transgene expression in then brain will be detected by immunohistochemistry by using rabbit anti-MeCP2 antibody (Upstate, Lake Placid), biotinylated goat anti-rabbit IgG (Vector Laboratories), and Vectastain Elite ABC kit (Vector Laboratories).

The maximal dose of virus will be utilized with the hope of rescuing the animal model phenotype as described by Luikenhuis et al. (“Expression of MeCP2 in postmitotic neurons rescues Rett syndrome in mice” PNAS USA 101(16):6033-8 (E.pub Apr. 6, 2004); incorporated in its entirety by reference herein).

Characterizing a novel method of regulating transgene expression in mouse brain through alternative splicing. Gene deficiency can cause genetic diseases including RTT, while overexpression of certain genes can also lead to serious problems. Studies have shown that neurons can only tolerate MeCP2 expression 2-3 fold higher than normal levels before severe motor dysfunction will occur. For this reason correct levels become an issue of importance. AAV vectors are too small to carry MeCP2 tissue specific promoter cassettes. To control overexpression, an alternative splicing regulation system as described herein will be introduced into the vector cassette.

Luciferase was chosen as a reporter gene for two reasons: 1) the substrate luciferin can be injected intraperitoneally and will pass through the BBB where it can be acted on by luciferase protein expressed in this region; and 2) the Luciferase Imaging System (Roper) allows for observation of real time changes of luciferase expression in the brain without sacrificing the animal. Luciferase expression exhibited in an AON dose-dependent manner will be tested. Frequency and dose of AON to be given will be established and compared to controls (GFP vector only). Performance of this vector in the CNS will be established before testing with MeCP2 intron dependent transgene cassettes.

Studies described herein have demonstrated that AON can act by either increasing the expression of a transgene by intron correction, or by decreasing expression as the oligo is cleared. This makes transgene regulation by AON an attractive alternative to currently utilized trans-activating cassettes that have been shown to be prone to immune response. Although higher doses of AON by intravenous injection (IV) will be required to obtain the same expression level achieved by direct intracranial injection, the IV approach is much more convenient and practical.

Experimental Design and Methods. Studies described herein will be expanded by constructing either wild or mutant intron cassettes in a luciferase reporter gene. This intron dependent cassette will be constructed into selected AAV vectors driven by appropriate promoters. Virus will be produced as described above and injected directly into the posterior cistern of C57BL mice brain (2×10¹⁰ particle number/mouse). Baseline images will be collected and then AON will be given to induce the luciferase expression 2 weeks after injection. Dosage and frequency of AON administration for the rescue of transgene expression will be evaluated. The result will be observed directly by using Luciferase Imaging System (Roper) once a week.

To determine the suitable dosage of AON to be injected, different doses of AON (e.g., 0.02 μg, 1 μg, 4 μg, 20 μg and 100 μg in 100 μl saline) will be injected into the mice by intravenous injection to obtain a dosage dependent transgene expression curve. Control groups will receive the same amount of saline only. These data should help determine the dose required for AON to express intron dependent MeCP2 transgene expression in brain.

According to studies described herein, AON-induced in vivo transgene expression will decrease gradually after a certain time. So the expression of luciferase induced by the first administration of AON will theoretically decline after a certain time span. Since this decline can be observed in real time, the AON will be given again at the point when the expression drops to half of the original expression level. The transgene expression will be kept at a steady level, using Lu expression and extrapolated for similar expression time points for MeCP2. The half life of the proteins in question will determine the final conditions of this experimental approach (e.g., min vs. hr). The half-life of these proteins will be established in tissue culture using classical pulse chase experiments with S³⁵ labeled methionine. Establishment of these experimental conditions will allow for the administration of AON at a frequency that will maintain MeCP2 expression at constant level. To address issues regarding the efficiency of crossing the blood-brain bather, chemically modified AON, such as phosphorothioate oligonucleotide, can be employed. Establishment of an AAV regulated vector in brain will be of significant value to the gene therapy field as a whole and more importantly to the neurological community related to global brain disorders such as Rett Syndrome.

Use of a serotype specific vector of choice and an intron dependent splicing regulation system to deliver MeCP2 transgene into mouse brain. A regulation system dependent on mutant human β-globin intron 2 will be constructed into MeCP2 cDNA (AAV/MeCP2-mut-int). This transgene cassette will be incorporated into an ideal serotype vector and driven by a selected promoter (NSE, CBA, etc). Transgene mice will be ordered from the Jackson Laboratory. AON will be given to the mice in the amount and frequency established above. Animals will be characterized after AON delivery for transgene expression (as described above) and monitored for behavioral changes as described herein.

The foregoing examples are illustrative of the present invention, and are not to be construed as limiting thereof. The invention is described by the following claims, with equivalents of the claims to be included therein.

All publications, patent applications, patents, patent publications and other references cited herein are incorporated by reference in their entireties for the teachings relevant to the sentence and/or paragraph in which the reference is presented.

TABLE 1

| | | Inside | | | |
|---|---|---|---|---|---|
| Intron position | Pre- | A | B | C | D |
| Fold^{a} | 1.81 ± 0.19 | 4.21 ± 0.36 | 4.03 ± 0.03 | 7 ± 2.69 | 5.25 ± 1.91 |
| % of wt^{b} | 14 ± 1 | 83 ± 7 | 94 ± 4 | 123 ± 12 | 67 ± 15 |

Correction efficiency of one intron in different places relative to luciferase cDNA.
^{a}Pre-represent intron inserted between promoter and luciferase cDNA;
^{b}Fold increase of transgene expression after oligo correction compared to without oligo.
^{c}The percentage of transgene expression of plasmid with mutant intron after oligo correction relative to that with one wt intron in luciferase cDNA.

TABLE 2

| | 2 introns | | | |
|---|---|---|---|---|
| | | 2 in | 2 in | 3 introns |
| | 1 in 1 pre-^{a} | continuous | separate | 2 out 1 in |
| Folds^{b} | 76.07.9 | 10.1 ± 1.2 | 148.2 ± 28.4 | 35 |
| % of wt^{c} | 12.0 ± 0.2 | 122.7 ± 7.5 | 107.6 ± 10.7 | N/A |

Correction efficiency with insertion of multiple introns.
^{a}Pre-represent intron inserted between promoter and luciferase cDNA;
^{b}Fold increase of transgene expression after oligo correction compared without oligo.
^{c}The percentage of transgene expression of plasmid with mutant intron after oligo correction relative to that with one wt intron in luciferase cDNA.

TABLE 3

| | Shortened intron |
|---|---|
| Folds^{a} | 4.70 ± 0.92 |
| % of wt^{b} | 191.67 ± 48.58 |

Transgene correction efficiency of the shortened intron.
^{a}Fold increase of transgene expression after oligo correction compared without oligo.
^{b}The percentage of transgene expression of plasmid with mutant intron after oligo correction relative to that with one wt intron in luciferase cDNA.

What is claimed is:

1. An isolated nucleic acid comprising:
A) at least one first nucleotide sequence encoding a heterologous nucleotide sequence of interest; and
B) at least two heterologous second nucleotide sequences, wherein each heterologous second nucleotide sequence comprises:
i) a first set of splice elements defining a first intron that is removed by splicing to produce a first RNA molecule that imparts a biological function in the absence of activity at a second set of splice elements; and
ii) the second set of splice elements defining one or more introns different from said first intron, wherein said one or more introns different from said first intron are removed by splicing to produce no RNA molecule and/or a second RNA molecule that does not impart a biological function, when said second set of splice elements is active, wherein the heterologous second nucleotide sequences are selected from the group consisting of:
a) second nucleotide sequences in tandem within said first nucleotide sequence,
b) second nucleotide sequences spaced at least 25 base pairs apart within said first nucleotide sequence,
c) second nucleotide sequences spaced at least 50 base pairs apart within said first nucleotide sequence,
d) second nucleotide sequences spaced at least 75 base pairs apart within said first nucleotide sequence,
e) second nucleotide sequences spaced at least 100 base pairs apart within said first nucleotide sequence,
f) second nucleotide sequences spaced at least 200 base pairs apart within said first nucleotide sequence,
g) second nucleotide sequences spaced at least 300 base pairs apart within said first nucleotide sequence,
h) second nucleotide sequences wherein a primary second nucleotide sequence is located between a promoter and said first nucleotide sequence and a secondary second nucleotide sequence is located within said first nucleotide sequence; and
i) second nucleotide sequences wherein a primary second nucleotide sequence is located between an open reading frame and a poly(A) tail or poly A signal in said first nucleotide sequence and a secondary second nucleotide sequence located within said open reading frame of said first nucleotide sequence.

2. The nucleic acid of claim 1, wherein said first nucleotide sequence is selected from the group consisting of a nucleotide sequence encoding a protein or peptide, a nucleotide sequence having enzymatic activity as an RNA (e.g., RNAi), a nucleotide sequence encoding a ribozyme, a nucleotide sequence encoding an antisense sequence and/or a small nuclear RNA (snRNA), in any combination.

3. The nucleic acid of claim 1 or 2, comprising two or more first nucleotide sequences that can be the same or different.

4. The nucleic acid of any of claims 1-3, comprising two or more second nucleotide sequences that are the same.

5. The nucleic acid of any of claims 1-3, comprising two or more second nucleotide sequences that are different from one another.

6. A vector comprising the nucleic acid of any of claims 1-5.

7. The vector of claim 6, selected from the group consisting of a nonviral vector, a viral vector and a synthetic biological nanoparticle.

8. The vector of claim 6, selected from the group consisting of an AAV vector, an adenovirus vector, a lentivirus vector, a retrovirus vector, a herpesvirus vector, an alphavirus vector, a poxvirus vector a baculovirus vector and a chimeric virus vector.

9. A cell comprising the nucleic acid of any of claims 1-5.

10. A cell comprising the vector of any of claims 6-8.

11. A composition comprising the nucleic acid of any of claims 1-5 and a pharmaceutically acceptable carrier.

12. A composition comprising the vector of any of claims 6-8 and a pharmaceutically acceptable carrier.

13. A composition comprising the cell of any of claims 9-10 and a pharmaceutically acceptable carrier.

14. A method for producing a protein, comprising;
a) contacting a blocking oligonucleotide with the nucleic acid of any of claims 1-5 under conditions that permit splicing, wherein the blocking oligonucleotide blocks a member of the second set of splice elements, resulting in removal of the first intron by splicing and production of the first RNA; and
b) translating the first RNA to produce the protein.

15. A method for producing an RNA that imparts a biological function, comprising;
a) contacting a blocking oligonucleotide with the nucleic acid of any of claims 1-5 under conditions that permit splicing, wherein the blocking oligonucleotide blocks a member of the second set of splice elements, resulting in removal of the first intron by splicing and production of the first RNA; and
b) translating the first RNA to produce the RNA that imparts biological function.

16. The method of any of claims 14-15, wherein the blocking oligonucleotide is introduced into a cell comprising the nucleic acid.

17. The method of claim 16, wherein the cell is in an animal.

18. The method of claim 17, wherein the animal is a human.

19. The method of any of claims 14-18, wherein the blocking oligonucleotide does not activate RNase H.

20. The method of any of claims 14-19, wherein the blocking oligonucleotide comprises a modified internucleotide bridging phosphate residue selected from the group consisting of methyl phosphorothioates, phosphoromorpholidates, phosphoropiperazidates and phosphoramidates.

21. The method of any of claims 14-20, wherein the blocking oligonucleotide comprises a nucleotide having a loweralkyl substituent at the 2′ position thereof.

22. The method of any of claims 14-21, wherein the blocking oligonucleotide is from eight to 50 nucleotides in length.

23. A method for producing a protein, comprising:
a) contacting a small molecule with the nucleic acid of any of claims 1-5 under conditions which permit splicing, wherein the small molecule blocks a member of the second set of splice elements, resulting in removal of the first intron and production of the first RNA; and
b) translating the first RNA to produce the protein.

24. A method for producing an RNA that imparts a biological function, comprising:
a) contacting a small molecule with the nucleic acid of any of claims 1-5 under conditions which permit splicing, wherein the small molecule blocks a member of the second set of splice elements, resulting in removal of the first intron and production of the first RNA; and
b) translating the first RNA to produce the RNA that imparts a biological function.

25. The method of any of claims 23-24, wherein the small molecule is introduced into a cell comprising the nucleic acid.

26. The method of claim 25, wherein the cell is in an animal.

27. The method of claim 26, wherein the animal is a human.

28. A method of regulating production of a heterologous RNA that imparts a biological function in a subject, comprising:
a) introducing into the subject the nucleic acid of any of claims 1-5; and
b) introducing into the subject a blocking oligonucleotide and/or small molecule that blocks a member of the second set of splice elements, at a time when production of the heterologous RNA is desired, thereby regulating production of the RNA in the subject.

29. A method of regulating production of a heterologous protein in a subject, comprising:
a) introducing into the subject the nucleic acid of any of claims 1-5; and
b) introducing into the subject a blocking oligonucleotide and/or small molecule that blocks a member of the second set of splice elements, at a time when production of the heterologous protein is desired, thereby regulating production of the protein in the subject.

30. A method of treating a disorder in a subject, comprising:
a) introducing into the subject the nucleic acid of any of claims 1-5; and
b) introducing into the subject a blocking oligonucleotide and/or small molecule, thereby treating the disorder in the subject.

31. A method of identifying a compound that blocks a member of the second set of splice elements of the nucleic acid of claim 1, comprising:
a) contacting the nucleic acid of claim 1 with the compound under conditions that permit splicing; and
b) detecting the production of the first RNA of claim 1 and/or the production of the second RNA, whereby the production of the first RNA of claim 1 identifies a compound that blocks a member of the second set of splice elements of claim 1.

32. A method for inhibiting production of a heterologous RNA that imparts a biological function, comprising:
a) contacting a small molecule with the nucleic acid of any of claims 1-5 under conditions which permit splicing, wherein the small molecule blocks a member of the first set of splice elements, resulting in removal of the second intron, thereby inhibiting production of the first RNA.

33. A method for inhibiting production of a heterologous protein, comprising:
a) contacting a small molecule with the nucleic acid of any of claims 1-5 under conditions which permit splicing, wherein the small molecule blocks a member of the first set of splice elements, resulting in removal of the second intron, thereby inhibiting production of the first RNA.

34. A method for inhibiting production of a heterologous RNA that imparts a biological function, comprising:
a) contacting a blocking oligonucleotide with the nucleic acid of any of claims 1-5 under conditions which permit splicing, wherein the blocking oligonucleotide blocks a member of the first set of splice elements, resulting in removal of the second intron, thereby inhibiting production of the first RNA.

35. A method of inhibiting production of a heterologous protein, comprising:
a) contacting a blocking oligonucleotide with the nucleic acid of any of claims 1-5 under conditions which permit splicing, wherein the blocking oligonucleotide blocks a member of the first set of splice elements, resulting in removal of the second intron, thereby inhibiting production of the first RNA.
